(19) United States
(12) Patent Application Publication
(10) Pub. No.: US 2012/0245171 A1
(43) Pub. Date: Sep. 27, 2012

(54) BENZPYRAZOLE DERIVATIVES AS INHIBITORS OF PI3 KINASES

(75) Inventors: Ian Robert Baldwin, Stevenage (GB); Kenneth David Down, Stevenage (GB); Paul Faulder, Stevenage (GB); Simon Gaines, Stevenage (GB); Julie Nicole Hamblin, Stevenage (GB); Katherine Louise Jones, Stevenage (GB); Paul Spencer Jones, Stevenage (GB); Joelle Le, Stevenage (GB); Christopher James Lunniss, Stevenage (GB); Nigel James Parr, Stevenage (GB); Timothy John Ritchie, Stevenage (GB); John Edward Robinson, Stevenage (GB); Juliet Kay Simpson, Stevenage (GB); Christian Alan Paul Smethurst, Harlow (GB)

(73) Assignee: GLAXO GROUP LIMITED, Greenford Middlesex (GB)

(21) Appl. No.: 13/513,063

(22) PCT Filed: Dec. 3, 2010
(86) PCT No.: PCT/EP10/68797

§ 371 Date: May 31, 2012

Related U.S. Application Data
(60) Provisional application No. 61/266,346, filed on Dec. 3, 2009.

(51) Int. Cl. A61K 31/4985; C07D 417/14; A61K 31/496; C07D 413/14; A61K 31/5377; C07D 471/04; A61P 37/00; A61P 29/00; A61P 9/00; A61P 35/00; A61P 25/28; A61P 1/18; A61P 13/12; A61P 7/04; A61P 15/08; A61P 37/06; A61P 11/00; A61P 25/00; A61P 11/06; A61P 37/08; A61P 17/00; A61P 1/00; A61P 7/02; A61P 9/10; C07D 487/04

(52) U.S. Cl. 514/234.2; 544/349; 514/249; 544/369; 514/254.02; 544/364; 514/253.1; 544/140; 514/234.5; 544/131; 544/127

(57) ABSTRACT

The invention is directed to certain novel compounds. Specifically, the invention is directed to compounds of formula (I):

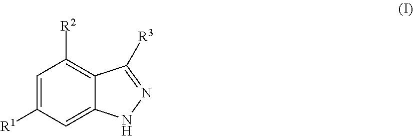

and salts thereof. The compounds of the invention are inhibitors of PI3-kinase activity.

### FIELD OF THE INVENTION

The present invention is directed to certain novel compounds which are inhibitors of the activity or function of the phosphoinositide 3′OH kinase family (hereinafter PI3-kinases), processes for their preparation, pharmaceutical compositions comprising the compounds, and the use of the compounds or the compositions in the treatment of various disorders. More specifically, the compounds of the invention are inhibitors of the activity or function of, for example, PI3Kδ, PI3Kα, PI3Kβ and/or PI3Kγ. Compounds which are inhibitors of the activity or function of PI3-kinases may be useful in the treatment of disorders such as respiratory diseases including asthma and chronic obstructive pulmonary disease (COPD); allergic diseases including allergic rhinitis and atopic dermatitis; autoimmune diseases including rheumatoid arthritis and multiple sclerosis; inflammatory disorders including inflammatory bowel disease; cardiovascular diseases including thrombosis and atherosclerosis; hematologic malignancies; cystic fibrosis; neurodegenerative diseases; pancreatitis; multiorgan failure; kidney diseases; platelet aggregation; cancer; sperm motility; transplantation rejection; graft rejection; lung injuries; and pain including pain associated with rheumatoid arthritis or osteoarthritis, back pain, general inflammatory pain, post hepatic neuralgia, diabetic neuropathy, inflammatory neuropathic pain (trama), trigeminal neuralgia and central pain.

### BACKGROUND OF THE INVENTION

Cellular membranes represent a large store of second messengers that can be enlisted in a variety of signal transduction pathways. In relation to function and regulation of effector enzymes in phospholipids signaling pathways, class I PI3-kinases (e.g. PI3Kdelta) generate second messengers from the membrane phospholipid pools. Class I PI3Ks convert the membrane phospholipid PI(4,5)P₂ into PI(3,4,5)P₃, which functions as a second messenger. PI and PI(4)P are also substrates of PI3K and can be phosphorylated and converted into PI3P and PI(3,4)P₂, respectively. In addition, these phosphoinositides can be converted into other phosphoinositides by 5′-specific and 3′-specific phophatases. Thus, PI3K enzymatic activity results either directly or indirectly in the generation of two 3′-phosphoinositide subtypes which function as second messengers in intracellular signal transduction pathways (Trends Biochem. Sci. 22(7) p. 267-72 (1997) by Vanhaesebroeck et al.; Chem. Rev. 101(8) p. 2365-80 (2001) by Leslie et al.; Annu. Rev. Cell Dev. Biol. 17 p. 615-75 (2001) by Katso et al.; and Cell. Mol. Life Sci. 59(5) p. 761-79 (2002) by Toker). To date, eight mammalian PI3Ks have been identified, divided into three main classes (I, II, and III) on the basis of sequence homology, structure, binding partners, mode of activation, and substrate preference. In vitro, class I PI3Ks can phosphorylate phosphatidylinositol (PI), phosphatidylinositol-4-phosphate (PI4P), and phosphatidylinositol-4,5-bisphosphate (PI(4,5)P₂) to produce phosphatidylinositol-3-phosphate (PI3P), phosphatidylinositol-3,4-bisphosphate (PI(3,4)P₂, and phosphatidylinositol-3,4,5-trisphosphate (PI(3,4,5)P₃, respectively. Class II PI3Ks can phosphorylate PI and PI4P. Class III PI3Ks can only phosphorylate PI (Vanhaesebroeck et al. (1997), above; Vanhaesebroeck et al., Exp. Cell Res. 253(1) p. 239-54 (1999); and Leslie et al. (2001), above).

Class I PI3K is a heterodimer consisting of a p110 catalytic subunit and a regulatory subunit, and the family is further divided into class Ia and class Ib enzymes on the basis of regulatory partners and mechanism of regulation. Class Ia enzymes consist of three distinct catalytic subunits (p110α, p110β, and p110δ) that dimerise with five distinct regulatory subunits (p85α, p55α, p50α, p85β, and p55γ), with all catalytic subunits being able to interact with all regulatory subunits to form a variety of heterodimers. Class Ia PI3K are generally activated in response to growth factor-stimulation of receptor tyrosine kinases, via interaction of the regulatory subunit SH2 domains with specific phospho-tyrosine residues of the activated receptor or adaptor proteins such as IRS-1. Small GTPases (ras as an example) are also involved in the activation of PI3K in conjunction with receptor tyrosine kinase activation. Both p110α and p110β are constitutively expressed in all cell types, whereas p110δ expression is more restricted to leukocyte populations and some epithelial cells. In contrast, the single Class Ib enzyme consists of a p110γ catalytic subunit that interacts with a p101 regulatory subunit. Furthermore, the Class Ib enzyme is activated in response to G-protein coupled receptor (GPCR) systems and its expression appears to be limited to leukocytes.

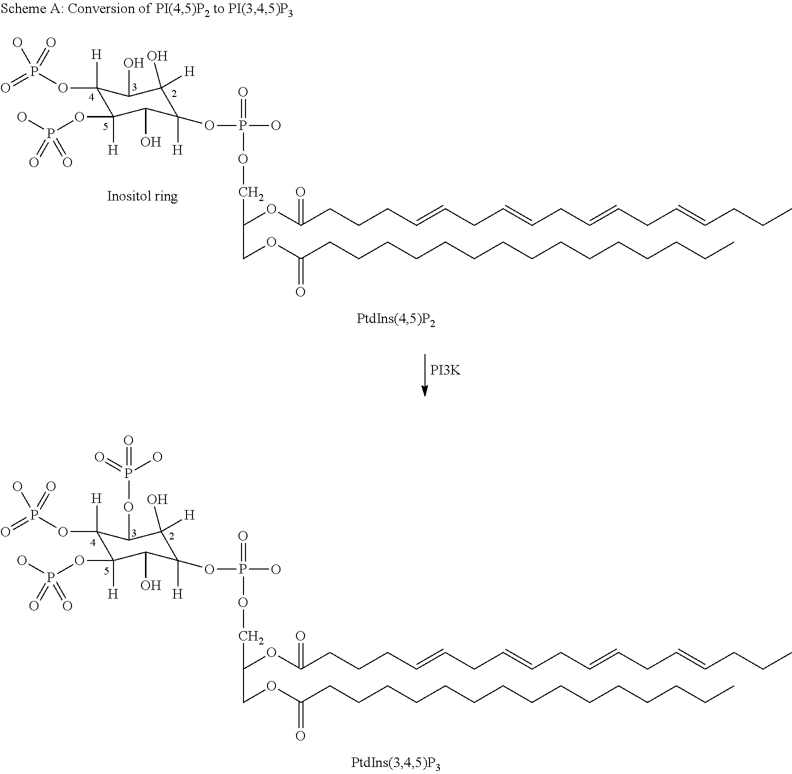

As illustrated in Scheme A above, phosphoinositide 3-kinases (PI3Ks) phosphorylate the hydroxyl of the third carbon of the inositol ring. The phosphorylation of phosphoinositides to generate PtdIns(3,4,5)P₃, PtdIns(3,4)P₂ and PtdIns(3)P, produces second messengers for a variety of signal transduction pathways, including those essential to cell proliferation, cell differentiation, cell growth, cell size, cell survival, apoptosis, adhesion, cell motility, cell migration, chemotaxis, invasion, cytoskeletal rearrangement, cell shape changes, vesicle trafficking and metabolic pathway (Katso et al. (2001), above; and Mol. Med. Today 6(9) p. 347-57 (2000) by Stein et al.).

The activity of P13-kinases responsible for generating these phosphorylated signalling products was originally identified as being associated with viral oncoproteins and growth factor receptor tyrosine kinases that phosphorylate phosphatidylinositol (PI) and its phosphorylated derivatives at the 3′-hydroxyl of the inositol ring (Panayotou et al. Trends Cell Biol. 2 p. 358-60 (1992)). However, more recent biochemical studies have revealed that class I PI3-kinases (e.g. class IA isoform PI3Kδ) are dual-specific kinase enzymes, meaning they display both lipid kinase (phosphorylation of phosphoinositides) as well as protein kinase activity, and are capable of phosphorylation of other protein as substrates, including auto-phosphorylation as an intramolecular regulatory mechanism (EMBO J. 18(5) p. 1292-302 (1999) by Vanhaesebroeck et al.). Cellular processes in which PI3Ks play an essential role include suppression of apoptosis, reorganization of the actin skeleton, cardiac myocyte growth, glycogen synthase stimulation by insulin, TNFα-mediated neutrophil priming and superoxide generation, and leukocyte migration and adhesion to endothelial cells.

PI3-kinase activation is believed to be involved in a wide range of cellular responses including cell growth, differentiation, and apoptosis (Parker, Current Biology, 5(6) p. 577-79 (1995); and Yao et al. Science 267(5206) p. 2003-06 (1995)). PI3-kinase appears to be involved in a number of aspects of leukocyte activation. A p85-associated PI3-kinase has been shown to physically associate with the cytoplasmic domain of CD28, which is an important costimulatory molecule for the activation of T-cells in response to antigen (Pagès et al. Nature 369 p. 327-29 (1994); and Rudd, Immunity 4 p. 527-34 (1996)). Activation of T cells through CD28 lowers the threshold for activation by antigen and increases the magnitude and duration of the proliferative response. These effects are linked to increases in the transcription of a number of genes including interleukin-2 (IL2), an important T cell growth factor (Fraser et al. Science 251(4991) p. 313-16 (1991)).

PI3Kγ has been identified as a mediator of G beta-gamma-dependent regulation of JNK activity, and G beta-gamma are subunits of heterotrimeric G proteins (Lopez-llasaca et al. J. Biol. Chem. 273(5) p. 2505-8 (1998)). Recently, (Laffargue et al. Immunity 16(3) p. 441-51 (2002)) it has been described that PI3Kγ relays inflammatory signals through various G(i)-coupled receptors and is central to mast cell function, stimuli in the context of leukocytes, and immunology including cytokines, chemokines, adenosines, antibodies, integrins, aggregation factors, growth factors, viruses or hormones for example (J. Cell Sci. 114 (Pt 16) p. 2903-10 (2001) by Lawlor et al.; Laffargue et al. (2002), above; and Curr. Opinion Cell Biol. 14(2) p. 203-13 (2002) by Stephens et al.).

Specific inhibitors against individual members of a family of enzymes provide invaluable tools for deciphering functions of each enzyme. Two compounds, LY294002 and wortmannin (hereinafter), have been widely used as PI3-kinase inhibitors. These compounds are non-specific PI3K inhibitors, as they do not distinguish among the four members of Class I PI3-kinases. For example, the IC₅₀ values of wortmannin against each of the various Class I PI3-kinases are in the range of 1-10 nM. Similarly, the IC₅₀ values for LY294002 against each of these PI3-kinases is about 15-20 μM (Fruman et al. Ann. Rev. Biochem. 67 p. 481-507 (1998)), also 5-10 microM on CK2 protein kinase and some inhibitory activity on phospholipases. Wortmannin is a fungal metabolite which irreversibly inhibits PI3K activity by binding covalently to the catalytic domain of this enzyme. Inhibition of PI3K activity by wortmannin eliminates subsequent cellular response to the extracellular factor. For example, neutrophils respond to the chemokine fMet-Leu-Phe (fMLP) by stimulating PI3K and synthesizing PtdIns (3, 4, 5)P₃. This synthesis correlates with activation of the respiratory burst involved in neutrophil destruction of invading microorganisms. Treatment of neutrophils with wortmannin prevents the fMLP-induced respiratory burst response (Thelen et al. Proc. Natl. Acad. Sci. USA 91 p. 4960-64 (1994)). Indeed, these experiments with wortmannin, as well as other experimental evidence, show that PI3K activity in cells of hematopoietic lineage, particularly neutrophils, monocytes, and other types of leukocytes, is involved in many of the non-memory immune response associated with acute and chronic inflammation.

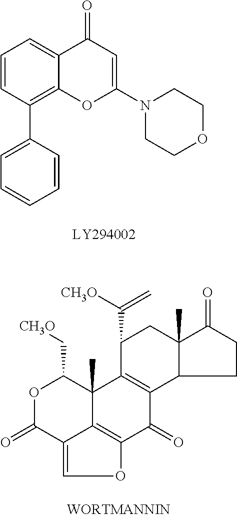

Based on studies using wortmannin, there is evidence that PI3-kinase function is also required for some aspects of leukocyte signaling through G-protein coupled receptors (Thelen et al. (1994), above). Moreover, it has been shown that wortmannin and LY294002 block neutrophil migration and superoxide release.

It is now well understood that deregulation of oncogenes and tumour suppressor genes contributes to the formation of malignant tumours, for example by way of increased cell growth and proliferation or increased cell survival. It is also now known that signaling pathways mediated by the PI3K family have a central role in a number of cell processes including proliferation and survival, and deregulation of these pathways is a causative factor a wide spectrum of human cancers and other diseases (Katso et al. Annual Rev. Cell Dev. Biol. (2001) 17 p. 615-675 and Foster et al. J. Cell Science (2003) 116(15) p. 3037-3040). PI3K effector proteins initiate signalling pathways and networks by translocating to the plasma membrane through a conserved Pleckstrin Homology (PH) domain, which specifically interacts with PtdIns(3,4,5)P3 (Vanhaesebroeck et al. Annu. Rev. Biochem. (2001) 70 p. 535-602). The effector proteins signalling through PtdIns(3,4,5)P3 and PH domains include Serine/Threonine (Ser/Thr) kinases, Tyrosine kinases, Rac or Arf GEFs (Guanine nucleotide exchange factors) and Arf GAPs (GTPase activating proteins).

In B and T cells PI3Ks have an important role through activation of the Tec family of protein tyrosine kinases which include Bruton's tyrosine kinase (BTK) in B cells and Interleukin-2-inducible T-cell kinase (ITK) in T cells. Upon PI3K activation, BTK or ITK translocate to the plasma membrane where they are subsequently phosphorylated by Src kinases. One of the major targets of activated ITK is phospholipase C-gamma (PLCγ1), which hydrolyses PtdIns(4,5)P2 into Ins(3,4,5)P3 and initiates an intracellular increase in calcium levels and diacylglycerol (DAG) which can activate Protein Kinases C in activated T cells.

Unlike the Class IA p110α and p110β, p110δ is expressed in a tissue restricted fashion. Its high expression level in lymphocytes and lymphoid tissues suggests a role in PI3K-mediated signalling in the immune system. The p110δ kinase dead knock-in mice are also viable and their phenotype is restricted to defects in immune signalling (Okkenhaug et al. Science (2002) 297 p. 1031-4). These transgenic mice have offered insight into the function of PI3Kδ in B-cell and T-cell signalling. In particular, p110δ is required for PtdIns(3,4,5)P3 formation downstream of CD28 and/or T cell Receptor (TCR) signalling. A key effect of PI3K signalling downstream of TCR is the activation of Akt, which phosphorylates anti-apoptotic factors as well as various transcription factors for cytokine production. As a consequence, T cells with inactive p110δ have defects in proliferation and Th1 and Th2 cytokine secretion. Activation of T cells through CD28 lowers the threshold for TCR activation by antigen and increases the magnitude and duration of the proliferative response. These effects are mediated by the PI3Kδ-dependent increase in the transcription of a number of genes including IL2, an important T cell growth factor.

Therefore, PI3K inhibitors are anticipated to provide therapeutic benefit via its role in modulating T-cell mediated inflammatory responses associated to respiratory diseases such as asthma, COPD and cystic fibrosis. In addition, there is indication that T-cell directed therapies may provide corticosteroid sparing properties (Alexander et al. Lancet (1992) 339 p. 324-8) suggesting that it may provide a useful therapy either as a standalone or in combination with inhaled or oral glucocorticosteroids in respiratory diseases. A PI3K inhibitor might also be used alongside other conventional therapies such as a long acting beta-agonist (LABA) in asthma.

In the vasculature, PI3Kδ is expressed by endothelial cells and participates in neutrophil trafficking by modulating the proadhesive state of these cells in response to TNFalpha (Puri et al. Blood (2004) 103(9) p. 3448-56). A role for PI3Kδ in TNFalpha-induced signalling of endothelial cells is demonstrated by the pharmacological inhibition of Akt phosphorylation and PDK1 activity. In addition, PI3Kδ is implicated in vascular permeability and airway tissue edema through the VEGF pathway (Lee et al. J. Allergy Clin. Immunol. (2006) 118(2) p. 403-9). These observations suggest additional benefits of PI3Kδ inhibition in asthma by the combined reduction of leukocyte extravasation and vascular permeability associated with asthma. In addition, PI3Kδ activity is required for mast cell function both in vitro and in vivo (Ali et al. Nature (2004) 431 p. 1007-11; and Ali et al. J. Immunol. (2008) 180(4) p. 2538-44) further suggesting that PI3K inhibition should be of therapeutic benefit for allergic indications such asthma, allergic rhinitis and atopic dermatitis.

The role of PI3Kδ in B cell proliferation, antibody secretion, B-cell antigen and IL-4 receptor signalling, B-cell antigen presenting function is also well established Okkenhaug et al. (2002), above; Al-Alwan et al. J. Immunol. (2007) 178(4) p. 2328-35; and Bilancio et al. Blood (2006) 107(2) p. 642-50) and indicates a role in autoimmune diseases such as rheumatoid arthritis or systemic lupus erythematosus. Therefore PI3K inhibitors may also be of benefit for these indications.

Pharmacological inhibition of PI3Kδ inhibits fMLP-dependent neutrophil chemotaxis on an ICAM coated agarose matrix integrin-dependent biased system (Sadhu et al. J. Immunol. (2003) 170(5) p. 2647-54). Inhibition of PI3Kδ regulates neutrophil activation, adhesion and migration without affecting neutrophil mediated phagocytosis and bactericidal activity over Staphylococcus aureus (Sadhu et al. Biochem. Biophys. Res. Commun. (2003) 308(4) p. 764-9). Overall, the data suggest that PI3Kδ inhibition should not globally inhibit neutrophil functions required for innate immune defence. PI3Kδ's role in neutrophils offers further scope for treating inflammatory diseases involving tissue remodeling such as COPD or rheumatoid arthritis.

In addition, there is also good evidence that class Ia PI3K enzymes also contribute to tumourigenesis in a wide variety of human cancers, either directly or indirectly (Vivanco and Sawyers, Nature Reviews Cancer (2002) 2(7) p. 489-501). For example, inhibition of PI3Kδ may have a therapeutic role for the treatment of malignant haematological disorders such as acute myeloid leukaemia (Billottet et al. Oncogene (2006) 25(50) p. 6648-59). Moreover, activating mutations within p110α (PIK3CA gene) have been associated with various other tumors such as those of the colon and of the breast and lung (Samuels et al. Science (2004) 304(5670) p. 554).

It has also been shown that PI3K is involved in the establishment of central sensitization in painful inflammatory conditions (Pezet et al. The J. of Neuroscience (2008) 28 (16) p. 4261-4270).

Attempts have been made to prepare compounds which inhibit PI3-kinase activity and a number of such compounds have been disclosed in the art. However, in view of the number of pathological responses which are mediated by PI3-kinases, there remains a continuing need for inhibitors of P13-kinase which can be used in the treatment of a variety of conditions.

The present inventors have discovered novel compounds which are inhibitors of PI3-kinase activity. Compounds which are PI3-kinase inhibitors may be useful in the treatment of disorders associated with inappropriate PI3-kinase activity, for example in the treatment and prevention of disorders mediated by PI3-kinase mechanisms. Such disorders include respiratory diseases including asthma and chronic obstructive pulmonary disease (COPD); allergic diseases including allergic rhinitis and atopic dermatitis; autoimmune diseases including rheumatoid arthritis and multiple sclerosis; inflammatory disorders including inflammatory bowel disease; cardiovascular diseases including thrombosis and atherosclerosis; hematologic malignancies; cystic fibrosis; neurodegenerative diseases; pancreatitis; multiorgan failure; kidney diseases; platelet aggregation; cancer; sperm motility; transplantation rejection; graft rejection; lung injuries; and pain including pain associated with rheumatoid arthritis or osteoarthritis, back pain, general inflammatory pain, post hepatic neuralgia, diabetic neuropathy, inflammatory neuropathic pain (trama), trigeminal neuralgia and central pain.

In one embodiment, compounds of the invention may show selectivity for PI3-kinases over other kinases.

In one embodiment, compounds of the invention may show selectivity for PI3Kδ over other PI3-kinases.

### SUMMARY OF THE INVENTION

The invention is directed to certain novel compounds. Specifically, the invention is directed to compounds of formula (I)

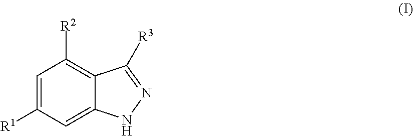

wherein R¹, R² and R³ are as defined below, and salts thereof.

The compounds are inhibitors of PI3-kinase activity. Compounds which are PI3-kinase inhibitors may be useful in the treatment of disorders associated with inappropriate PI3-kinase activity, such as asthma and chronic obstructive pulmonary disease (COPD). Accordingly, the invention is further directed to pharmaceutical compositions comprising a compound of formula (I) or a pharmaceutically acceptable salt thereof. The invention is still further directed to methods of inhibiting PI3-kinase activity and treatment of disorders associated therewith using a compound of formula (I) or a pharmaceutically acceptable salt thereof, or a pharmaceutical composition comprising a compound of formula (I) or a pharmaceutically acceptable salt thereof. The invention is yet further directed towards processes for the preparation for the compounds of the invention.

### DETAILED DESCRIPTION OF THE INVENTION

In one embodiment, the invention is directed to compounds of formula (I)

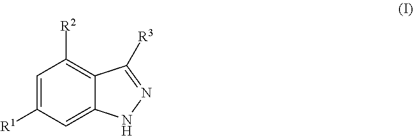

wherein

R¹ is 9- or 10-membered bicyclic heteroaryl wherein the 9- or 10-membered bicyclic heteroaryl contains from one to three heteroatoms independently selected from oxygen and nitrogen and is optionally substituted by C₁₋₆alkyl, C₃₋₆cycloalkyl, halo or —CN; or pyridinyl optionally substituted by one or two substituents independently selected from C₁₋₆alkyl, —OR⁴, halo, and —NHSO₂R⁵;

R² is —NHCOR⁶,

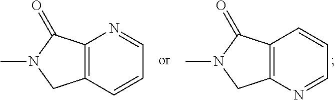

R³ is hydrogen or fluoro;

R⁴ and R⁷ are each independently hydrogen or C₁₋₆alkyl;

R⁵ is C₁₋₆alkyl or phenyl optionally substituted by one or two substituents independently selected from C₁₋₆alkyl, halo, —CF₃ and —OR⁷;

R⁶ is 5-membered heteroaryl wherein the 5-membered heteroaryl contains from one to three heteroatoms independently selected from oxygen, nitrogen and sulphur and is substituted by —CH₂NR⁸R⁹, —CH₂tetrahydropyran or —CH₂phenyl wherein the phenyl is substituted by halo; —(CH₂)ₙNR¹⁰R¹¹; or —CH₂tetrahydropyran;

R⁸ and R⁹, together with the nitrogen atom to which they are attached, are linked to form a 6-membered heterocyclyl or a 9-membered bicyclic heterocyclyl wherein the 6-membered heterocyclyl and the 9-membered bicyclic heterocyclyl each optionally containing a further nitrogen atom and the 6-membered heterocyclyl is substituted by three C₁₋₆alkyl substituents;

R¹⁰ and R¹¹, together with the nitrogen atom to which they are attached, are linked to form a 6-membered heterocyclyl optionally containing an oxygen atom; and

n is 1, 2 or 3;

and salts thereof (hereinafter “compounds of the invention”).

In one embodiment, R¹ is 9-membered bicyclic heteroaryl wherein the 9-membered bicyclic heteroaryl contains from one to three heteroatoms independently selected from oxygen and nitrogen and is optionally substituted by C₁₋₆alkyl, halo or —CN; or pyridinyl optionally substituted by one or two substituents independently selected from C₁₋₆alkyl, —OR⁴, halo, and —NHSO₂R⁵. In another embodiment, R¹ is 9-membered bicyclic heteroaryl wherein the 9-membered bicyclic heteroaryl contains from one to three nitrogen atoms and is optionally substituted by halo. In another embodiment, R¹ is indolyl. In a further embodiment, R¹ is pyridinyl optionally substituted by one or two substituents independently selected from —OR⁴, halo, and —NHSO₂R⁵.

In one embodiment, R² is —NHCOR⁶.

In one embodiment, R³ is hydrogen.

In one embodiment, R⁴ is methyl.

In one embodiment, R⁵ is methyl or phenyl. In a further embodiment, R⁵ is methyl.

In one embodiment, R⁶ is 5-membered heteroaryl wherein the 5-membered heteroaryl contains from one or two heteroatoms independently selected from nitrogen and sulphur and is substituted by —CH₂NR⁸R⁹ or —CH₂tetrahydropyran; or —(CH₂)ₙNR¹⁰R¹¹. In a further embodiment, R⁶ is 5-membered heteroaryl wherein the 5-membered heteroaryl contains from one or two heteroatoms independently selected from nitrogen and sulphur and is substituted by —CH₂tetrahydropyran.

In one embodiment, R⁷ is methyl.

In one embodiment, R⁸ and R⁹, together with the nitrogen atom to which they are attached, are linked to form a 6-membered heterocyclyl or a 9-membered bicyclic heterocyclyl wherein the 6-membered heterocyclyl and the 9-membered bicyclic heterocyclyl each contain a further nitrogen atom and the 6-membered heterocyclyl is substituted by three C₁₋₆alkyl substituents. In a further embodiment, R⁸ and R⁹, together with the nitrogen atom to which they are attached, are linked to form a 6-membered heterocyclyl wherein the 6-membered heterocyclyl contains a further nitrogen atom and is substituted by three C₁₋₆alkyl substituents.

In one embodiment, R¹⁰ and R¹¹, together with the nitrogen atom to which they are attached, are linked to form a morpholinyl.

In one embodiment, n is 1. In a further embodiment, n is 3.

It is to be understood that the present invention covers all combinations of substituent groups described hereinabove.

Compounds of the invention include the compounds of Examples 1 to 22 and salts thereof.

In one embodiment, the compound of the invention is:

2-[(8aS)-hexahydropyrrolo[1,2-a]pyrazin-2(1H)-ylmethyl]-N-[6-(1H-indol-4-yl)-1H-indazol-4-yl]-1,3-thiazole-4-carboxamide;

2-{[(3R,5S)-3,5-dimethyl-4-(1-methylethyl)-1-piperazinyl]methyl}-N-[6-(1H-indol-4-yl)-1H-indazol-4-yl]-1,3-thiazole-4-carboxamide;

2-{[(3R,5S)-3,5-dimethyl-4-(1-methylethyl)-1-piperazinyl]methyl}-N-(6-{6-(methyloxy)-5-[(methylsulfonyl)amino]-3-pyridinyl}-1H-indazol-4-yl)-1,3-thiazole-4-carboxamide;

N-(6-{6-(methyloxy)-5-[(methylsulfonyl)amino]-3-pyridinyl}-1H-indazol-4-yl)-1-(tetrahydro-2H-pyran-4-ylmethyl)-1H-pyrazole-3-carboxamide;

N-[6-(1H-indol-4-yl)-1H-indazol-4-yl]-1-(tetrahydro-2H-pyran-4-ylmethyl)-1H-pyrazole-3-carboxamide;

N-[6-(1H-indol-4-yl)-1H-indazol-4-yl]-1-(tetrahydro-2H-pyran-4-ylmethyl)-1H-pyrazole-5-carboxamide;

1-[(2-chlorophenyl)methyl]-N-[6-(1H-indol-4-yl)-1H-indazol-4-yl]-1H-pyrazole-3-carboxamide;

1-[(4-chlorophenyl)methyl]-N-[6-(1H-indol-4-yl)-1H-indazol-4-yl]-1H-pyrazole-3-carboxamide;

6-[6-(1H-indol-4-yl)-1H-indazol-4-yl]-6,7-dihydro-5H-pyrrolo[3,4-b]pyridin-5-one;

6-[6-(1H-indol-4-yl)-1H-indazol-4-yl]-5,6-dihydro-7H-pyrrolo[3,4-b]pyridin-7-one;

N-[6-(1H-indol-4-yl)-1H-indazol-4-yl]-2-(tetrahydro-2H-pyran-4-yl)acetamide;

N-[6-(1H-indol-4-yl)-1H-indazol-4-yl]-2-(1-piperidinyl)acetamide;

N-[6-(1H-indol-4-yl)-1H-indazol-4-yl]-4-(4-morpholinyl)butanamide;

N-[6-(1H-indol-4-yl)-1H-indazol-4-yl]-2-(4-morpholinyl)acetamide;

2-(4-morpholinyl)-N-(6-{5-[(phenylsulfonyl)amino]-3-pyridinyl}-1H-indazol-4-yl)acetamide;

N-(6-{6-chloro-5-[(methylsulfonyl)amino]-3-pyridinyl}-1H-indazol-4-yl)-2-(4-morpholinyl)acetamide;

N-(6-{6-methyl-5-[(methylsulfonyl)amino]-3-pyridinyl}-1H-indazol-4-yl)-2-(4-morpholinyl)acetamide;

N-(6-{5-[(methylsulfonyl)amino]-3-pyridinyl}-1H-indazol-4-yl)-2-(4-morpholinyl)acetamide;

N-(6-{6-chloro-5-[(phenylsulfonyl)amino]-3-pyridinyl}-1H-indazol-4-yl)-2-(4-morpholinyl)acetamide;

N-[6-(6-fluoro-1H-indol-4-yl)-1H-indazol-4-yl]-2-(4-morpholinyl)acetamide;

N-[3-fluoro-6-(1H-pyrazolo[3,4-b]pyridin-5-yl)-1H-indazol-4-yl]-2-(4-morpholinyl)acetamide;

N-[3-fluoro-6-(1H-indol-4-yl)-1H-indazol-4-yl]-2-(4-morpholinyl)acetamide; or

a salt thereof.

In a further embodiment, the compound of the invention is:

2-[(8aS)-hexahydropyrrolo[1,2-a]pyrazin-2(1H)-ylmethyl]-N-[6-(1H-indol-4-yl)-1H-indazol-4-yl]-1,3-thiazole-4-carboxamide;

2-{[(3R,5S)-3,5-dimethyl-4-(1-methylethyl)-1-piperazinyl]methyl}-N-[6-(1H-indol-4-yl)-1H-indazol-4-yl]-1,3-thiazole-4-carboxamide;

2-{[(3R,5S)-3,5-dimethyl-4-(1-methylethyl)-1-piperazinyl]methyl}-N-(6-{6-(methyloxy)-5-[(methylsulfonyl)amino]-3-pyridinyl}-1H-indazol-4-yl)-1,3-thiazole-4-carboxamide;

N-(6-{6-(methyloxy)-5-[(methylsulfonyl)amino]-3-pyridinyl}-1H-indazol-4-yl)-1-(tetrahydro-2H-pyran-4-ylmethyl)-1H-pyrazole-3-carboxamide;

N-[6-(1H-indol-4-yl)-1H-indazol-4-yl]-1-(tetrahydro-2H-pyran-4-ylmethyl)-1H-pyrazole-3-carboxamide; or

a salt thereof.

Terms and Definitions

“Alkyl” refers to a saturated hydrocarbon chain having the specified number of member atoms. For example, C₁₋₆alkyl refers to an alkyl group having from 1 to 6 member atoms. Alkyl groups may be optionally substituted with one or more substituents if so defined herein. Alkyl groups may be straight or branched. Representative branched alkyl groups have one, two, or three branches. Alkyl includes methyl, ethyl, propyl (n-propyl and isopropyl), butyl (n-butyl, isobutyl, and t-butyl), pentyl (n-pentyl, isopentyl, and neopentyl), and hexyl.

“Cycloalkyl” refers to a saturated hydrocarbon ring having the specified number of member atoms. Cycloalkyl groups are monocyclic ring systems. For example, C₃₋₆cycloalkyl refers to a cycloalkyl group having from 3 to 6 member atoms. Cycloalkyl includes cyclopropyl, cyclobutyl, cyclopentyl, and cyclohexyl.

“Enantiomerically enriched” refers to products whose enantiomeric excess is greater than zero. For example, enantiomerically enriched refers to products whose enantiomeric excess is greater than 50% ee, greater than 75% ee, and greater than 90% ee.

“Enantiomeric excess” or “ee” is the excess of one enantiomer over the other expressed as a percentage. As a result, since both enantiomers are present in equal amounts in a racemic mixture, the enantiomeric excess is zero (0% ee). However, if one enantiomer was enriched such that it constitutes 95% of the product, then the enantiomeric excess would be 90% ee (the amount of the enriched enantiomer, 95%, minus the amount of the other enantiomer, 5%).

“Enantiomerically pure” refers to products whose enantiomeric excess is 99% ee or greater.

“Half-life” (or “half-lives”) refers to the time required for half of a quantity of a substance to be converted to another chemically distinct species in vitro or in vivo.

“Halo” refers to the halogen radical fluoro, chloro, bromo, or iodo.

“Heteroaryl”, unless otherwise defined, refers to an aromatic ring containing from 1 to 3 heteroatoms as member atoms in the ring. Heteroaryl groups containing more than one heteroatom may contain different heteroatoms. Heteroaryl groups may be optionally substituted with one or more substituents if so defined herein. The heteroaryl groups herein are monocyclic ring systems or are fused bicyclic ring systems. Monocyclic heteroaryl rings have 5 member atoms. Bicyclic heteroaryl rings have 9 or 10 member atoms. Monocyclic heteroaryl includes pyrrolyl, furanyl, thienyl, pyrazolyl, imidazolyl, oxazolyl, isoxazolyl, thiazolyl, isothiazolyl, furazanyl, triazolyl and thiadiazolyl. In one embodiment, monocyclic heteroaryl is pyrazolyl or thiazolyl. Bicyclic heteroaryl includes indolyl, isoindolyl, indolizinyl, benzofuranyl, isobenzofuranyl, indazolyl, purinyl, benzimidazolyl, pyrrolopyridinyl, pyrazolopyridinyl, pyrrolopyrimidinyl, quinolyl, isoquinolinyl, quinoxalinyl, quinazolinyl, cinnolinyl, benzopyranyl, benzoxazolyl, furopyridinyl and naphthridinyl. In one embodiment, bicyclic heteroaryl is indolyl or pyrazolopyridinyl.

“Heteroatom” refers to a nitrogen, sulphur, or oxygen atom.

“Heterocyclyl”, unless otherwise defined, refers to a saturated or unsaturated ring containing 1 or 2 heteroatoms as member atoms in the ring. However, heterocyclyl rings are not aromatic. In certain embodiments, heterocyclyl is saturated. In other embodiments, heterocyclyl is unsaturated but not aromatic. Heterocyclyl groups may be optionally substituted with one or more substituents if so defined herein. The heterocyclyl groups herein may be monocyclic ring systems having 6 member atoms. Monocyclic heterocyclyl includes piperidinyl, piperazinyl and morpholinyl. The heterocyclyl groups herein may be bicyclic systems having 9 member atoms. Bicyclic heterocycyl groups include those rings wherein a monocyclic heterocyclyl and a monocyclic cycloalkyl or a further monocyclic heterocyclyl are attached forming a fused bicyclic ring system. Bicyclic heterocyclyl includes hexahydropyrrolo[1,2-a]pyrazinyl.

“Member atoms” refers to the atom or atoms that form a chain or ring. Where more than one member atom is present in a chain and within a ring, each member atom is covalently bound to an adjacent member atom in the chain or ring. Atoms that make up a substituent group on a chain or ring are not member atoms in the chain or ring.

“Optionally substituted” indicates that a group, such as heteroaryl, may be unsubstituted or substituted with one or more substituents if so defined herein.

“Substituted” in reference to a group indicates that a hydrogen atom attached to a member atom within a group is replaced. It should be understood that the term “substituted” includes the implicit provision that such substitution be in accordance with the permitted valence of the substituted atom and the substituent and that the substitution results in a stable compound (i.e. one that does not spontaneously undergo transformation such as by rearrangement, cyclization, or elimination). In certain embodiments, a single atom may be substituted with more than one substituent as long as such substitution is in accordance with the permitted valence of the atom. Suitable substituents are defined herein for each substituted or optionally substituted group.

“Pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

aq Aqueous

DCM Dichloromethane

DIBAL Diisobutylaluminium hydride

DIPEA Diisopropylethylamine

DMF N,N-Dimethylformamide

DMSO Dimethylsulfoxide

Et₃N Triethylamine

g Grams

h Hour(s)

HATU O-(7-Azabenzotriazol-1-yl)-N,N,N′,N′-tetramethyluronium hexafluorophosphate

HCl Hydrogen Chloride

HPLC High performance liquid chromatography

IPA Isopropanol

LCMS Liquid chromatography/mass spectroscopy

M Molar

MDAP Mass Directed Automated Preparative HPLC

MeOH Methanol

MeCN Acetonitrile

mg Milligrams

min Minutes

ml Millilitres

mmol Millimoles

mp Melting point

Pd(dppf)Cl₂ [1,1′-Bis(diphenylphosphino)ferrocene]dichloropalladium(II)

Pd(dppf)Cl₂—CH₂Cl₂ [1,1′-Bis(diphenylphosphino)ferrocene]dichloropalladium(II) dichloromethane adduct

Pd(PPh₃)₄ Tetrakis(triphenylphosphine)palladium(0)

Rₜ Retention time

RT room temperature

s Seconds

SCX Strong cation exchange

Solvias Catalyst chloro[2′-(dimethylamino)-2-biphenylyl]palladium-(1R,4S)-bicyclo[2.2.1]hept-2-yl[(1S,4R)-bicyclo[2.2.1]hept-2-yl]phosphane (1:1)

SPE Solid Phase Extraction

THF Tetrahydrofuran

TFA Trifluoroacetic acid

All references to brine are to a saturated aqueous solution of NaCl.

Included within the scope of the “compounds of the invention” are all solvates (including hydrates), complexes, polymorphs, prodrugs, radiolabelled derivatives, stereoisomers and optical isomers of the compounds of formula (I) and salts thereof.

The compounds of the invention may exist in solid or liquid form. In the solid state, the compounds of the invention may exist in crystalline or noncrystalline form, or as a mixture thereof. For compounds of the invention that are in crystalline form, the skilled artisan will appreciate that pharmaceutically acceptable solvates may be formed wherein solvent molecules are incorporated into the crystalline lattice during crystallization. Solvates may involve nonaqueous solvents such as ethanol, isopropanol, DMSO, acetic acid, ethanolamine, and EtOAc, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

The skilled artisan will further appreciate that certain compounds of the invention that exist in crystalline form, including the various solvates thereof, may exhibit polymorphism (i.e.

the capacity to occur in different crystalline structures). These different crystalline forms are typically known as “polymorphs”. The invention includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making or recrystallising the compound. For example, changes in temperature, pressure, or solvent may result in polymorphs. In addition, one polymorph may spontaneously convert to another polymorph under certain conditions.

The invention also includes isotopically-labelled compounds, which are identical to the compounds of formula (I) and salts thereof, but for the fact that one or more atoms are replaced by an atom having an atomic mass or mass number different from the atomic mass or mass number most commonly found in nature. Examples of isotopes that can be incorporated into the compounds of the invention include isotopes of hydrogen, carbon, nitrogen, oxygen and fluorine, such as 3H, 11C, 14C and 18F.

The compounds according to formula (I) may contain one or more asymmetric center (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in formula (I), or in any chemical structure illustrated herein, is not specified the structure is intended to encompass any stereoisomer and all mixtures thereof. Thus, compounds according to formula (I) containing one or more chiral center may be used as racemic mixtures, enantiomerically enriched mixtures, or as enantiomerically pure individual stereoisomers.

Individual stereoisomers of a compound according to formula (I) which contain one or more asymmetric center may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral enviornment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

The compounds according to formula (I) may also contain centers of geometric asymmetry. Where the stereochemistry of a center of geometric asymmetry present in formula (I), or in any chemical structure illustrated herein, is not specified, the structure is intended to encompass the trans geometric isomer, the cis geometric isomer, and all mixtures thereof. Likewise, all tautomeric forms are also included in formula (I) whether such tautomers exist in equilibrium or predominately in one form.

It is to be understood that the references herein to compounds of formula (I) and salts thereof covers the compounds of formula (I) as free acids or free bases, or as salts thereof, for example as pharmaceutically acceptable salts thereof. Thus, in one embodiment, the invention is directed to compounds of formula (I) as the free acid or free base. In another embodiment, the invention is directed to compounds of formula (I) and salts thereof. In a further embodiment, the invention is directed to compounds of formula (I) and pharmaceutically acceptable salts thereof.

The skilled artisan will appreciate that pharmaceutically acceptable salts of the compounds according to formula (I) may be prepared. Indeed, in certain embodiments of the invention, pharmaceutically acceptable salts of the compounds according to formula (I) may be preferred over the respective free base or free acid because such salts impart greater stability or solubility to the molecule thereby facilitating formulation into a dosage form. Accordingly, the invention is further directed to compounds of formula (I) and pharmaceutically acceptable salts thereof.

As used herein, the term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively.

Salts and solvates having non-pharmaceutically acceptable counter-ions or associated solvents are within the scope of the present invention, for example, for use as intermediates in the preparation of other compounds of formula (I) and their pharmaceutically acceptable salts. Thus one embodiment of the invention embraces compounds of formula (I) and salts thereof.

In certain embodiments, compounds according to formula (I) may contain an acidic functional group. Suitable pharmaceutically-acceptable salts include salts of such acidic functional groups. Representative salts include pharmaceutically acceptable metal salts such as sodium, potassium, lithium, calcium, magnesium, aluminum, and zinc salts; carbonates and bicarbonates of a pharmaceutically acceptable metal cation such as sodium, potassium, lithium, calcium, magnesium, aluminum, and zinc; pharmaceutically acceptable organic primary, secondary, and tertiary amines including aliphatic amines, aromatic amines, aliphatic diamines, and hydroxy alkylamines such as methylamine, ethylamine, 2-hydroxyethylamine, diethylamine, TEA, ethylenediamine, ethanolamine, diethanolamine, and cyclohexylamine.

In certain embodiments, compounds according to formula (I) may contain a basic functional group and are therefore capable of forming pharmaceutically acceptable acid addition salts by treatment with a suitable acid. Suitable acids include pharmaceutically acceptable inorganic acids and pharmaceutically acceptable organic acids. Representative pharmaceutically acceptable acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, naphthoate, hydroxynaphthoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), and napthalene-2-sulfonate.

Compound Preparation

The compounds of the invention may be made by a variety of methods, including standard chemistry. Any previously defined variable will continue to have the previously defined meaning unless otherwise indicated. Illustrative general synthetic methods are set out below and then specific compounds of the invention are prepared in the Examples section.

Process a

Compounds of formula (I) wherein R¹ and R³ are as defined above and R² is —NHCOR⁶ wherein R⁶ is 5-membered heteroaryl wherein the 5-membered heteroaryl contains from one to three heteroatoms independently selected from oxygen, nitrogen and sulphur and is substituted by —CH₂NR⁸R⁹, and salts thereof, may be prepared from compounds of formula (II)

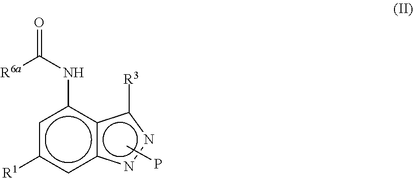

wherein R¹ and R³ are as defined above, R^{6a} is 5-membered heteroaryl wherein the 5-membered heteroaryl contains from one to three heteroatoms independently selected from oxygen, nitrogen and sulphur and is substituted by —CH₂X, wherein X is a leaving group, for example Cl, and wherein P is a protecting group, for example benzenesulphonyl, by a process comprising treatment with an amine of formula NHR⁸R⁹ in the presence of a suitable base such as DIPEA, a suitable activating agent such as sodium iodide and in a suitable solvent such as acetonitrile, heating to a suitable temperature such as from 20° C. to 120° C., for example about 70° C.

As the skilled person will appreciate, in the compound of formula (II), the protecting group P may be on the 1 or 2 position of the indazole. Following reaction with the amine, the protecting group P may be removed by deprotection under appropriate conditions.

Compounds of formula (II) wherein R¹ and R³ are as defined above, R^{6a} is 5-membered heteroaryl wherein the 5-membered heteroaryl contains from one to three heteroatoms independently selected from oxygen, nitrogen and sulphur and is substituted by —CH₂X, wherein X is a leaving group, for example Cl, and wherein P is a protecting group, for example benzenesulphonyl, may be prepared from compounds of formula (III)

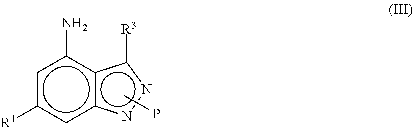

wherein R¹, R³ and P are as defined above, by a process comprising treatment with an acid chloride of formula R^{6a}COCl, wherein R^{6a} is as defined above, in the presence of a suitable base such as pyridine, in a suitable solvent such as DCM, and at a suitable temperature such as room temperature.

Compounds of formula R^{6a}COCl, wherein R^{6a} is as defined above, can be prepared from compounds of formula R^{6a}CO₂H, wherein R^{6a} is as defined above, by treatment with thionyl chloride in a suitable solvent such as chloroform, in the presence of DMF (catalytic quantity) and heating to a suitable temperature such as reflux.

Compounds of formula (III) wherein R¹, R³ and P are as defined above may be prepared from compounds of formula (IV)

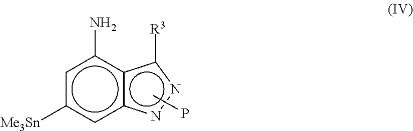

wherein R³ and P are as defined above, by treatment with a suitable halide, in the presence of a suitable palladium catalyst such as tetrakis(triphenylphosphine) palladium (0), in a suitable solvent such as N,N-dimethylformamide, and at a suitable temperature such as from 80 to 150° C., for example about 120° C.

Compounds of formula (IV) wherein R³ and P are as defined above, may be prepared from compounds of formula (V)

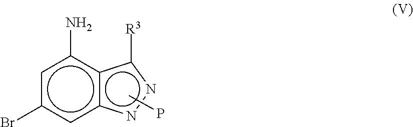

wherein R³ and P are as defined above, by treatment with a suitable stannane such as hexamethyldistannane, under microwave irradiation, in the presence of a suitable palladium catalyst such as tetrakis(triphenylphosphine)palladium (0), in a suitable solvent such as toluene, in the presence of a suitable base such as triethylamine, and at a suitable temperature such as from 80 to 150° C., for example about 120° C.

Alternatively, compounds of formula (III) wherein R¹, R³ and P are as defined above may be prepared from compounds of formula (V) as defined above by treatment with a suitable boronate such as 4-(4,4,5,5-tetramethyl-1,3,2-dioxaborolan-2-yl)-1H-indole, under microwave irradiation, in the presence of a suitable palladium catalyst such as 1,1′-bis(diphenylphosphino)ferrocene palladium dichloride, in a suitable solvent such as 1,4-dioxane, in the presence of a suitable base such as sodium carbonate, and at a suitable temperature such as from 60 to 150° C., for example about 100° C.

Process b

In an alternative approach, compounds of formula (I) wherein R³ is as defined above, R¹ is pyridinyl substituted by —NHSO₂R⁵ and further optionally substituted by a substituent R¹² which is C₁₋₆alkyl, —OR⁴ or halo, and R² is —NHCOR⁶, and salts thereof, may be prepared from compounds of formula (VI)

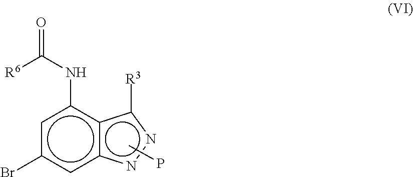

wherein R³ and R⁶ are as defined above and P is a protecting group, by treatment with a boronic ester of formula (VII)

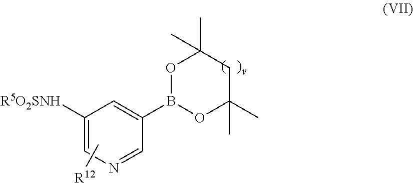

wherein R¹² and R⁵ are as defined above and v is 0 or 1, under microwave irradiation, in the presence of a suitable palladium catalyst such as 1,1′-bis(diphenylphosphino)ferrocene palladium dichloride, in a suitable solvent such as aqueous 1,4-dioxane, in the presence of a suitable base such as sodium carbonate, and at a suitable temperature such as from 60 to 150° C., for example about 120° C., followed by deprotection.

Compounds of formula (VII) wherein R⁵ and R¹² are as defined above, and salts thereof, can be prepared from compounds of formula (VIII)

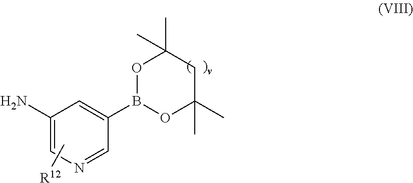

wherein R¹² and v are as defined above, by treatment with a sulphonyl chloride such as methanesulfonyl chloride in the presence of a suitable base such as pyridine, and at a suitable temperature such as room temperature.

Compounds of formula (VIII) wherein R¹² and v are as defined above, may be prepared from compounds of formula (IX) wherein R¹² is as defined above, for which a range of analogues are commercially available.

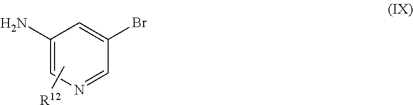

Process c

Compounds of formula (I) wherein R¹ and R³ are as defined above, and R² is —NHCOR⁶ wherein R⁶ is as defined above, and salts thereof, may also be prepared from compounds of formula (X)

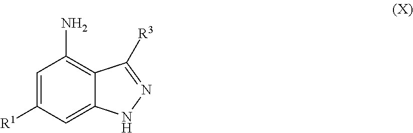

wherein R¹ and R³ are as defined above, by a process comprising treatment with an acid of formula R⁶COOH, wherein R⁶ is as defined above.

Suitable conditions include stirring in a suitable solvent such as N,N-dimethylformamide, at a suitable temperature such as room temperature, for example about 20° C., in the presence of a coupling reagent such as O-(7-azabenzotriazol-1-yl)-N,N,N′N′-tetramethyluronium hexafluorophosphate, and in the presence of a suitable base such as N,N-diisopropylethylamine.

Compounds of formula (X) wherein R¹ and R³ are as defined above, may be prepared from the compound of formula (XI)

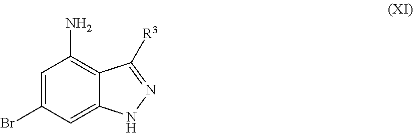

wherein R³ is H, by treatment with a suitable boronic acid or boronate ester such as 4-(4,4,5,5-tetramethyl-1,3,2-dioxaborolan-2-yl)-1H-indole (commercially available), in the presence of a suitable palladium catalyst such as 1,1′-bis(diphenylphosphino)ferrocene palladium dichloride, in a suitable solvent such as a mixture of 1,4-dioxane and water, in the presence of a suitable base such as sodium carbonate, and at a suitable temperature such as from 60 to 200° C., for example about 115° C. Alternatively, this process may be carried out under microwave irradiation, at a suitable temperature such as from 60 to 200° C., for example about 150° C.

Process d

Compounds of formula (I) wherein R¹ and R³ are as defined above and R² is

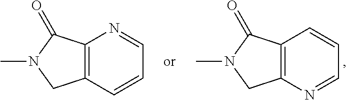

and salts thereof, may be prepared from compounds of formula (X) as defined above, by treatment with the compound of formula (XII)

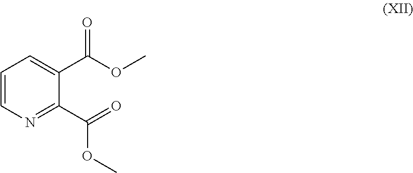

Suitable conditions include stirring in a suitable solvent such as toluene, at a suitable temperature such as about -65° C., and in the presence of a suitable base such as DIBAL.

Process e

Compounds of formula (I) wherein R¹, R² and R³ are as defined above, and salts thereof, may also be prepared by a process comprising deprotection of suitably protected derivatives of compounds of formula (IA) wherein R¹, R² and R³ are as defined above and P is a protecting group. Examples of suitable protection groups and the means of their removal can be found in T. W. Greene and P. G. M. Wuts ‘Protective Groups in Organic Synthesis’ (3^{rd} Ed., J. Wiley and Sons, 1999).

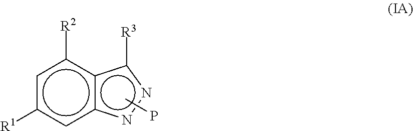

As an example of this, compounds of formula (I) may be prepared from compounds of formula (IA) where the indazole ring nitrogen is protected (P), for example with 1-phenylsulphonyl, by deprotection under appropriate conditions, such as treating with a base, for example aqueous sodium hydroxide.

Compounds of formula (IA), wherein R¹, R² and R³ are as defined above, may be prepared from compounds of formula (XIII)

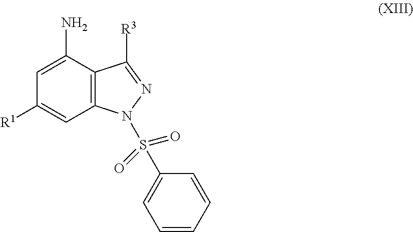

wherein R¹ and R³ are as defined above, by treatment with an acid of formula R⁶COOH, wherein R² is as defined above. Suitable conditions include stirring in a suitable solvent such as N,N-dimethylformamide, at a suitable temperature such as room temperature, for example about 20° C., in the presence of a coupling reagent such as O-(7-azabenzotriazol-1-yI)-N,N,N′N′-tetramethyluronium hexafluorophosphate, and in the presence of a suitable base such as N,N-diisopropylethylamine.

Thus, in one embodiment, the invention provides a process for preparing a compound of the invention comprising:

a) for a compound of formula (I) wherein R¹ and R³ are as defined above, and R² is —NHCOR⁶ wherein R⁶ is 5-membered heteroaryl wherein the 5-membered heteroaryl contains from one to three heteroatoms independently selected from oxygen, nitrogen and sulphur and is substituted by —CH₂NR⁸R⁹, and salts thereof, reacting a compound of formula (II)

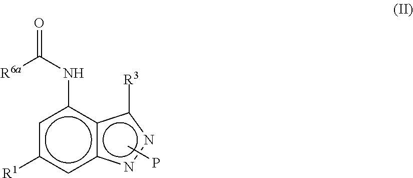

wherein R¹ and R³ are as defined above, R^{6a} is 5-membered heteroaryl wherein the 5-membered heteroaryl contains from one to three heteroatoms independently selected from oxygen, nitrogen and sulphur and is substituted by —CH₂X, wherein X is a leaving group, and wherein P is a protecting group, with an amine of formula NHR⁸R⁹, followed by deprotection;

b) for a compound of formula (I) wherein R³ is as defined above, R¹ is pyridinyl substituted by —NHSO₂R⁵ and further optionally substituted by a substituent R¹² which is C₁₋₆alkyl, —OR⁴ or halo, and R² is —NHCOR⁶, and salts thereof, reacting a compound of formula (VI)

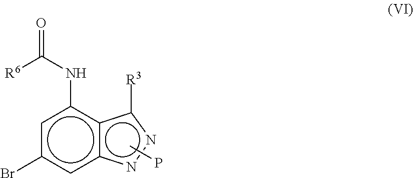

wherein R³ and R⁶ are as defined above and P is a protecting group, with a boronic ester of formula (VII)

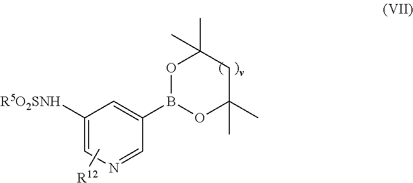

wherein R¹² and R⁵ are as defined above and v is 0 or 1, followed by deprotection;

c) for a compound of formula (I) wherein R¹ and R³ are as defined above, and R² is —NHCOR⁶ wherein R⁶ is as defined above, and salts thereof, reacting a compound of formula (X)

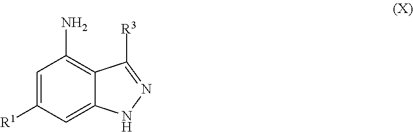

wherein R¹ and R³ are as defined above, with an acid of formula R⁶COOH, wherein R⁶ is as defined above;

d) for a compound of formula (I) wherein R¹ and R³ are as defined above and R² is

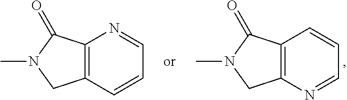

and salts thereof, reacting a compound of formula (X) as defined above, by treatment with the compound of formula (XII)

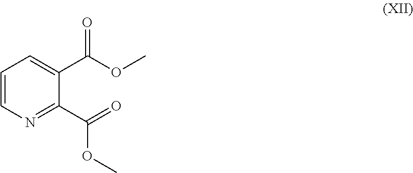

; or

e) deprotection of a suitably protected derivative of a compound of formula (IA)

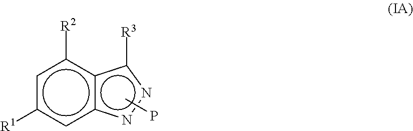

wherein R¹, R² and R³ are as defined above and P is a protecting group.

Methods of Use

The compounds of the invention are inhibitors of PI3-kinase activity. Compounds which are PI3-kinase inhibitors may be useful in the treatment of disorders wherein the underlying pathology is (at least in part) attributable to inappropriate PI3-kinase activity, such as asthma and chronic obstructive pulmonary disease (COPD). “Inappropriate PI3-kinase activity” refers to any PI3-kinase activity that deviates from the normal PI3-kinase activity expected in a particular patient. Inappropriate PI3-kinase may take the form of, for instance, an abnormal increase in activity, or an aberration in the timing and or control of PI3-kinase activity. Such inappropriate activity may result then, for example, from overexpression or mutation of the protein kinase leading to inappropriate or uncontrolled activation. Accordingly, in another aspect the invention is directed to methods of treating such disorders.

Such disorders include respiratory diseases including asthma and chronic obstructive pulmonary disease (COPD); allergic diseases including allergic rhinitis and atopic dermatitis; autoimmune diseases including rheumatoid arthritis and multiple sclerosis; inflammatory disorders including inflammatory bowel disease; cardiovascular diseases including thrombosis and atherosclerosis; hematologic malignancies; cystic fibrosis; neurodegenerative diseases; pancreatitis; multiorgan failure; kidney diseases; platelet aggregation; cancer; sperm motility; transplantation rejection; graft rejection; lung injuries; and pain including pain associated with rheumatoid arthritis or osteoarthritis, back pain, general inflammatory pain, post hepatic neuralgia, diabetic neuropathy, inflammatory neuropathic pain (trama), trigeminal neuralgia and central pain.

The methods of treatment of the invention comprise administering a safe and effective amount of a compound of formula (I) or a pharmaceutically acceptable salt thereof to a patient in need thereof. Individual embodiments of the invention include methods of treating any one of the above-mentioned disorders by administering a safe and effective amount of a compound of formula (I) or a pharmaceutically acceptable salt thereof to a patient in need thereof.

As used herein, “treat” in reference to a disorder means: (1) to ameliorate or prevent the disorder or one or more of the biological manifestations of the disorder, (2) to interfere with (a) one or more points in the biological cascade that leads to or is responsible for the disorder or (b) one or more of the biological manifestations of the disorder, (3) to alleviate one or more of the symptoms or effects associated with the disorder, or (4) to slow the progression of the disorder or one or more of the biological manifestations of the disorder.

As indicated above, “treatment” of a disorder includes prevention of the disorder. The skilled artisan will appreciate that “prevention” is not an absolute term. In medicine, “prevention” is understood to refer to the prophylactic administration of a drug to substantially diminish the likelihood or severity of a disorder or biological manifestation thereof, or to delay the onset of such disorder or biological manifestation thereof.

As used herein, “safe and effective amount” in reference to a compound of formula (I) or a pharmaceutically acceptable salt thereof or other pharmaceutically-active agent means an amount of the compound sufficient to treat the patient's condition but low enough to avoid serious side effects (at a reasonable benefit/risk ratio) within the scope of sound medical judgment. A safe and effective amount of a compound will vary with the particular compound chosen (e.g. consider the potency, efficacy, and half-life of the compound); the route of administration chosen; the disorder being treated; the severity of the disorder being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

As used herein, “patient” refers to a human (including adults and children) or other animal. In one embodiment, “patient” refers to a human.

The compounds of formula (I) or pharmaceutically acceptable salts thereof may be administered by any suitable route of administration, including both systemic administration and topical administration. Systemic administration includes oral administration, parenteral administration, transdermal administration and rectal administration. Parenteral administration refers to routes of administration other than enteral or transdermal, and is typically by injection or infusion. Parenteral administration includes intravenous, intramuscular, and subcutaneous injection or infusion. Topical administration includes application to the skin as well as intraocular, otic, intravaginal, inhaled and intranasal administration. Inhalation refers to administration into the patient's lungs whether inhaled through the mouth or through the nasal passages. In one embodiment, the compounds of formula (I) or pharmaceutically acceptable salts thereof may be administered orally. In another embodiment, the compounds of formula (I) or pharmaceutically acceptable salts thereof may be administered by inhalation. In a further embodiment, the compounds of formula (I) or pharmaceutically acceptable salts thereof may be administered intranasally.

The compounds of formula (I) or pharmaceutically acceptable salts thereof may be administered once or according to a dosing regimen wherein a number of doses are administered at varying intervals of time for a given period of time. For example, doses may be administered one, two, three, or four times per day. In one embodiment, a dose is administered once per day. In a further embodiment, a dose is administered twice per day. Doses may be administered until the desired therapeutic effect is achieved or indefinitely to maintain the desired therapeutic effect. Suitable dosing regimens for a compound of formula (I) or a pharmaceutically acceptable salt thereof depend on the pharmacokinetic properties of that compound, such as absorption, distribution, and half-life, which can be determined by the skilled artisan. In addition, suitable dosing regimens, including the duration such regimens are administered, for a compound of formula (I) or a pharmaceutically acceptable salt thereof depend on the disorder being treated, the severity of the disorder being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Typical daily dosages may vary depending upon the particular route of administration chosen. Typical daily dosages for oral administration range from 0.001 mg to 50 mg per kg of total body weight, for example from 1 mg to 10 mg per kg of total body weight. For example, daily dosages for oral administration may be from 0.5 mg to 2 g per patient, such as 10 mg to 1 g per patient.

Additionally, the compounds of formula (I) may be administered as prodrugs. As used herein, a “prodrug” of a compound of formula (I) is a functional derivative of the compound which, upon administration to a patient, eventually liberates the compound of formula (I) in vivo. Administration of a compound of formula (I) as a prodrug may enable the skilled artisan to do one or more of the following: (a) modify the onset of the activity of the compound in vivo; (b) modify the duration of action of the compound in vivo; (c) modify the transportation or distribution of the compound in vivo; (d) modify the solubility of the compound in vivo; and (e) overcome a side effect or other difficulty encountered with the compound. Typical functional derivatives used to prepare prodrugs include modifications of the compound that are chemically or enzymatically cleavable in vivo. Such modifications, which include the preparation of phosphates, amides, esters, thioesters, carbonates, and carbamates, are well known to those skilled in the art.

The invention thus provides a method of treating a disorder mediated by inappropriate PI3-kinase activity comprising administering a safe and effective amount of a compound of formula (I) or a pharmaceutically acceptable salt thereof to a patient in need thereof.

In one embodiment, the disorder mediated by inappropriate PI3-kinase activity is selected from the group consisting of respiratory diseases (including asthma and chronic obstructive pulmonary disease (COPD)); allergic diseases (including allergic rhinitis and atopic dermatitis); autoimmune diseases (including rheumatoid arthritis and multiple sclerosis); inflammatory disorders (including inflammatory bowel disease); cardiovascular diseases (including thrombosis and atherosclerosis); hematologic malignancies; cystic fibrosis; neurodegenerative diseases; pancreatitis; multiorgan failure; kidney diseases; platelet aggregation; cancer; sperm motility; transplantation rejection; graft rejection; lung injuries; and pain (including pain associated with rheumatoid arthritis or osteoarthritis, back pain, general inflammatory pain, post hepatic neuralgia, diabetic neuropathy, inflammatory neuropathic pain (trama), trigeminal neuralgia and central pain).

In one embodiment, the disorder mediated by inappropriate PI3-kinase activity is a respiratory disease. In a further embodiment, the disorder mediated by inappropriate PI3-kinase activity is asthma. In a further embodiment, the disorder mediated by inappropriate PI3-kinase activity is chronic obstructive pulmonary disease (COPD).

In one embodiment, the disorder mediated by inappropriate PI3-kinase activity is pain.

In one embodiment, the invention provides a compound of formula (I) or a pharmaceutically acceptable salt thereof for use in medical therapy. In another embodiment, the invention provides a compound of formula (I) or a pharmaceutically acceptable salt thereof for use in the treatment of a disorder mediated by inappropriate PI3-kinase activity. In a further embodiment, the invention provides the use of a compound of formula (I) or a pharmaceutically acceptable salt thereof in the manufacture of a medicament for use in the treatment of a disorder mediated by inappropriate PI3-kinase activity.

Compositions

The compounds of formula (I) and pharmaceutically acceptable salts thereof will normally, but not necessarily, be formulated into pharmaceutical compositions prior to administration to a patient. Accordingly, in another aspect the invention is directed to pharmaceutical compositions comprising a compound of formula (I) or a pharmaceutically acceptable salt thereof and one or more pharmaceutically-acceptable excipients.

The pharmaceutical compositions of the invention may be prepared and packaged in bulk form wherein a safe and effective amount of a compound of formula (I) or a pharmaceutically acceptable salt thereof can be extracted and then given to the patient such as with powders or syrups. Alternatively, the pharmaceutical compositions of the invention may be prepared and packaged in unit dosage form wherein each physically discrete unit contains a compound of formula (I) or a pharmaceutically acceptable salt thereof. When prepared in unit dosage form, the pharmaceutical compositions of the invention typically may contain, for example, from 0.5 mg to 1 g, or from 1 mg to 700 mg, or from 5 mg to 100 mg of a compound of formula (I) or a pharmaceutically acceptable salt thereof.

The pharmaceutical compositions of the invention typically contain one compound of formula (I) or a pharmaceutically acceptable salt thereof.

As used herein, “pharmaceutically-acceptable excipient” means a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of the compound of formula (I) or a pharmaceutically acceptable salt thereof when administered to a patient and interactions which would result in pharmaceutical compositions that are not pharmaceutically acceptable are avoided. In addition, each excipient must of course be pharmaceutically-acceptable eg of sufficiently high purity.

The compound of formula (I) or a pharmaceutically acceptable salt thereof and the pharmaceutically-acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols, solutions, and dry powders; and (6) topical administration such as creams, ointments, lotions, solutions, pastes, sprays, foams, and gels.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting of the compound or compounds of formula (I) or pharmaceutically acceptable salts thereof once administered to the patient from one organ, or portion of the body, to another organ, or portion of the body. Certain pharmaceutically acceptable excipients may be chosen for their ability to enhance patient compliance.

Suitable pharmaceutically-acceptable excipients include the following types of excipients: Diluents, fillers, binders, disintegrants, lubricants, glidants, granulating agents, coating agents, wetting agents, solvents, co-solvents, suspending agents, emulsifiers, sweetners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other excipients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

Accordingly, in another aspect the invention is directed to process for the preparation of a pharmaceutical composition comprising a compound of formula (I) or a pharmaceutically acceptable salt thereof and one or more pharmaceutically-acceptable excipients which comprises mixing the ingredients. A pharmaceutical composition comprising a compound of formula (I) or a pharmaceutically acceptable salt thereof may be prepared by, for example, admixture at ambient temperature and atmospheric pressure.

In one embodiment, the compounds of formula (I) or pharmaceutically acceptable salts thereof will be formulated for oral administration. In another embodiment, the compounds of formula (I) or pharmaceutically acceptable salts thereof will be formulated for inhaled administration. In a further embodiment, the compounds of formula (I) or pharmaceutically acceptable salts thereof will be formulated for intranasal administration. In one aspect, the invention is directed to a solid oral dosage form such as a tablet or capsule comprising a safe and effective amount of a compound of formula (I) or a pharmaceutically acceptable salt thereof and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesuim stearate, calcium stearate, and talc.

Where appropriate, dosage unit formulations for oral administration can be microencapsulated. The composition can also be prepared to prolong or sustain the release as for example by coating or embedding particulate material in polymers, wax or the like.

The compounds of formula (I) or pharmaceutically acceptable salts thereof may also be coupled with soluble polymers as targetable drug carriers. Such polymers can include polyvinylpyrrolidone, pyran copolymer, polyhydroxypropylmethacrylamide -phenol, polyhydroxyethylaspartamidephenol, or polyethyleneoxidepolylysine substituted with palmitoyl residues. Furthermore, the compounds of formula (I) or pharmaceutically acceptable salts thereof may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

In another aspect, the invention is directed to a liquid oral dosage form. Oral liquids such as solution, syrups and elixirs can be prepared in dosage unit form so that a given quantity contains a predetermined amount of a compound of formula (I) or a pharmaceutically acceptable salt thereof. Syrups can be prepared by dissolving the compound of formula (I) or a pharmaceutically acceptable salt thereof in a suitably flavored aqueous solution, while elixirs are prepared through the use of a non-toxic alcoholic vehicle. Suspensions can be formulated by dispersing the compound of formula (I) or a pharmaceutically acceptable salt thereof in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

In another aspect, the invention is directed to a dosage form adapted for administration to a patient by inhalation, for example, as a dry powder, an aerosol, a suspension, or a solution composition. For example, the invention is directed to a dry powder composition adapted for inhalation comprising compound of formula (I) or a pharmaceutically acceptable salt thereof.

Dry powder compositions for delivery to the lung by inhalation typically comprise a compound of formula (I) or a pharmaceutically acceptable salt thereof as a finely divided powder together with one or more pharmaceutically-acceptable excipients as finely divided powders. Pharmaceutically-acceptable excipients particularly suited for use in dry powders are known to those skilled in the art and include lactose, starch, mannitol, and mono-, di-, and polysaccharides. The finely divided powder may be prepared by, for example, micronisation and milling. Generally, the size-reduced (eg micronised) compound can be defined by a D₅₀ value of about 1 to about 10 microns (for example as measured using laser diffraction).

The dry powder may be administered to the patient via a reservoir dry powder inhaler (RDPI) having a reservoir suitable for storing multiple (un-metered doses) of medicament in dry powder form. RDPIs typically include a means for metering each medicament dose from the reservoir to a delivery position. For example, the metering means may comprise a metering cup, which is movable from a first position where the cup may be filled with medicament from the reservoir to a second position where the metered medicament dose is made available to the patient for inhalation.

Alternatively, the dry powder may be presented in capsules (e.g. gelatin or plastic), cartridges, or blister packs for use in a multi-dose dry powder inhaler (MDPI). MDPIs are inhalers wherein the medicament is comprised within a multi-dose pack containing (or otherwise carrying) multiple defined doses (or parts thereof) of medicament. When the dry powder is presented as a blister pack, it comprises multiple blisters for containment of the medicament in dry powder form. The blisters are typically arranged in regular fashion for ease of release of the medicament therefrom. For example, the blisters may be arranged in a generally circular fashion on a disc-form blister pack, or the blisters may be elongate in form, for example comprising a strip or a tape. Each capsule, cartridge, or blister may, for example, contain between 20 μg-10 mg of the compound of formula (I) or a pharmaceutically acceptable salt thereof.

Aerosols may be formed by suspending or dissolving a compound of formula (I) or a pharmaceutically acceptable salt thereof in a liquified propellant. Suitable propellants include halocarbons, hydrocarbons, and other liquified gases. Representative propellants include: trichlorofluoromethane (propellant 11), dichlorofluoromethane (propellant 12), dichlorotetrafluoroethane (propellant 114), tetrafluoroethane (HFA-134a), 1,1-difluoroethane (HFA-152a), difluoromethane (HFA-32), pentafluoroethane (HFA-12), heptafluoropropane (HFA-227a), perfluoropropane, perfluorobutane, perfluoropentane, butane, isobutane, and pentane. Aerosols comprising a compound of formula (I) or a pharmaceutically acceptable salt thereof will typically be administered to a patient via a metered dose inhaler (MDI). Such devices are known to those skilled in the art.

The aerosol may contain additional pharmaceutically-acceptable excipients typically used with MDIs such as surfactants, lubricants, cosolvents and other excipients to improve the physical stability of the formulation, to improve valve performance, to improve solubility, or to improve taste.

There is thus provided as a further aspect of the invention a pharmaceutical aerosol formulation comprising a compound of formula (I) or a pharmaceutically acceptable salt thereof and a fluorocarbon or hydrogen-containing chlorofluorocarbon as propellant, optionally in combination with a surfactant and/or a cosolvent.

According to another aspect of the invention, there is provided a pharmaceutical aerosol formulation wherein the propellant is selected from 1,1,1,2-tetrafluoroethane, 1,1,1,2,3,3,3-heptafluoro-n-propane and mixtures thereof.

The formulations of the invention may be buffered by the addition of suitable buffering agents.

Capsules and cartridges for use in an inhaler or insufflator, of for example gelatine, may be formulated containing a powder mix for inhalation of a compound of formula (I) or a pharmaceutically acceptable salt thereof and a suitable powder base such as lactose or starch. Each capsule or cartridge may generally contain from 20 μg to 10 mg of the compound of formula (I) or pharmaceutically acceptable salt thereof. Alternatively, the compound of formula (I) or pharmaceutically acceptable salt thereof may be presented without excipients such as lactose.

The proportion of the active compound of formula (I) or pharmaceutically acceptable salt thereof in the local compositions according to the invention depends on the precise type of formulation to be prepared but will generally be within the range of from 0.001 to 10% by weight. Generally, for most types of preparations, the proportion used will be within the range of from 0.005 to 1%, for example from 0.01 to 0.5%. However, in powders for inhalation or insufflation the proportion used will normally be within the range of from 0.1 to 5%.

Aerosol formulations are preferably arranged so that each metered dose or “puff” of aerosol contains from 20 μg to 10 mg, preferably from 20 μg to 2000 μg, more preferably from about 20 μg to 500 μg of a compound of formula (I). Administration may be once daily or several times daily, for example 2, 3, 4 or 8 times, giving for example 1, 2 or 3 doses each time. The overall daily dose with an aerosol will be within the range from 100 μg to 10 mg, preferably from 200 μg to 2000 μg. The overall daily dose and the metered dose delivered by capsules and cartridges in an inhaler or insufflator will generally be double that delivered with aerosol formulations.

In the case of suspension aerosol formulations, the particle size of the particulate (e.g., micronised) drug should be such as to permit inhalation of substantially all the drug into the lungs upon administration of the aerosol formulation and will thus be less than 100 microns, desirably less than 20 microns, and in particular in the range of from 1 to 10 microns, such as from 1 to 5 microns, more preferably from 2 to 3 microns.

The formulations of the invention may be prepared by dispersal or dissolution of the medicament and a compound of formula (I) or a pharmaceutically acceptable salt thereof in the selected propellant in an appropriate container, for example, with the aid of sonication or a high-shear mixer. The process is desirably carried out under controlled humidity conditions.

The chemical and physical stability and the pharmaceutical acceptability of the aerosol formulations according to the invention may be determined by techniques well known to those skilled in the art. Thus, for example, the chemical stability of the components may be determined by HPLC assay, for example, after prolonged storage of the product. Physical stability data may be gained from other conventional analytical techniques such as, for example, by leak testing, by valve delivery assay (average shot weights per actuation), by dose reproducibility assay (active ingredient per actuation) and spray distribution analysis.

The stability of the suspension aerosol formulations according to the invention may be measured by conventional techniques, for example, by measuring flocculation size distribution using a back light scattering instrument or by measuring particle size distribution by cascade impaction or by the “twin impinger” analytical process. As used herein reference to the “twin impinger” assay means “Determination of the deposition of the emitted dose in pressurised inhalations using apparatus A” as defined in British Pharmacopaeia 1988, pages A204-207, Appendix XVII C. Such techniques enable the “respirable fraction” of the aerosol formulations to be calculated. One method used to calculate the “respirable fraction” is by reference to “fine particle fraction” which is the amount of active ingredient collected in the lower impingement chamber per actuation expressed as a percentage of the total amount of active ingredient delivered per actuation using the twin impinger method described above.

The term “metered dose inhaler” or MDI means a unit comprising a can, a secured cap covering the can and a formulation metering valve situated in the cap. MDI system includes a suitable channelling device. Suitable channelling devices comprise for example, a valve actuator and a cylindrical or cone-like passage through which medicament may be delivered from the filled canister via the metering valve to the nose or mouth of a patient such as a mouthpiece actuator.

MDI canisters generally comprise a container capable of withstanding the vapour pressure of the propellant used such as a plastic or plastic-coated glass bottle or preferably a metal can, for example, aluminium or an alloy thereof which may optionally be anodised, lacquer-coated and/or plastic-coated (for example incorporated herein by reference WO96/32099 wherein part or all of the internal surfaces are coated with one or more fluorocarbon polymers optionally in combination with one or more non-fluorocarbon polymers), which container is closed with a metering valve. The cap may be secured onto the can via ultrasonic welding, screw fitting or crimping. MDIs taught herein may be prepared by methods of the art (e.g. see Byron, above and WO96/32099). Preferably the canister is fitted with a cap assembly, wherein a drug-metering valve is situated in the cap, and said cap is crimped in place.

In one embodiment of the invention the metallic internal surface of the can is coated with a fluoropolymer, more preferably blended with a non-fluoropolymer. In another embodiment of the invention the metallic internal surface of the can is coated with a polymer blend of polytetrafluoroethylene (PTFE) and polyethersulfone (PES). In a further embodiment of the invention the whole of the metallic internal surface of the can is coated with a polymer blend of polytetrafluoroethylene (PTFE) and polyethersulfone (PES).

The metering valves are designed to deliver a metered amount of the formulation per actuation and incorporate a gasket to prevent leakage of propellant through the valve. The gasket may comprise any suitable elastomeric material such as, for example, low density polyethylene, chlorobutyl, bromobutyl, EPDM, black and white butadiene-acrylonitrile rubbers, butyl rubber and neoprene. Suitable valves are commercially available from manufacturers well known in the aerosol industry, for example, from Valois, France (e.g. DF10, DF30, DF60), Bespak plc, UK (e.g. BK300, BK357) and 3M-Neotechnic Ltd, UK (e.g. Spraymiser™).

In various embodiments, the MDIs may also be used in conjunction with other structures such as, without limitation, overwrap packages for storing and containing the MDIs, including those described in U.S. Pat. Nos. 6,119,853; 6,179,118; 6,315,112; 6,352,152; 6,390,291; and 6,679,374, as well as dose counter units such as, but not limited to, those described in U.S. Pat. Nos. 6,360,739 and 6,431,168.

Conventional bulk manufacturing methods and machinery well known to those skilled in the art of pharmaceutical aerosol manufacture may be employed for the preparation of large-scale batches for the commercial production of filled canisters. Thus, for example, in one bulk manufacturing method for preparing suspension aerosol formulations a metering valve is crimped onto an aluminium can to form an empty canister. The particulate medicament is added to a charge vessel and liquefied propellant together with the optional excipients is pressure filled through the charge vessel into a manufacturing vessel. The drug suspension is mixed before recirculation to a filling machine and an aliquot of the drug suspension is then filled through the metering valve into the canister. In one example of a bulk manufacturing method for preparing solution aerosol formulations, a metering valve is crimped onto an aluminium can to form an empty canister. The liquefied propellant together with the optional excipients and the dissolved medicament is pressure filled through the charge vessel into a manufacturing vessel.

In an alternative process, an aliquot of the liquefied formulation is added to an open canister under conditions which are sufficiently cold to ensure the formulation does not vaporise, and then a metering valve crimped onto the canister.

Typically, in batches prepared for pharmaceutical use, each filled canister is check-weighed, coded with a batch number and packed into a tray for storage before release testing.

Suspensions and solutions comprising a compound of formula (I) or a pharmaceutically acceptable salt thereof may also be administered to a patient via a nebulizer. The solvent or suspension agent utilized for nebulization may be any pharmaceutically-acceptable liquid such as water, aqueous saline, alcohols or glycols, e.g., ethanol, isopropylalcohol, glycerol, propylene glycol, polyethylene glycol, etc. or mixtures thereof. Saline solutions utilize salts which display little or no pharmacological activity after administration. Both organic salts, such as alkali metal or ammonium halogen salts, e.g., sodium chloride, potassium chloride or organic salts, such as potassium, sodium and ammonium salts or organic acids, e.g., ascorbic acid, citric acid, acetic acid, tartaric acid, etc. may be used for this purpose.

Other pharmaceutically-acceptable excipients may be added to the suspension or solution. The compound of formula (I) or pharmaceutically acceptable salt thereof may be stabilized by the addition of an inorganic acid, e.g., hydrochloric acid, nitric acid, sulphuric acid and/or phosphoric acid; an organic acid, e.g., ascorbic acid, citric acid, acetic acid, and tartaric acid, etc., a complexing agent such as EDTA or citric acid and salts thereof; or an antioxidant such as antioxidant such as vitamin E or ascorbic acid. These may be used alone or together to stabilize the compound of formula (I) or pharmaceutically acceptable salt thereof. Preservatives may be added such as benzalkonium chloride or benzoic acid and salts thereof. Surfactant may be added particularly to improve the physical stability of suspensions. These include lecithin, disodium dioctylsulphosuccinate, oleic acid and sorbitan esters.

In a further aspect, the invention is directed to a dosage form adapted for intranasal administration.

Formulations for administration to the nose may include pressurised aerosol formulations and aqueous formulations administered to the nose by pressurised pump. Formulations which are non-pressurised and adapted to be administered topically to the nasal cavity are of particular interest. Suitable formulations contain water as the diluent or carrier for this purpose. Aqueous formulations for administration to the lung or nose may be provided with conventional excipients such as buffering agents, tonicity modifying agents and the like. Aqueous formulations may also be administered to the nose by nebulisation.

The compounds of formula (I) or pharmaceutically acceptable salts thereof may be formulated as a fluid formulation for delivery from a fluid dispenser, for example a fluid dispenser having a dispensing nozzle or dispensing orifice through which a metered dose of the fluid formulation is dispensed upon the application of a user-applied force to a pump mechanism of the fluid dispenser. Such fluid dispensers are generally provided with a reservoir of multiple metered doses of the fluid formulation, the doses being dispensable upon sequential pump actuations. The dispensing nozzle or orifice may be configured for insertion into the nostrils of the user for spray dispensing of the fluid formulation into the nasal cavity. A fluid dispenser of the aforementioned type is described and illustrated in WO05/044354, the entire content of which is hereby incorporated herein by reference. The dispenser has a housing which houses a fluid discharge device having a compression pump mounted on a container for containing a fluid formulation. The housing has at least one finger-operable side lever which is movable inwardly with respect to the housing to cam the container upwardly in the housing to cause the pump to compress and pump a metered dose of the formulation out of a pump stem through a nasal nozzle of the housing. In one embodiment, the fluid dispenser is of the general type illustrated in FIGS. 30-40 of WO05/044354.

Pharmaceutical compositions adapted for intranasal administration wherein the carrier is a solid include a coarse powder having a particle size for example in the range 20 to 500 microns which is administered by rapid inhalation through the nasal passage from a container of the powder held close up to the nose. Suitable compositions wherein the carrier is a liquid, for administration as a nasal spray or as nasal drops, include aqueous or oil solutions of the compound of formula (I) or a pharmaceutically acceptable salt thereof.

Pharmaceutical compositions adapted for transdermal administration may be presented as discrete patches intended to remain in intimate contact with the epidermis of the patient for a prolonged period of time. For example, the active ingredient may be delivered from the patch by iontophoresis as generally described in Pharmaceutical Research, 3(6), 318 (1986).

Pharmaceutical compositions adapted for topical administration may be formulated as ointments, creams, suspensions, lotions, powders, solutions, pastes, gels, sprays, aerosols or oils.

Ointments, creams and gels, may, for example, be formulated with an aqueous or oily base with the addition of suitable thickening and/or gelling agent and/or solvents. Such bases may thus, for example, include water and/or an oil such as liquid paraffin or a vegetable oil such as arachis oil or castor oil, or a solvent such as polyethylene glycol. Thickening agents and gelling agents which may be used according to the nature of the base include soft paraffin, aluminium stearate, cetostearyl alcohol, polyethylene glycols, woolfat, beeswax, carboxypolymethylene and cellulose derivatives, and/or glyceryl monostearate and/or non-ionic emulsifying agents.

Lotions may be formulated with an aqueous or oily base and will in general also contain one or more emulsifying agents, stabilising agents, dispersing agents, suspending agents or thickening agents.

Powders for external application may be formed with the aid of any suitable powder base, for example, talc, lactose or starch. Drops may be formulated with an aqueous or non-aqueous base also comprising one or more dispersing agents, solubilising agents, suspending agents or preservatives.

Topical preparations may be administered by one or more applications per day to the affected area; over skin areas occlusive dressings may advantageously be used. Continuous or prolonged delivery may be achieved by an adhesive reservoir system.

For treatments of the eye or other external tissues, for example mouth and skin, the compositions may be applied as a topical ointment or cream. When formulated in an ointment, the compound of formula (I) or a pharmaceutically acceptable salt thereof may be employed with either a paraffinic or a water-miscible ointment base. Alternatively, the compound of formula (I) or pharmaceutically acceptable salt thereof may be formulated in a cream with an oil-in-water cream base or a water-in-oil base.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

The compound and pharmaceutical formulations according to the invention may be used in combination with or include one or more other therapeutic agents, for example selected from anti-inflammatory agents, anticholinergic agents (particularly an M₁/M₂/M₃ receptor antagonist), β₂-adrenoreceptor agonists, antiinfective agents, such as antibiotics or antivirals, or antihistamines. The invention thus provides, in a further aspect, a combination comprising a compound of formula (I) or a pharmaceutically acceptable salt thereof together with one or more other therapeutically active agents, for example selected from an anti-inflammatory agent, such as a corticosteroid or an NSAID, an anticholinergic agent, a β₂-adrenoreceptor agonist, an antiinfective agent, such as an antibiotic or an antiviral, or an antihistamine. One embodiment of the invention encompasses combinations comprising a compound of formula (I) or a pharmaceutically acceptable salt thereof together with a β₂-adrenoreceptor agonist, and/or an anticholinergic, and/or a PDE-4 inhibitor, and/or an antihistamine.

Certain compounds of the invention may show selectivity for PI3Kδ over other PI3-kinases. The invention thus provides, in a further aspect, a combination comprising a compound of formula (I) or a pharmaceutically acceptable salt thereof which is selective for PI3Kδ together with a compound or pharmaceutically acceptable salt thereof which is selective for another PI3-kinase, for example PI3Kγ.

One embodiment of the invention encompasses combinations comprising one or two other therapeutic agents.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates to optimise the activity and/or stability and/or physical characteristics, such as solubility, of the therapeutic ingredient. It will be clear also that, where appropriate, the therapeutic ingredients may be used in optically pure form.

In one embodiment, the invention encompasses a combination comprising a compound of formula (I) or a pharmaceutically acceptable salt thereof together with a β₂-adrenoreceptor agonist.

Examples of β₂-adrenoreceptor agonists include salmeterol (which may be a racemate or a single enantiomer such as the R-enantiomer), salbutamol (which may be a racemate or a single enantiomer such as the R-enantiomer), formoterol (which may be a racemate or a single duastereomer such as the R,R-diastereomer), salmefamol, fenoterol carmoterol, etanterol, naminterol, clenbuterol, pirbuterol, flerbuterol, reproterol, bambuterol, indacaterol, terbutaline and salts thereof, for example the xinafoate (1-hydroxy-2-naphthalenecarboxylate) salt of salmeterol, the sulphate salt or free base of salbutamol or the fumarate salt of formoterol. In one embodiment, long-acting β₂-adrenoreceptor agonists, for example, compounds which provide effective bronchodilation for about 12 hrs or longer, are preferred.

Other β₂-adrenoreceptor agonists include those described in WO 02/066422, WO 02/070490, WO 02/076933, WO 03/024439, WO 03/072539, WO 03/091204, WO 04/016578, WO 2004/022547, WO 2004/037807, WO 2004/037773, WO 2004/037768, WO 2004/039762, WO 2004/039766, WO01/42193 and WO03/042160.

Examples of β₂-adrenoreceptor agonists include:

3-(4-{[6-({(2R)-2-hydroxy-2-[4-hydroxy-3-(hydroxymethyl)phenyl]ethyl}amino)hexyl]oxy}butyl)benzenesulfonamide;

3-(3-{[7-({(2R)-2-hydroxy-2-[4-hydroxy-3-hydroxymethyl)phenyl]ethyl}-amino)heptyl]oxy}propyl)benzenesulfonamide;

4-{(1R)-2-[(6-{2-[(2, 6-dichlorobenzyl)oxy]ethoxy}hexyl)amino]-1-hydroxyethyl}-2-(hydroxymethyl)phenol;

4-{(1R)-2-[(6-{4-[3-(cyclopentylsulfonyl)phenyl]butoxy}hexyl)amino]-1-hydroxyethyl}-2-(hydroxymethyl)phenol;

N-[2-hydroxyl-5-[(1R)-1-hydroxy-2-[[2-4-[[(2R)-2-hydroxy-2-phenylethyl]amino]phenyl]ethyl]amino]ethyl]phenyl]formamide;

N-2{2-[4-(3-phenyl-4-methoxyphenyl)aminophenyl]ethyl}-2-hydroxy-2-(8-hydroxy-2(1H)-quinolinon-5-yl)ethylamine; and

5-[(R)-2-(2-{4-[4-(2-amino-2-methyl-propoxy)-phenylamino]-phenyl}-ethylamino)-1-hydroxy-ethyl]-8-hydroxy-1H-quinolin-2-one.

The β₂-adrenoreceptor agonist may be in the form of a salt formed with a pharmaceutically acceptable acid selected from sulphuric, hydrochloric, fumaric, hydroxynaphthoic (for example 1- or 3-hydroxy-2-naphthoic), cinnamic, substituted cinnamic, triphenylacetic, sulphamic, sulphanilic, naphthaleneacrylic, benzoic, 4-methoxybenzoic, 2- or 4-hydroxybenzoic, 4-chlorobenzoic and 4-phenylbenzoic acid.

Suitable anti-inflammatory agents include corticosteroids. Suitable corticosteroids which may be used in combination with the compounds of formula (I) or pharmaceutically acceptable salts thereof are those oral and inhaled corticosteroids and their pro-drugs which have anti-inflammatory activity. Examples include methyl prednisolone, prednisolone, dexamethasone, fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate), 6α,9α-difluoro-11β-hydroxy-16α-methyl-3-oxo-17α-propionyloxy-androsta-1,4-diene-17β-carbothioic acid S-(2-oxo-tetrahydro-furan-3S-yl)ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester and 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester, beclomethasone esters (for example the 17-propionate ester or the 17,21-dipropionate ester), budesonide, flunisolide, mometasone esters (for example mometasone furoate), triamcinolone acetonide, rofleponide, ciclesonide (16α,17-[[(R)-cyclohexylmethylene]bis(oxy)]-11β,21-dihydroxy-pregna-1,4-diene-3,20-dione), butixocort propionate, RPR-106541, and ST-126. Preferred corticosteroids include fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester and 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester. In one embodiment the corticosteroid is 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

Examples of corticosteroids may include those described in WO2002/088167, WO2002/100879, WO2002/12265, WO2002/12266, WO2005/005451, WO2005/005452, WO2006/072599 and WO2006/072600.

Non-steroidal compounds having glucocorticoid agonism that may possess selectivity for transrepression over transactivation and that may be useful in combination therapy include those covered in the following patents: WO03/082827, WO98/54159, WO04/005229, WO04/009017, WO04/018429, WO03/104195, WO03/082787, WO03/082280, WO03/059899, WO03/101932, WO02/02565, WO01/16128, WO00/66590, WO03/086294, WO04/026248, WO03/061651 and WO03/08277. Further non-steroidal compounds are covered in: WO2006/000401, WO2006/000398 and WO2006/015870.

Examples of anti-inflammatory agents include non-steroidal anti-inflammatory drugs (NSAID's).

Examples of NSAID's include sodium cromoglycate, nedocromil sodium, phosphodiesterase (PDE) inhibitors (for example, theophylline, PDE4 inhibitors or mixed PDE3/PDE4 inhibitors), leukotriene antagonists, inhibitors of leukotriene synthesis (for example montelukast), iNOS inhibitors, tryptase and elastase inhibitors, beta-2 integrin antagonists and adenosine receptor agonists or antagonists (e.g. adenosine 2a agonists), cytokine antagonists (for example chemokine antagonists, such as a CCR3 antagonist) or inhibitors of cytokine synthesis, or 5-lipoxygenase inhibitors. An iNOS (inducible nitric oxide synthase inhibitor) is preferably for oral administration. Examples of iNOS inhibitors include those disclosed in WO93/13055, WO98/30537, W002/50021, WO95/34534 and WO99/62875. Examples of CCR3 inhibitors include those disclosed in WO02/26722.

In one embodiment, the invention provides the use of the compounds of formula (I) in combination with a phosphodiesterase 4 (PDE4) inhibitor, especially in the case of a formulation adapted for inhalation. The PDE4-specific inhibitor useful in this aspect of the invention may be any compound that is known to inhibit the PDE4 enzyme or which is discovered to act as a PDE4 inhibitor, and which are only PDE4 inhibitors, not compounds which inhibit other members of the PDE family, such as PDE3 and PDE5, as well as PDE4.

Compounds include cis-4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]. Also, cis-4-cyano-4-[3-(cyclopentyloxy)-4-methoxyphenyl]cyclohexane-1-carboxylic acid (also known as cilomilast) and its salts, esters, pro-drugs or physical forms, which is described in U.S. Pat. No. 5,552,438 issued 3 Sep., 1996; this patent and the compounds it discloses are incorporated herein in full by reference.

Other compounds include AWD-12-281 from Elbion (Hofgen, N. et al. 15th EFMC Int Symp Med Chem (September 6-10, Edinburgh) 1998, Abst P. 98; CAS reference No. 247584020-9); a 9-benzyladenine derivative nominated NCS-613 (INSERM); D-4418 from Chiroscience and Schering-Plough; a benzodiazepine PDE4 inhibitor identified as Cl-1018 (PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Cong Eur Resp Soc (September 19-23, Geneva) 1998] 1998, 12 (Suppl. 28): Abst P2393); roflumilast (CAS reference No 162401-32-3) and a pthalazinone (WO99/47505, the disclosure of which is hereby incorporated by reference) from Byk-Gulden; Pumafentrine, (−)-p-R4aR*,10bS*)-9-ethoxy-1,2,3,4,4a,10b-hexahydro-8-methoxy-2-methylbenzo[c][1,6]naphthyridin-6-yl]-N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al. J Pharmacol Exp Ther, 1998, 284(1): 162), and T2585.

Further compounds are disclosed in the published international patent application WO04/024728 (Glaxo Group Ltd), WO04/056823 (Glaxo Group Ltd) and WO04/103998 (Glaxo Group Ltd) (e.g. Example 399 or 544 disclosed therein). Further compounds are also disclosed in WO2005/058892, WO2005/090348, WO2005/090353, and WO2005/090354, all in the name of Glaxo Group Limited.

Examples of anticholinergic agents are those compounds that act as antagonists at the muscarinic receptors, in particular those compounds which are antagonists of the M₁ or M₃ receptors, dual antagonists of the M₁/M₃ or M₂/M₃, receptors or pan-antagonists of the M₁/M₂/M₃ receptors. Exemplary compounds for administration via inhalation include ipratropium (for example, as the bromide, CAS 22254-24-6, sold under the name Atrovent), oxitropium (for example, as the bromide, CAS 30286-75-0) and tiotropium (for example, as the bromide, CAS 136310-93-5, sold under the name Spiriva). Also of interest are revatropate (for example, as the hydrobromide, CAS 262586-79-8) and LAS-34273 which is disclosed in WO01/04118. Exemplary compounds for oral administration include pirenzepine (CAS 28797-61-7), darifenacin (CAS 133099-04-4, or CAS 133099-07-7 for the hydrobromide sold under the name Enablex), oxybutynin (CAS 5633-20-5, sold under the name Ditropan), terodiline (CAS 15793-40-5), tolterodine (CAS 124937-51-5, or CAS 124937-52-6 for the tartrate, sold under the name Detrol), otilonium (for example, as the bromide, CAS 26095-59-0, sold under the name Spasmomen), trospium chloride (CAS 10405-02-4) and solifenacin (CAS 242478-37-1, or CAS 242478-38-2 for the succinate also known as YM-905 and sold under the name Vesicare).

Additional compounds are disclosed in WO 2005/037280, WO 2005/046586 and WO 2005/104745, incorporated herein by reference. The present combinations include, but are not limited to:

(3-endo)-3-(2,2-di-2-thienylethenyl)-8,8-dimethyl-8-azoniabicyclo[3.2.1]octane iodide;

(3-endo)-3-(2-cyano-2,2-diphenylethyl)-8,8-dimethyl-8-azoniabicyclo[3.2.1]octane bromide;

4-[hydroxy(diphenyl)methyl]-1-{2-[(phenylmethyl)oxy]ethyl}-1-azoniabicyclo[2.2.2]octane bromide; and

(1R,5S)-3-(2-cyano-2,2-diphenylethyl)-8-methyl-8-{2-[(phenylmethyl)oxy]ethyl}-8-azoniabicyclo[3.2.1]octane bromide.

Other anticholinergic agents include compounds which are disclosed in US patent application 60/487981 including, for example:

(3-endo)-3-(2,2-di-2-thienylethenyl)-8,8-dimethyl-8-azoniabicyclo[3.2.1]octane bromide;

(3-endo)-3-(2,2-diphenylethenyl)-8,8-dimethyl-8-azoniabicyclo[3.2.1]octane bromide;

(3-endo)-3-(2,2-diphenylethenyl)-8,8-dimethyl-8-azoniabicyclo[3.2.1]octane 4-methylbenzenesulfonate;

(3-endo)-8,8-dimethyl-3-[2-phenyl-2-(2-thienypethenyl]-8-azoniabicyclo[3.2.1]octane bromide; and/or

(3-endo)-8,8-dimethyl-3-[2-phenyl-2-(2-pyridinyl)ethenyl]-8-azoniabicyclo[3.2.1]octane bromide.

Further anticholinergic agents include compounds which are disclosed in US patent application 60/511009 including, for example:

(endo)-3-(2-methoxy-2,2-di-thiophen-2-yl-ethyl)-8,8-dimethyl-8-azonia-bicyclo[3.2.1]octane iodide;

3-((endo)-8-methyl-8-aza-bicyclo[3.2.1]oct-3-yl)-2,2-diphenyl-propionitrile;

(endo)-8-methyl-3-(2,2,2-triphenyl-ethyl)-8-aza-bicyclo[3.2.1]octane;

3-((endo)-8-methyl-8-aza-bicyclo[3.2.1]oct-3-yl)-2,2-diphenyl-propionamide;

3-((endo)-8-methyl-8-aza-bicyclo[3.2.1]oct-3-yl)-2,2-diphenyl-propionic acid;

(endo)-3-(2-cyano-2,2-diphenyl-ethyl)-8,8-dimethyl-8-azonia-bicyclo[3.2.1]octane iodide;

(endo)-3-(2-cyano-2,2-diphenyl-ethyl)-8,8-dimethyl-8-azonia-bicyclo[3.2.1]octane bromide;

3-((endo)-8-methyl-8-aza-bicyclo[3.2.1]oct-3-yl)-2,2-diphenyl-propan-1-ol;

N-benzyl-3-((endo)-8-methyl-8-aza-bicyclo[3.2.1]oct-3-yl)-2,2-diphenyl-propionamide;

(endo)-3-(2-carbamoyl-2,2-diphenyl-ethyl)-8,8-dimethyl-8-azonia-bicyclo[3.2.1]octane iodide;

1-benzyl-3-[3-((endo)-8-methyl-8-aza-bicyclo[3.2.1]oct-3-yl)-2,2-diphenyl-propyl]-urea;

1-ethyl-3-[3-((endo)-8-methyl-8-aza-bicyclo[3.2.1]oct-3-yl)-2,2-diphenyl-propyl]-urea;

N-[3-((endo)-8-methyl-8-aza-bicyclo[3.2.1]oct-3-yl)-2,2-diphenyl-propyl]-acetamide;

N-[3-((endo)-8-methyl-8-aza-bicyclo[3.2.1]oct-3-yl)-2,2-diphenyl-propyl]-benzamide;

3-((endo)-8-methyl-8-aza-bicyclo[3.2.1]oct-3-yl)-2,2-di-thiophen-2-yl-propionitrile;

(endo)-3-(2-cyano-2,2-di-thiophen-2-yl-ethyl)-8,8-dimethyl-8-azonia-bicyclo[3.2.1]octane iodide;

N-[3-((endo)-8-methyl-8-aza-bicyclo[3.2.1]oct-3-yl)-2,2-diphenyl-propyl]-benzenesulfonamide;

[3-((endo)-8-methyl-8-aza-bicyclo[3.2.1]oct-3-yl)-2,2-diphenyl-propyl]-urea;

N-[3-((endo)-8-methyl-8-aza-bicyclo[3.2.1]oct-3-yl)-2,2-diphenyl-propyl]-methanesulfonamide; and/or

(endo)-3-{2,2-diphenyl-3-[(1-phenyl-methanoyl)-amino]-propyl}-8,8-dimethyl-8-azonia-bicyclo[3.2.1]octane bromide.

Further compounds include:

(endo)-3-(2-methoxy-2,2-di-thiophen-2-yl-ethyl)-8,8-dimethyl-8-azonia-bicyclo[3.2.1]octane iodide;

(endo)-3-(2-cyano-2,2-diphenyl-ethyl)-8,8-dimethyl-8-azonia-bicyclo[3.2.1]octane iodide;

(endo)-3-(2-cyano-2,2-diphenyl-ethyl)-8,8-dimethyl-8-azonia-bicyclo[3.2.1]octane bromide;

(endo)-3-(2-carbamoyl-2,2-diphenyl-ethyl)-8,8-dimethyl-8-azonia-bicyclo[3.2.1]octane iodide;

(endo)-3-(2-cyano-2,2-di-thiophen-2-yl-ethyl)-8,8-dimethyl-8-azonia-bicyclo[3.2.1]octane iodide; and/or

(endo)-3-{2,2-diphenyl-3-[(1-phenyl-methanoyl)-amino]-propyl}-8,8-dimethyl-8-azonia-bicyclo[3.2.1]octane bromide.

In one embodiment the invention provides a combination comprising a compound of formula (I) or a pharmaceutically acceptable salt thereof together with an H1 antagonist.

Examples of H1 antagonists include, without limitation, amelexanox, astemizole, azatadine, azelastine, acrivastine, brompheniramine, cetirizine, levocetirizine, efletirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, olopatadine, picumast, pyrilamine, promethazine, terfenadine, tripelennamine, temelastine, trimeprazine and triprolidine, particularly cetirizine, levocetirizine, efletirizine and fexofenadine. In a further embodiment the invention provides a combination comprising a compound of formula (I) or a pharmaceutically acceptable salt thereof together with an H3 antagonist (and/or inverse agonist). Examples of H3 antagonists include, for example, those compounds disclosed in WO2004/035556 and in WO2006/045416. Other histamine receptor antagonists which may be used in combination with the compounds of the present invention include antagonists (and/or inverse agonists) of the H4 receptor, for example, the compounds disclosed in Jablonowski et al., J. Med. Chem. 46:3957-3960 (2003).

The invention thus provides, in a further aspect, a combination comprising a compound of formula (I) or a pharmaceutically acceptable salt thereof together with a PDE4 inhibitor.

The invention thus provides, in a further aspect, a combination comprising a compound of formula (I) or a pharmaceutically acceptable salt thereof together with a β₂-adrenoreceptor agonist.

The invention thus provides, in a further aspect, a combination comprising a compound of formula (I) or a pharmaceutically acceptable salt thereof together with a corticosteroid.

The invention thus provides, in a further aspect, a combination comprising a compound of formula (I) or a pharmaceutically acceptable salt thereof together with a non-steroidal GR agonist.

The invention thus provides, in a further aspect, a combination comprising a compound of formula (I) or a pharmaceutically acceptable salt thereof together with an anticholinergic.

The invention thus provides, in a further aspect, a combination comprising a compound of formula (I) or a pharmaceutically acceptable salt thereof together with an antihistamine.

The invention thus provides, in a further aspect, a combination comprising a compound of formula (I) or a pharmaceutically acceptable salt thereof together with a PDE4 inhibitor and a β₂-adrenoreceptor agonist.

The invention thus provides, in a further aspect, a combination comprising a compound of formula (I) or a pharmaceutically acceptable salt thereof together with an anticholinergic and a PDE-4 inhibitor.

In a preferred aspect, the invention provides a combination comprising a compound of formula (I) or a pharmaceutically acceptable salt thereof together with a corticosteroid.

In a further preferred aspect, the invention provides a combination comprising a compound of formula (I) or a pharmaceutically acceptable salt thereof together with a β₂-adrenoreceptor agonist.

The combinations referred to above may conveniently be presented for use in the form of a pharmaceutical composition and thus pharmaceutical compositions comprising a combination as defined above together with a pharmaceutically acceptable diluent or carrier represent a further aspect of the invention.

The individual compounds of such combinations may be administered either sequentially or simultaneously in separate or combined pharmaceutical formulations. In one embodiment, the individual compounds will be administered simultaneously in a combined pharmaceutical formulation. Appropriate doses of known therapeutic agents will readily be appreciated by those skilled in the art.

The invention thus provides, in a further aspect, a pharmaceutical composition comprising a combination of a compound of formula (I) or a pharmaceutically acceptable salt thereof together with another therapeutically active agent.

The invention thus provides, in a further aspect, a pharmaceutical composition comprising a combination of a compound of formula (I) or a pharmaceutically acceptable salt thereof together with a PDE4 inhibitor.

The invention thus provides, in a further aspect, a pharmaceutical composition comprising a combination of a compound of formula (I) or a pharmaceutically acceptable salt thereof together with a β₂-adrenoreceptor agonist.

The invention thus provides, in a further aspect, a pharmaceutical composition comprising a combination of a compound of formula (I) or a pharmaceutically acceptable salt thereof together with a corticosteroid.

The invention thus provides, in a further aspect, a pharmaceutical composition comprising a combination of a compound of formula (I) or a pharmaceutically acceptable salt thereof together with a non-steroidal GR agonist.

The invention thus provides, in a further aspect, a pharmaceutical composition comprising a combination of a compound of formula (I) or a pharmaceutically acceptable salt thereof together with an anticholinergic.

The invention thus provides, in a further aspect, a pharmaceutical composition comprising a combination of a compound of formula (I) or a pharmaceutically acceptable salt thereof together with an antihistamine.

The invention thus provides, in a further aspect, a pharmaceutical composition comprising a combination of a compound of formula (I) or a pharmaceutically acceptable salt thereof together with a PDE4 inhibitor and a β₂-adrenoreceptor agonist.

The invention thus provides, in a further aspect, a pharmaceutical composition comprising a combination of a compound of formula (I) or a pharmaceutically acceptable salt thereof together with an anticholinergic and a PDE4 inhibitor.

In a preferred aspect, the invention provides a pharmaceutical composition comprising a combination of a compound of formula (I) or a pharmaceutically acceptable salt thereof together with a corticosteroid.

In a further preferred aspect, the invention provides a pharmaceutical composition comprising a combination of a compound of formula (I) or a pharmaceutically acceptable salt thereof together with a β₂-adrenoreceptor agonist.

The invention will now be illustrated by way of the following non-limiting examples.

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

General Methods

LCMS methods

Method A

The HPLC analysis was conducted on a Sunfire C18 column (30 mm×4.6 mm i.d. 3.5 μm packing diameter) at 30 degrees centigrade.

Solvent A=0.1% v/v solution of Formic Acid in Water.

Solvent B=0.1% v/v solution of Formic Acid in Acetonitrile.

The gradient employed was:

| Time (min) | Flow Rate (ml/min) | % A | % B |
|---|---|---|---|
| 0 | 3 | 97 | 3 |
| 0.1 | 3 | 97 | 3 |
| 4.2 | 3 | 0 | 100 |
| 4.8 | 3 | 0 | 100 |
| 4.9 | 3 | 97 | 3 |
| 5.0 | 3 | 97 | 3 |

The UV detection was an averaged signal from wavelength of 210 nm to 350 nm and mass spectra were recorded on a mass spectrometer using alternate-scan positive and negative mode electrospray ionization.

Method B

The HPLC analysis was conducted on a Acquity UPLC BEH C18 column (50 mm×2.1 mm i.d. 1.7 μm packing diameter) at 40 degrees centigrade.

Solvent A=0.1% v/v solution of Formic Acid in Water.

Solvent B=0.1% v/v solution of Formic Acid in Acetonitrile.

The gradient employed was:

| Time (min) | Flow Rate (ml/min) | % A | % B |
|---|---|---|---|
| 0 | 1 | 97 | 3 |
| 1.5 | 1 | 0 | 100 |
| 1.9 | 1 | 0 | 100 |
| 2.0 | 1 | 97 | 3 |

The UV detection was an averaged signal from wavelength of 210 nm to 350 nm and mass spectra were recorded on a mass spectrometer using alternate-scan positive and negative mode electrospray ionization.

Method C

Waters ZQ mass spectrometer operating in positive ion electrospray mode, mass range 100-1000 amu.

UV wavelength: 215-330 nm

Column: 3.3 cm×4.6 mm i.d., 3 μm ABZ+PLUS

Flow Rate: 3 ml/min

Injection Volume: 5 μl

Solvent A: 95% MeCN+0.05% of a 1% v/v solution of formic acid in water

Solvent B: 0.1% v/v solution of formic acid in 10 mmol ammonium acetate (aq)

Gradient: Mixtures of Solvent A and Solvent B are used according to the following gradient profiles (expressed as % Solvent A in the mixture): 0% A; 0.7 mins, 0-100% A; 3.5 mins, 100% A; 0.4 mins, 100-0% A; 0.2 mins.

MDAP Methods

Mass Directed Automated Preparative HPLC and MS Conditions

Method A

Stationary Phase

The stationary phase used for this purification was Sunfire C18 with a particle size of 5 μm.

Small Scale Preparative Column

Column Dimension: 100 mm×19 mm i.d.

Large Scale Preparative Column

Column Dimension: 150 mm×30 mm i.d.

Eluent

The eluents employed were:

A=0.1% v/v solution of formic acid in water.

B=0.1% v/v solution of formic acid in acetonitrile.

Methods for Small Scale Prep for Up to 30 mg of Crude Sample

There are ten focused small scale preparative methods available for use. The choice of method is dependent on two factors
1. The retention time (RT) of the component/s of interest on the generic analytical LCMS method.
2. The presence of closely eluting impurities to the component/s of interest.

From the analytical RT the choice of one of five small scale focused prep methods is made. Small scale prep methods contain a 10 minute gradient over a specified organic range, followed by a 5 minute flush, except the most polar method which contains a 7 minute gradient over a specified organic range followed by an 8 minute flush. The total run time is 15 minutes.

If there are closely eluting impurities to the component/s of interest then there are five extended small scale focused prep methods available. Extended small scale prep methods contain a 20 minute gradient over the specified organic range followed by a 5 minute flush, except the most polar method which contains a 14 minute gradient over the specified organic range followed by an 11 minute flush. The total run time is 25 minutes.

Flow rates for all small scale methods are 20 ml/min and the purification is performed at ambient temperature.

The injection volume for small scale prep is 500 μl.

The 10 small scale prep methods and the organic ranges of the gradients are shown below. The gradients are the same for normal or extended runs.

5-30% B

15-55% B

30-85% B

50-99% B

80-99% B

In the flush step eluent B is raised to 99% in 0.5 minutes then held there for a further 4.5 minutes.

Methods for Large Scale Prep for Up to 90 mg of Crude Sample

There are ten focused large scale prep methods available for use. The choice of method is dependent on the same two factors as for small scale prep. The run times (gradient and flush) are the same as for small scale prep methods.

Flow rates for all large scale methods are 40 ml/min and the purification is performed at ambient temperature.

The injection volume for large scale prep is 980 μl.

The 5 large scale method names and the organic ranges of the gradients are shown below. The gradients are the same for either normal or extended runs.

5-30% B

15-55% B

30-85% B

50-99% B

80-99% B

In the flush step eluent B is raised to 99% in 0.5 minutes then held there for a further 4.5 minutes.

UV Detection

The UV detection for all methods is an averaged signal from all wavelengths from 210 nm to 350 nm.

MS Conditions

MS: Waters ZQ

Ionisation mode: Alternate-scan positive and negative electrospray

Scan range: 100 to 1000 amu

Scan time: 0.50 seconds

Inter scan delay: 0.20 seconds

Method B

Column Details: XBRIDGE C₁₈ column (100 mm×19 mm i.d., 5 μm packing diameter)

Solvents:

A: 10 mmol ammonium bicarbonate (aq) adjusted to pH 10 with ammonia (aq)

B: MeCN

The UV detection was an averaged signal from wavelength of 210 nm to 350 nm and mass spectra were recorded on a mass spectrometer using an alternate-scan positive and negative mode electrospray ionization.

Method C

Sunfire, Low pH

Column Details: SUNFIRE C18 column (100 mm×19 mm id. 5 um)

The solvents employed were:

A=0.1% v/v solution of Formic Acid in Water.

B=0.1% v/v solution of Formic Acid in Acetontrile.

Method A

| Time (min) | Flow Rate (ml/min) | % A | % B |
|---|---|---|---|
| 0.0 | 20 | 95 | 5 |
| 1.0 | 20 | 95 | 5 |
| 10 | 20 | 70 | 30 |
| 10.5 | 20 | 5 | 95 |
| 12.5 | 20 | 5 | 95 |
| 13 | 20 | 95 | 5 |
| 14 | 20 | 95 | 5 |

Collection was triggered by uv, ms or a combination of the two.

The UV detection was at a selected wavelength generally 230 nm, 210 nm or 254 nm. Mass spectra were recorded on a mass spectrometer using an alternate-scan positive and negative mode electrospray ionization.

Method B

| Time (min) | Flow Rate (ml/min) | % A | % B |
|---|---|---|---|
| 0.0 | 20 | 85 | 15 |
| 1.0 | 20 | 85 | 15 |
| 10 | 20 | 45 | 55 |
| 10.5 | 20 | 5 | 95 |
| 12.5 | 20 | 5 | 95 |
| 13 | 20 | 85 | 15 |
| 14 | 20 | 85 | 15 |

Collection was triggered by uv, ms or a combination of the two.

The UV detection was at a selected wavelength generally 230 nm, 210 nm or 254 nm. Mass spectra were recorded on a mass spectrometer using an alternate-scan positive and negative mode electrospray ionization.

Method C

| Time (min) | Flow Rate (ml/min) | % A | % B |
|---|---|---|---|
| 0.0 | 20 | 70 | 30 |
| 1.0 | 70 | 70 | 30 |
| 10 | 20 | 15 | 85 |
| 10.5 | 20 | 5 | 95 |
| 12.5 | 20 | 5 | 95 |
| 13 | 20 | 70 | 30 |
| 14 | 20 | 70 | 30 |

Collection was triggered by uv, ms or a combination of the two.

The UV detection was at a selected wavelength generally 230 nm, 210 nm or 254 nm. Mass spectra were recorded on a mass spectrometer using an alternate-scan positive and negative mode electrospray ionization.

Method D

| Time (min) | Flow Rate (ml/min) | % A | % B |
|---|---|---|---|
| 0.0 | 20 | 50 | 50 |
| 1.0 | 20 | 50 | 50 |
| 10 | 20 | 1 | 99 |
| 12.5 | 20 | 1 | 99 |
| 13 | 20 | 50 | 50 |
| 14 | 20 | 50 | 50 |

Collection was triggered by uv, ms or a combination of the two.

The UV detection was at a selected wavelength generally 230 nm, 210 nm or 254 nm. Mass spectra were recorded on a mass spectrometer using an alternate-scan positive and negative mode electrospray ionization.

Method E

| Time (min) | Flow Rate (ml/min) | % A | % B |
|---|---|---|---|
| 0.0 | 20 | 20 | 80 |
| 1.0 | 20 | 20 | 80 |
| 7.0 | 20 | 1 | 99 |
| 12.5 | 20 | 1 | 99 |
| 13 | 20 | 20 | 80 |
| 14 | 20 | 20 | 80 |

Collection was triggered by uv, ms or a combination of the two.

The UV detection was at a selected wavelength generally 230 nm, 210 nm or 254 nm. Mass spectra were recorded on a mass spectrometer using an alternate-scan positive and negative mode electrospray ionization.

### INTERMEDIATES AND EXAMPLES

When the name of a commercial supplier is given after the name of a compound or a reagent, for instance “compound X (Aldrich)” or “compound X/Aldrich”, this means that compound X is obtainable from a commercial supplier, such as the commercial supplier named. If not referenced herein the compound or reagent can be purchased from a standard supplier such as Sigma Aldrich, Lancaster, Fluorochem, TCI etc.

Similarly, when a literature or a patent reference is given after the name of a compound, for instance compound Y (EP 0 123 456), this means that the preparation of the compound is described in the named reference.

The names of the Examples have been obtained using a compound naming programme which matches name to structure (e.g. ACD/Name Batch v 9.0).

### Intermediate 1

### 1-(Phenylsulfonyl)-6-(trimethylstannanyl)-1H-indazol-4-amine

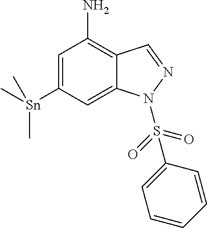

A mixture of 6-bromo-1-(phenylsulfonyl)-1H-indazol-4-amine (1.3 g), hexamethylditin (2.4 g), triethylamine (1 ml) and Pd(PPh₃)₄ (0.2 g) in toluene (15 ml) was heated under microwave irradiation at 120° C. for 1 h. The reaction was applied to a silica cartridge using light petroleum 40-60° C. as eluent. This was changed to ether/light petroleum 40-60° C. The appropriate fractions were evaporated to give the title compound, 1.2 g.

LCMS (method A); Rₜ=3.3 min, MH⁺=438.

### Intermediate 2

### 6-Bromo-1-(phenylsulfonyl)-1H-indazol-4-amine

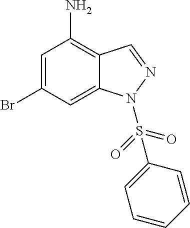

6-Bromo-1H-indazol-4-amine (5 g) was dissolved in DMF (20 ml) and cooled in an ice bath. 60% Sodium hydride in mineral oil (0.94 g) was added portionwise and the reaction was left under an ice bath for 30 min. Benzenesulfonyl chloride (3 ml) in DMF (5 ml) was added slowly over 15 min and the reaction was left to warm up to RT overnight. Water (100 ml) was added and the reaction stirred for 20 min. Ethyl acetate (120 ml) was added and the water was separated, washed with ethyl acetate (50 ml×2) and the combined organics were washed with 7.5% lithium chloride (aq) (50 ml×2) then water (50 ml) before being separated and passed through a hydrophobic frit. The ethyl acetate was evaporated and the residue passed through a silica cartridge, eluting with DCM (ca. 300 ml) followed by diethyl ether (ca. 400 ml). Product containing pure fractions were combined and evaporated to dryness to give the title compound, 5.9 g.

LCMS (method B); Rₜ=1.12 min, MH⁺=354.

### Intermediate 3

### 2-(Chloromethyl)-1,3-thiazole-4-carbonyl chloride

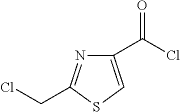

To a solution of 2-(hydroxymethyl)-1,3-thiazole-4-carboxylic acid (370 mg) in chloroform (5 ml) and DMF (0.1 ml) was added thionyl chloride (1 ml). The mixture was heated to reflux for 1 h. The mixture was cooled and the solvent removed in vacuo. The residue was azeotroped with chloroform (5 ml) and dried on a high vacuum line for 30 min to afford the title compound. The material was not suitable for long term storage at RT so was either used immediately or stored below RT for up to 2 weeks.

LCMS was run as a solution in MeOH (method B); Rₜ=0.77 min, MH⁺=191.

### Intermediate 4

### 2-(Hydroxymethyl)-1,3-thiazole-4-carboxylic acid

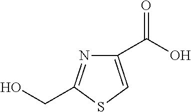

A solution of 2-{[(2,2-dimethylpropanoyl)oxy]methyl}-1,3-thiazole-4-carboxylic acid (3 g) and potassium carbonate (2.33 g) in methanol (100 ml) and water (30 ml) was heated to reflux for 4 h. The mixture was cooled and concentrated in vacuo to ˜30 ml. It was then acidified with 2 M HCl (aq) (50 ml) and concentrated in vacuo. The resulting solid was treated with hot MeOH/EtOAc (2:1), washing well before filtering off the remaining solid. The filtrate was concentrated in vacuo to give a brown solid which was dissolved in MeOH and added to the top of 2×70 g aminopropyl cartridge that had been preconditioned with MeOH. The cartridges were both eluted with MeOH and then with 10% HCl in MeOH. The acidic fractions were combined and the solvent removed in vacuo to give the title compound as a brown oil, 550 mg.

LCMS (method B); Rₜ=0.38 min, MH⁺=160.

### Intermediate 5

### 2-(Chloromethyl)-N-[6-(1H-indol-4-yl)-1-(phenylsulfonyl)-1H-indazol-4-yl]-1,3-thiazole-4-carboxamide

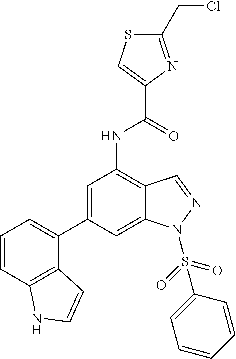

To solution of 6-(1H-indol-4-yl)-1-(phenylsulfonyl)-1H-indazol-4-amine (1.5 g) in chloroform (20 ml) at 0° C. was added DIPEA (1.35 ml). 2-(Chloromethyl)-1,3-thiazole-4-carbonyl chloride (1.8 g) in chloroform (20 ml) was added dropwise and the mixture was stirred at 0° C. for 1 h 15 min. The mixture was allowed to warm to RT and stirring continued for 18 h. A further portion of 2-(chloromethyl)-1,3-thiazole-4-carbonyl chloride (0.2 g) was added to the mixture which was stirred at RT for 30 min. Water (50 ml) was added and the mixture was extracted with DCM (2×100 ml), separating the layers using a hydrophobic frit. The organics were collected and solvent removed in vacuo to give a brown solid which was triturated with ether (10 ml). The solid was filtered and dried in a vacuum oven overnight to afford the title compound, 1.6 g.

LCMS (Method B): Rₜ=1.26 min, MH⁺=548.

### Intermediate 6

### 6-(1H-Indol-4-yl)-1-(phenylsulfonyl)-1H-indazol-4-amine

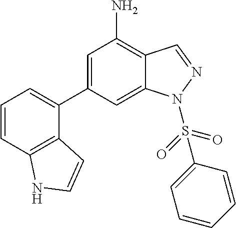

6-Bromo-1-(phenylsulfonyl)-1H-indazol-4-amine (3 g), 4-(4,4,5,5-tetramethyl-1,3,2-dioxaborolan-2-yl)-1H-indole (2.278 g), Pd(dppf)Cl₂ (0.623 g) and sodium carbonate (2.71 g) were divided between 2 microwave vials and dissolved in 1,4-dioxane (16 ml) and water (16 ml) to give 8 ml of each solvent in each vial. The vials were heated in the microwave at 100° C. for 10 min. The mixtures were combined and filtered through Celite, washing with EtOAc. The resulting mixture was partitioned between water (100 ml) and EtOAc (100 ml) and the layers separated. The aqueous layer was extracted with further EtOAc (2×50 ml) and the organic extracts were combined, passed through a hydrophobic frit and the solvent removed in vacuo to give a brown solid which was pre-adsorbed onto silica and added to the top of a 100 g silica SPE cartridge. This was eluted with 0-100% EtOAc:cyclohexane over 60 min on the FlashMaster II. The product-containing fractions were combined and the solvent was removed in vacuo to afford the title compound as orange crystals which were dried on a high vacuum line for 1 h.

LCMS (Method B): Rₜ=1.11 min, MH⁺=388.

### Intermediate 7

### 2-(Chloromethyl)-N-[6-{6-(methyloxy)-5-[(methylsulfonyl)amino]-3-pyridinyl}-1-(phenylsulfonyl)-1H-indazol-4-yl]-1,3-thiazole-4-carboxamide

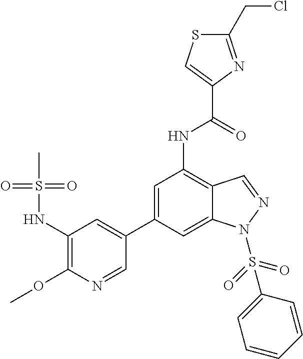

N-[5-[4-Amino-1-(phenylsulfonyl)-1H-indazol-6-yl]-2-(methyloxy)-3-pyridinyl]methanesulfonamide (700 mg) in DCM (30 ml) was cooled to 0° C. before pyridine (0.179 ml) was added. 2-(Chloromethyl)-1,3-thiazole-4-carbonyl chloride (290 mg) in DCM (10 ml) was added dropwise over 15 min. The mixture was stirred at 0° C. for 30 min. Saturated aq. sodium bicarbonate solution (20 ml) was added to the mixture and it was stirred vigorously for 10 min. The organic layer was separated using a hydrophobic frit, washed with water (20 ml) and concentrated in vacuo to give the title compound as an orange solid, 697 mg.

LCMS (Method B); Rₜ=1.14 min, MH⁺=634.

### Intermediate 8

### N-[5-[4-Amino-1-(phenylsulfonyl)-1H-indazol-6-yl]-2-(methyloxy)-3-pyridinyl]methanesulfonamide

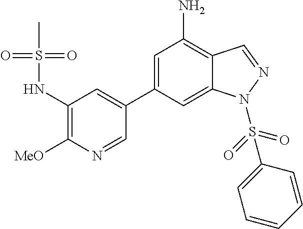

1-(Phenylsulfonyl)-6-(trimethylstannanyl)-1H-indazol-4-amine (4.2 g), N-[5-bromo-2-(methyloxy)-3-pyridinyl]methanesulfonamide (3.25 g) and Pd(PPh₃)₄ (1.113 g) were weighed into a round-bottomed flask. DMF (30 ml) was added and the reaction was heated at 120° C. for 20 min. The solvent was removed in vacuo and the crude material was dissolved in DCM and loaded onto the top of a 750 g RediSep silica cartridge that was then eluted with 0-100% EtOAc:cyclohexane over 10 column volumes using the Companion XL. The product-containing fractions were combined and the solvent removed in vacuo to afford the title compound as a pale yellow solid, 2.39 g.

LCMS (Method B) Rₜ=0.95 min, MH⁺=474.

### Intermediate 9

### N-[5-Bromo-2-(methyloxy)-3-pyridinyl]methanesulfonamide

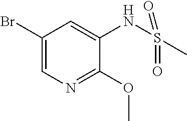

To 5-bromo-2-(methyloxy)-3-pyridinamine (3.11 g) in pyridine (30 ml) was added methanesulfonyl chloride (2.96 ml) and the reaction was stirred for 18 h at RT. After this time, the mixture was adjusted to pH 7 with 2M HCl (aq) and extracted into DCM. The combined organic layers were evaporated to dryness. The residual solid was dissolved in methanol (15 ml) before 2M NaOH (15 ml) was added and the mixture stirred for 30 min at RT. The methanol was removed in vacuo and the aqueous mixture was washed with DCM. The DCM was separated from the aqueous which was then acidified to pH 2 with 2M HCl. The resulting precipitate was collected by vacuum filtration to give the title compound, 1.79 g.

LCMS (Method B) Rₜ=0.86 min, MH⁺=282.

### Intermediate 10

### 6-Bromo-4-nitro-2-(tetrahydro-2H-pyran-2-yl)-2H-indazole

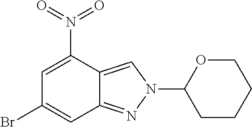

To 6-bromo-4-nitro-1H-indazole (10 g) in dihydropyran (100 ml) was added TFA (0.068 ml) and the reaction was heated for 1.5 h at reflux. After cooling, DCM (180 ml) and saturated sodium bicarbonate solution (50 ml) was added and stirred for 10 min. The DCM was separated from the aq. which was re-washed with DCM (70 ml). The combined organic layers were passed through a hydrophobic frit and evaporated to dryness. The residual solid was triturated with ether then filtered. The solid material was dissolved in DCM and purified by chromatography on silica on the ISCO Companion, using an isocratic gradient of DCM. Purified fractions were combined and evaporated to dryness to afford the title compound, 7.78 g.

LCMS (method C); Rₜ=3.51 min, MH⁻=326/328.

### Intermediate 11

### 6-Bromo-2-(tetrahydro-2H-pyran-2-yl)-2H-indazol-4-amine

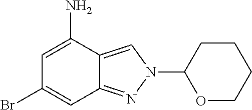

6-Bromo-4-nitro-2-(tetrahydro-2H-pyran-2-yl)-2H-indazole (6 g), iron filings (3.29 g) and ammonium chloride (0.492 g) were weighed to a 250 ml round-bottomed flask and ethanol (60 ml) then water (18 ml) were added. The reaction was heated to 80° C. for 2.5 h. The reaction mixture was cooled. Ethyl acetate (100 ml) and water (50 ml) were added. There was no visible separation of layers so the reaction was concentrated to remove the ethyl acetate and ethanol. Ethyl acetate (250 ml) was added and the organic layer was washed with water (50 ml), before passing through a hydrophobic frit. The organic layer was evaporated to dryness. The residue was purified by column chromatography on silica (120 g silica column, ISCO Companion) eluting with a gradient of 1-2% methanol in DCM over 25 min. Fractions containing desired material were combined and evaporated to dryness to afford the title compound, 3.95 g.

LCMS (method C); Rₜ=2.87 min, MH⁻=298.

### Intermediate 12

### 2-(Methyloxy)-5-(4,4,5,5-tetramethyl-1,3,2-dioxaborolan-2-yl)-3-pyridinamine

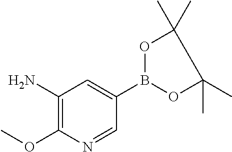

To 5-bromo-2-(methyloxy)-3-pyridinamine (18.93 g) in a round-bottom flask was added nitrogen-purged 1,4-dioxane (500 ml) followed by 4,4,4′,4′,5,5,5′,5′-octamethyl-2,2′-bi-1,3,2-dioxaborolane (47.4 g), potassium acetate (27.5 g) and Pd(dppf)Cl₂—CH₂Cl₂ adduct (7.61 g). The mixture was then stirred at 80° C. under nitrogen for 2 h. The reaction mixture was allowed to cool then partitioned between ethyl acetate and water. The mixture was filtered through a celite pad and the aqueous layer extracted twice with ethyl acetate. The combined organics were washed with water, brine and dried over magnesium sulphate overnight. The residue was purified on 1.5 Kg Silica cartridge, using a 0-50% ethyl acetate:DCM gradient over 10 column volumes. The appropriate fractions were combined and evaporated to dryness. The residue was triturated with cyclohexane, filtered and dried in vacuo to give the title compound as a light pink solid, 11.1 g. A second crop was obtained from the filtrate and after drying gave a further portion of the title compound as a light pink solid, 2.95 g.

LCMS (method B); Rₜ 0.91=min, MH⁺=251.

### Intermediate 13

### N-[2-(Methyloxy)-5-(4,4,5,5-tetramethyl-1,3,2-dioxaborolan-2-yl)-3-pyridinyl]methanesulfonamide

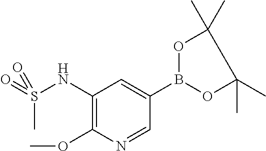

To a solution of 2-(methyloxy)-5-(4,4,5,5-tetramethyl-1,3,2-dioxaborolan-2-yl)-3-pyridinamine (0.5 g) in pyridine (5 ml) was added methanesulphonyl chloride (0.309 ml) and the mixture stirred at RT for 18 h when the solvent was removed in vacuo. The residue was partitioned between saturated sodium bicarbonate solution (10 ml) and DCM (20 ml), separated by hydrophobic frit and purified by silica (70 g) cartridge on Flashmaster II using a gradient of DCM and methanol to give the title compound as a brown solid, 0.46 g.

LCMS (method B); Rₜ=0.98 min, MH⁺=329.

### Intermediate 14

### Ethyl 1-(tetrahydro-2H-pyran-4-ylmethyl)-1H-pyrazole-3-carboxylate and ethyl 1-(tetrahydro-2H-pyran-4-ylmethyl)-1H-pyrazole-5-carboxylate

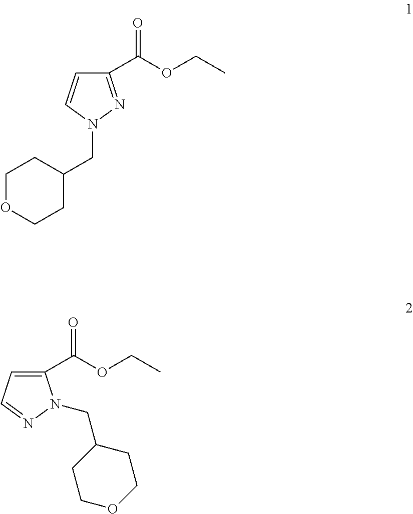

To a solution of ethyl 1H-pyrazole-3-carboxylate (2 g) in MeCN (40 ml) was added cesium carbonate (4.65 g) and the mixture stirred for 30 min when 4-(bromomethyl)tetrahydro-2H-pyran (2.81 g) was added and the mixture stirred at RT for 18 h. The solvent was removed in vacuo and the residue partitioned between dichloromethane (50 ml) and water (20 ml), separated by hydrophobic frit and concentrated. The filtrate was purified by silica (2×100 g) cartridge on Flashmaster II using a gradient of DCM and ethyl acetate to give the title compound 1 as a colourless oil, 0.944 g and title compound 2, as a colourless oil, 1.026 g.

1; LCMS (method B); Rₜ=0.90 min, MH⁺=239.

2; LCMS (method B); Rₜ=0.75 min, MH⁺=239.

### Intermediate 15

### 1-(Tetrahydro-2H-pyran-4-ylmethyl)-1H-pyrazole-3-carboxylic acid

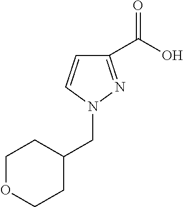

To a solution of ethyl 1-(tetrahydro-2H-pyran-4-ylmethyl)-1H-pyrazole-3-carboxylate (0.5 g) in methanol (15 ml) was added 2 M sodium hydroxide (aq) (10 ml) and the mixture stirred at RT for 18 h. The solvent was removed in vacuo and the residue was taken up in water (5 ml) and acidified to pH 1 using 2 M HCl (aq) then evaporated to dryness. The residue was triturated with ethyl acetate (20 ml), filtered and concentrated to give the title compound as a white solid, 0.43 g.

LCMS (method B); Rₜ=0.51 min, MH⁺=211.

### Intermediate 16

### 1-(Tetrahydro-2H-pyran-4-ylmethyl)-1H-pyrazole-5-carboxylic acid

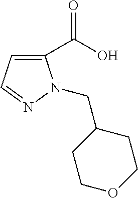

Prepared similarly to Intermediate 15, starting from ethyl 1-(tetrahydro-2H-pyran-4-ylmethyl)-1H-pyrazole-5-carboxylate, to give the title compound, 0.352 g.

LCMS (method A); Rₜ=1.48 min, MH⁺=211.

### Intermediate 17

### N-[6-Bromo-2-(tetrahydro-2H-pyran-2-yl)-2H-indazol-4-yl]-1-(tetrahydro-2H-pyran-4-ylmethyl)-1H-pyrazole-3-carboxamide

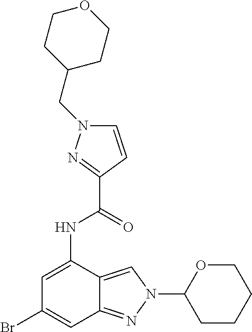

1-(Tetrahydro-2H-pyran-4-ylmethyl)-1H-pyrazole-3-carboxylic acid (100 mg) was dissolved in DCM (3 ml) and cooled to 0° C. before (1-chloro-2-methyl-1-propen-1-yl)dimethylamine (0.126 ml) was added dropwise. The reaction was stirred for 20 min, before 6-bromo-2-(tetrahydro-2H-pyran-2-yl)-2H-indazol-4-amine (127 mg) in DCM (2 ml) and DIPEA (0.166 ml) were added. The reaction was warmed to RT over 2 h. Saturated sodium bicarbonate solution was added and the reaction stirred for 20 min. The DCM was passed through a hydrophobic frit, then evaporated to dryness. The residue was purified on the ISCO companion using a 12 g silica cartridge, and 50-100% ethyl acetate in cyclohexane over 15 min, 25 ml/min. Product containing fractions were combined and evaporated to dryness to give the title compound, 71.1 mg.

LCMS (method B); Rₜ=1.07 min, MH⁺=490.

### Intermediate 18

### 6-(1H-Indol-4-yl)-2-(tetrahydro-2H-pyran-2-yl)-2H-indazol-4-amine

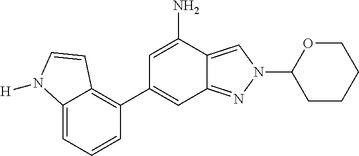

6-(1H-Indol-4-yl)-4-nitro-2-(tetrahydro-2H-pyran-2-yl)-2H-indazole (714 mg) was dissolved in ethyl acetate (100 ml) and the compound was hydrogenated using the H-cube® (available from THALESNano) using a 10% Pd/C catalyst at 30° C. and under 30 bar pressure of hydrogen. The solvent was removed in vacuo to give the title compound, 629 mg.

LCMS (method C); Rₜ=2.86 mins, MH⁺=333.

### Intermediate 19

### 6-(1H-Indol-4-yl)-4-nitro-2-(tetrahydro-2H-pyran-2-yl)-2H-indazole

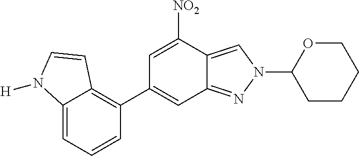

Five reactions were set up with 6-bromo-4-nitro-2-(tetrahydro-2H-pyran-2-yl)-2H-indazole (500 mg), Pd(dppf)Cl₂ (125 mg), 1H-indol-4-ylboronic acid (370 mg), saturated sodium hydrogen carbonate (aq) (3 ml) and IPA (12 ml) in each. They were all heated at 150° C. for 10 min in the microwave. The reaction mixtures were combined and water (250 ml) and ethyl acetate (250 ml) added. The mixture was filtered and the organic layer collected. The organic layer was washed with water followed by brine. The organic layer was dried over magnesium sulphate, filtered and the solvent was removed in vacuo. The residue was dissolved in DCM and pre-absorbed onto silica. Purification was carried out using chromatography on silica eluting with 0-25% ethyl acetate in cyclohexane. The desired fractions were collected and combined and solvent removed in vacuo to give the title compound as a yellow solid, 1.72 g.

LCMS (method C); R_{f}=3.61 min, MH⁺=363.

### Intermediate 20

### 6-(1H-Indol-4-yl)-1H-indazol-4-amine

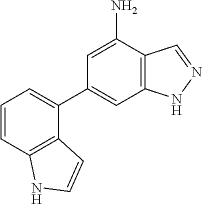

6-Bromo-1H-indazol-4-amine (10 g) (available from Sinova Inc.) and 4-(4,4,5,5-tetramethyl-1,3,2-dioxaborolan-2-yl)-1H-indole (16.05 g) (available from Frontier Scientific, Europe Ltd) were dissolved in 1,4-dioxane (60 ml) and water (60 ml). 2 M sodium carbonate (70.7 ml) and Pd(dppf)Cl₂-DCM adduct (1.93 g) were added and the mixture was heated at 115° C. for 18 h. The reaction mixture was diluted with DCM (200 ml) and the organic and aq layers were separated using a hydrophobic frit. The aq layer was extracted with further quantities of DCM (2×200 ml), using a hydrophobic frit to separate the layers. The organic layers were combined and silica (80 g) was added. The solvent was removed in vacuo to give a crude material that was purified by chromatography on silica gel (750 g cartridge, Flashmaster II) eluting with 0-100% ethyl acetate in cyclohexane over 60 min. The oil was dried in vacuo overnight. The yellow foam was dissolved in DCM (3×400 ml), removing the solvent in vacuo after each dissolution. ethyl acetate (50 ml) was then added and the solvent was removed in vacuo. The solid obtained was dried in a vacuum oven to afford the title compound (12.8 g) as a yellow foam.

LC/MS (method A); Rₜ=2.71 min, MH⁺=249.

### Intermediate 21

### N-{5-[4-Amino-1-(phenylsulfonyl)-1H-indazol-6-yl]-2-chloro-3-pyridinyl}methanesulfonamide

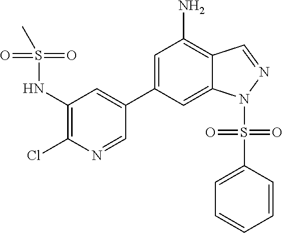

Four identical reactions were carried out in a 20 ml microwave vial, each using one quarter of the quantities stated. The reactions were then combined for work-up and purification. A solution of a quarter of 1-(phenylsulfonyl)-6-(trimethylstannanyl)-1H-indazol-4-amine (4.329 g) in a quarter of DMF (24 ml) was treated with a quarter of N-(5-bromo-2-chloro-3-pyridinyl)methanesulfonamide (3.26 g) followed by a quarter of Pd(PPh₃)₄ (1.032 g). Each vial was heated at 120° C. for 50 min in a Biotage Initiator. The orange solutions were combined and loaded onto a 20 g silica cartridge, and the vials washed with methanol (10 ml) which was also loaded onto the cartridge which was then eluted with methanol. Then eluants were evaporated to leave an orange oil. This was diluted with DCM (10 ml) and loaded directly and evenly onto 2×100 g silica cartridges. These were eluted with 0-100% ethyl acetate/cyclohexane over 80 min using the Flashmaster II. Appropriate pure fractions were combined and evaporated to give the title compound as a yellow solid (0.939 g).

LCMS (Method B); Rₜ=0.94 min, MH⁺=478.

### Intermediate 22

### N-{5-[4-Amino-1-(phenylsulfonyl)-1H-indazol-6-yl]-3-pyridinyl}methanesulfonamide

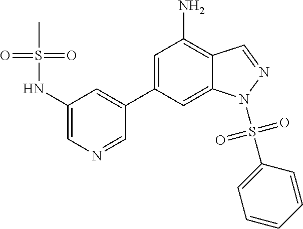

1-(Phenylsulfonyl)-6-(trimethylstannanyl)-1H-indazol-4-amine (700 mg), N-(5-bromo-3-pyridinyl)methanesulfonamide (484 mg) and Solvias catalyst (45 mg) were dissolved in DMF (10 ml) and the reaction was heated under microwave irradiation for 30 min at 120° C. In a separate microwave vial, 1-(phenylsulfonyl)-6-(trimethylstannanyl)-1H-indazol-4-amine (50 mg), N-(5-bromo-3-pyridinyl)methanesulfonamide (35 mg) and Solvias Catalyst (3.2 mg) were dissolved in DMF (0.5 ml) and the reaction was heated under microwave irradiation for 30 minutes at 120° C. The two reactions were combined and poured into water (350 ml) giving a precipitate that was filtered. The filtrate was extracted with ethyl acetate and evaporated to dryness. The precipitate and filtrate residue were separately dissolved in methanol and adsorbed onto fluorosil then purified by column chromatography on silica, eluting with 50-100% ethyl acetate:cyclohexane then 0-5% methanol:ethyl acetate. Combining the product-containing fractions and evaporating to dryness gave the title compound, 314 mg.

LCMS (Method B); Rₜ=0.85 min, MH⁺=444.

### Intermediate 23

### N-{5-[4-Amino-1-(phenylsulfonyl)-1H-indazol-6-yl]-3-pyridinyl}benzenesulfonamide

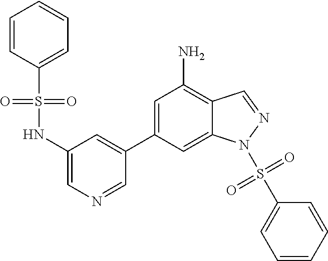

1-(Phenylsulfonyl)-6-(trimethylstannanyl)-1H-indazol-4-amine (500 mg), N-(5-bromo-3-pyridinyl)benzenesulfonamide (395 mg) and Solvias catalyst (64 mg) were dissolved in DMF (10 ml) and heated under microwave irradiation at 120° C. for 15 min. The reaction was cooled to RT then heated under microwave irradiation for another 10 minutes at 120° C. In a separate microwave vial, 1-(phenylsulfonyl)-6-(trimethylstannanyl)-1H-indazol-4-amine (50 mg), N-(5-bromo-3-pyridinyl)benzenesulfonamide (39.5 mg) and Solvias catalyst (6.4 mg) were dissolved in DMF (1 ml) and the reaction was heated under microwave irradiation for 15 min at 120° C. The two reactions were combined and partitioned between EtOAc (100 ml) and water (100 ml) before being evaporated to dryness. The residue was dissolved in DCM:methanol, preadsorbed on fluorosil then purified by column chromatography on silica, eluting with 0-10% methanol in DCM, to give the title compound, 294 mg.

LCMS (Method B); Rₜ=1 min, MH⁺=506.

### Intermediate 24

### 1-(Phenylsulfonyl)-6-(4,4,6,6-tetramethyl-1,3,2-dioxaborinan-2-yl)-1H-indazol-4-amine

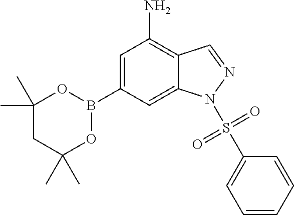

This reaction was carried out in 5 separate microwave vials. Solvent was added per vial totalling 90 ml for the entire reaction. In 5 separate microwave reaction vials was introduced 6-bromo-1-(phenylsulfonyl)-1H-indazol-4-amine (1.078 g×5), potassium acetate (903 mg×5), 4,4,4′,4′,6,6,6′,6′-octamethyl-2,2′-bi-1,3,2-dioxaborinane (1.249 g×5) and Pd(dppf)Cl₂ (224 mg×5). Further to each microwave vial was added 1, 4-dioxane (18 ml×5). The reaction mixtures were then heated at 80° C. for 60 min in the microwave. The reaction mixtures were combined and washed through a silica cartridge (20 g×2, preconditioned with methanol) with methanol. The resulting solution was dried on the rotary evaporator. The residue was extracted with DCM, separated between DCM and water and passed through a hydrophobic frit. The DCM was dried down to afford a solid which was dissolved in DCM, adsorbed onto fluorasil and purified on the ISCO companion using a silica column (120 g). Fractions containing product were combined to give a yellow sticky gum which was refluxed in n-hexane at 80° C. for 2 hours. The solid obtained was dissolved in a minimum amount of methanol, evaporated in vacuo, then dried overnight under vauum to afford the title compound, 4.85 g.

LCMS (Method B); Rₜ=0.96 min, MH⁺=414

### Intermediate 25

### N-{5-[4-Amino-1-(phenylsulfonyl)-1H-indazol-6-yl]-2-chloro-3-pyridinyl}benzenesulfonamide

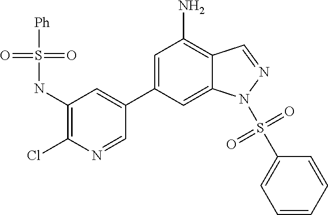

1-(Phenylsulfonyl)-6-(4,4,6,6-tetramethyl-1,3,2-dioxaborinan-2-yl)-1H-indazol-4-amine (300 mg), tripotassium phosphate (462 mgl), Solvias catalyst (81 mg) and N-(5-bromo-2-chloro-3-pyridinyl)benzenesulfonamide (278 mg) were added to a microwave reaction vial. 1,4-Dioxane (3.5 ml) and water (0.7 ml) were added and the reaction was heated for a total of 10 min at 100° C. under microwave conditions. The reaction mixture was washed through a preconditioned (with methanol) silica cartridge (1 g) with methanol. The resulting solution was dried. This redisue was purified using a silica cartridge (5 g×2, preconditioned with DCM) and eluting with DCM. Fractions containing the product were combined and evaporated in vacuo then dried under high vacuum. The residue was dissolved in DCM and evaporated in vacuo then dried under high vacuum to afford the title compound, 392 mg.

LCMS (Method B); Rₜ=1.11 min, MH⁺=541.

### Intermediate 26

### 6-Bromo-3-fluoro-4-nitro-1H-indazole

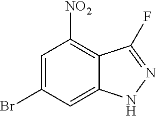

To a solution of 6-bromo-4-nitro-1H-indazole (5 g) in acetonitrile (50 ml) and acetic acid (10 ml) was added Selectfluor (9.39 g). The resulting mixture was heated to 100° C. and stirred for two days. The reaction mixture was concentrated under vacuum. The residue was dissolved in DCM and then filtered off. The sample was absorbed onto silica powder then solid loaded onto the companion where it was purified on a 120 g silica column on a 0-100 ethyl acetate:cyclohexane gradient. The appropriate fractions were combined and concentrated to yield the title compound as an orange solid, 2 g.

LCMS (Method B); Rₜ=1 min, MH⁺=258.

### Intermediate 27

### 3-Fluoro-6-(1H-indol-4-yl)-1H-indazol-4-amine

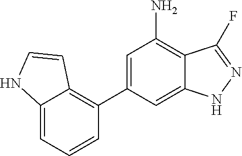

To a solution of 6-bromo-3-fluoro-1H-indazol-4-amine (0.25 g) in 1,4-dioxane (5 ml) was added 4-(4,4,5,5-tetramethyl-1,3,2-dioxaborolan-2-yl)-1H-indole (0.317 g), Pd(dppf)Cl₂ (0.04 g) followed by water (3.33 ml) and 2 M sodium carbonate (aq) (1.63 ml). The reaction vessel was sealed and heated in a microwave at 150° C. for 15 min. The sample was loaded onto a 5 g Silica cartridge and eluted with methanol (4 column volumes). The fractions were combined and evaporated to dryness to give a brown solid. A portion of this solid (0.2 g) was purified by MDAP (method A). The appropriate fraction was collected, evaporated to dryness, azeotroped with methanol (1×5 ml) then left under vacuum to give the title compound as a beige solid, 39 mg.

LCMS (method A); Rₜ=2.22 min, MH⁺=267.

The remainder (0.7 g) was dissolved in methanol (10 ml) and pre-adsorbed onto Fluorisil prior to purification on a 20 g silica SPE cartridge which was eluted with 0-100% ethyl acetate:cyclohexane over 40 min on the Flashmaster II. Appropriate fractions were combined and evaporated to give the title compound as a clear colourless foam, 210 mg.

LCMS (method A); Rₜ=2.21 min, MH⁺=267.

### Intermediate 28

### 6-Bromo-3-fluoro-1H-indazol-4-amine

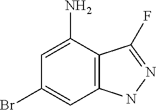

6-Bromo-3-fluoro-4-nitro-1H-indazole (1 g) was dissolved in methanol (20 ml) and acetic acid (5 ml). A small amount of solid remained that was removed by filtration. The resulting solution was pumped at 1.0 ml/min through an Omnifit column, which had been packed with iron granules (30 g). The column was heated to 100° C. via a Vapourtec R4 unit. Roughly 40 ml of solution was collected and evapourated to dryness to form a black residue. The residue was dissolved in dilute HCl—some of the solid did not dissolve and was filtered off. The solution was basified and extracted with ethyl acetate. Both layers were filtered through celite. The two layers were then separated and the aqueous phase extracted again with ethyl acetate. The combined organics were washed with brine, dried over MgSO₄, filtered and concentrated. The solid produced was then dissolved in DCM (10 ml) then loaded onto a 100 g silica column and purified on a 0-100% ethyl acetate:cyclohexane gradient. The relevant fractions were combined and concentrated to yield the title compound as a yellow solid, 250 mg.

LCMS (Method B) Rₜ=0.86 min, MH⁺=230.

### Intermediate 29

### N-{5-[4-Amino-1-(phenylsulfonyl)-1H-indazol-6-yl]-2-methyl-3-pyridinyl}methanesulfonamide

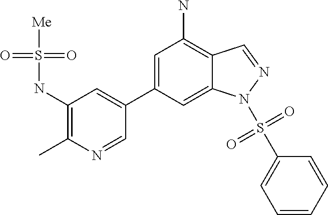

To an appropriate microwave reaction vial were introduced 1-(phenylsulfonyl)-6-(4,4,6,6-tetramethyl-1,3,2-dioxaborinan-2-yl)-1H-indazol-4-amine (300 ml), tripotassium phosphate (462 mg), Solvias catalyst (81 mg) and N-(5-bromo-2-methyl-3-pyridinyl)methanesulfonamide (212 mg). 1,4-Dioxane (3.5 ml) and water (0.7 ml) were added to the microwave reaction vial and the reaction was heated for a total of 10 min at 100° C. under microwave conditions. Solvias catalyst (40 mg) was added to the reaction mixture and the reaction mixture was heated again for 5 min at 100° C. The crude reaction mixture was washed through a preconditioned (with methanol) silica cartridge (2 g) using methanol. This crude was extracted between DCM and water and passed through a hydrophobic frit. The solution was dried down to obtain a solid that was purified using a Silica cartridge (5 g×2). The cartridge was preconditioned with DCM and the sample was loaded in DCM. Fractions were eluted with DCM and the final fraction with methanol. Appropriate fractions were combined and evaporated in vacuo then dried under high vacuum to yield the title compound, 241 mg.

LCMS (Method B); Rₜ=0.81 min, MH⁺=458.

### Intermediate 30

### 6-{6-Fluoro-1-[(4-nitrophenyl)sulfonyl]-1H-indol-4-yl}-1-(phenylsulfonyl)-1H-indazol-4-amine

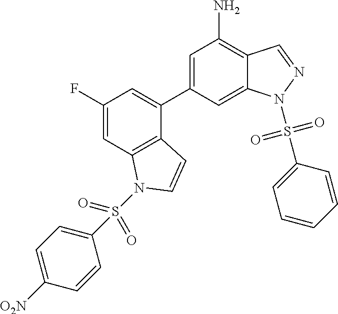

A mixture of 1-(phenylsulfonyl)-6-(trimethylstannanyl)-1H-indazol-4-amine (0.8 g), 4-bromo-6-fluoro-1-[(4-nitrophenyl)sulfonyl]-1H-indole (0.879 g) and Pd(PPh₃)₄ (0.212 g) in DMF (5 ml) was heated at 120° C. for 18 h. The solvent was removed in vacuo and the residue purified by silica cartridge (100 g) using a gradient of cyclohexane and ethyl acetate to give the title compound as an orange solid, 0.42 g.

LCMS (Method B); Rₜ=1.34 min, MH⁺=592.

### Intermediate 31

### 4-Bromo-6-fluoro-1-[(4-nitrophenyl)sulfonyl]-1H-indole

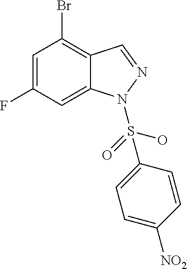

To a mixture of 4-bromo-6-fluoro-1H-indole (2 g) in DMF (5 ml) was added sodium hydride (60% in mineral oil) (0.448 g). The reaction was stirred at RT for 10 min. 4-Nitrobenzenesulfonyl chloride (2.278 g) was added and the reaction was stirred at RT for 1 h. The mixture was poured onto water (100 ml), and extracted with DCM (50 ml) which was separated by hydrophobic frit. Purification by silica (2×100 g) on Flashmaster II using a gradient of DCM and cyclohexane gave the title compound as a pale yellow solid, 1.54 g.

LCMS (Method B); Rₜ=1.39 min, MH⁺=400.

### Intermediate 32

### 3-Fluoro-6-{1-[(4-methylphenyl)sulfonyl]-1H-pyrazolo[3,4-b]pyridin-5-yl}-1-(phenylsulfonyl)-1H-indazol-4-amine

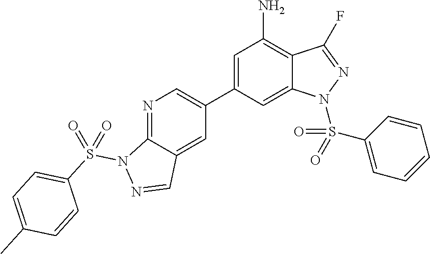

5-Bromo-1-[(4-methylphenyl)sulfonyl]-1H-pyrazolo[3,4-b]pyridine (605 mg) in DMF (4 ml) was added to a solution of 3-fluoro-1-(phenylsulfonyl)-6-(trimethylstannanyl)-1H-indazol-4-amine (650 mg) in DMF (6 ml), treated with Pd(PPh₃)₄ (165 mg) and then heated at 120° C. for 21 h. The solution was allowed to cool, filtered, and then evaporated. The residue was dissolved in chloroform, loaded onto a 100 g silica cartridge which was eluted with 0-100% ethyl acetate:cyclohexane over 60 min using the Flashmaster II. Appropriate peaks were combined and evaporated to give the title compound as a white solid, 0.577 g

LCMS (method A); Rₜ=3.28 min, MH⁺=563.

### Intermediate 33

### 5-Bromo-1-[(4-methylphenyl)sulfonyl]-1H-pyrazolo[3,4-b]pyridine

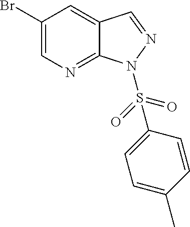

5-Bromo-1H-pyrazolo[3,4-b]pyridine (1.39 g) in DMF (6 ml) was cooled in an ice-bath under nitrogen and treated portion-wise with sodium hydride (0.308 g) (60% dispersion in oil) over a period of about 15 min. The reaction was left to stir in the ice-bath for 40 min, then treated with tosyl chloride (1.469 g) in DMF (2 ml). The reaction was left to stir in the ice-bath under nitrogen and the ice left to melt overnight. The reaction was stirred for a total of 20 h. The reaction was treated cautiously with water (6 ml) and stirred for 5 min. The reaction was poured onto water (60 ml), filtered and the residue treated with DCM (20 ml). The mixture was stirred, and then pushed through a frit into a cartridge with a hydrophobic frit. This procedure was repeated with further DCM (2×15 ml). The combined filtrates were evaporated to dryness to give the title compound as a brown solid, 1.802 g.

LCMS (method A); Rₜ=2.75 min, MH⁺=354.

### Intermediate 34

### 3-Fluoro-1-(phenylsulfonyl)-6-(trimethylstannanyl)-1H-indazol-4-amine

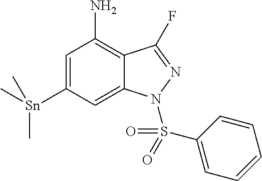

6-Bromo-3-fluoro-1-(phenylsulfonyl)-1H-indazol-4-amine (1.9 g), hexamethylditin (2.66 ml), triethylamine (1.431 ml) and Pd(PPh₃)₄ (0.593 g) were placed in toluene (30 ml). The mixture was split into 2 microwave vials and the mixtures heated at 110° C. for 1 h in the microwave. The mixtures were combined and poured onto a silica cartridge (50 g) that was eluted with cyclohexane followed by 1:1 cyclohexane: diethylether to give the title compound, 2.25 g.

LCMS (method A); Rₜ=3.46 min, MH⁺=456.

### Intermediate 35

### 6-Bromo-3-fluoro-1-(phenylsulfonyl)-1H-indazol-4-amine

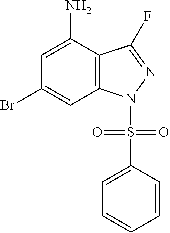

6-Bromo-3-fluoro-4-nitro-1-(phenylsulfonyI)-1H-indazole (5.9 g) was suspended in acetic acid (60 ml) and iron powder (4.12 g) was added. The suspension was heated to reflux for 2 h. The reaction was diluted with ethyl acetate (100 ml) and filtered through celite. The filter cake was washed well with ethyl acetate then the filtrate basified to around pH 8-9. The biphasic system was then stirred for 5 min. The layers were separated, the aq. washed with ethyl acetate and the combined organics washed with brine, dried over MgSO₄, filtered and concentrated in vacuum. The material was loaded onto a silica cartridge (330 g) and purified using a 0-100% ethyl acetate:cyclohexane gradient. The relevant fractions were combined and concentrated to yield the title compound as a yellow solid, 2.4 g.

LCMS (method B); Rₜ=1.17 min, MH⁺=370.

### Intermediate 36

### 6-Bromo-3-fluoro-4-nitro-1-(phenylsulfonyl)-1H-indazole

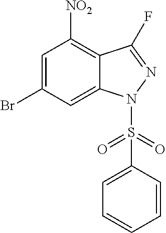

To a stirring suspension of sodium hydride (60% in mineral oil) (0.677 g) in THF (25 ml) at 0° C. was added a solution of 6-bromo-3-fluoro-4-nitro-1H-indazole (4 g) in THF (25 ml) dropwise. The reaction mixture was allowed to stir for half an hour then allowed to warm to RT before benzenesulfonyl chloride (2.17 ml) was added. After approximately two hours the reaction mixture was partitioned between ethyl acetate and water. The layers were separated and the aqueous was then extracted again with ethyl acetate. The combined organics were washed with brine, dried over MgSO₄, filtered and concentrated. The solid was triturated with methanol (50 ml) and filtered to yield the title compound as a yellow solid, 5.96 g.

LCMS (method B); Rₜ=1.27 min

### Intermediate 37

### N-(5-Bromo-3-pyridinyl)benzenesulfonamide

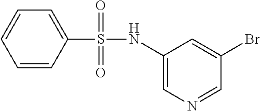

5-Bromo-3-pyridinamine (5 g) was dissolved in pyridine (150 ml) and the reaction was cooled to 0° C. in an ice-bath and stirred for 5 min. Benzenesulfonyl chloride (1.86 ml) was added dropwise and the reaction was stirred at 0° C. for 3 h. Further benzenesulfonyl chloride (1.86 ml) was added and the reaction continued stirring for 1 h. The reaction was neutralised with 2 M HCl (aq) and extracted with DCM (60 ml) then evaporated to dryness. The residue was dissolved in DCM and pre-adsorbed on fluorosil then purified by column chromatography on silica, eluting with 1-5% MeOH:DCM, to give the title compound, 8 g.

LCMS (method B); Rₜ=0.95 min, MH⁺=313.

### Intermediate 38

### N-(5-Bromo-2-methyl-3-pyridinyl)methanesulfonamide

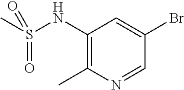

Methanesulfonyl chloride (97 μl) was added dropwise to 5-bromo-2-methyl-3-pyridinamine (200 mg) in pyridine (5 ml). The reaction was stirred at RT for 20 min before addition of 2M HCl (aq) (5 ml) and extraction into DCM. The DCM was passed through a hydrophobic frit then dried under a stream of nitrogen. The residue was dissolved in DCM and washed through an aminopropyl cartridge (preconditioned with DCM) with DCM to give the title compound, 77 mg.

LCMS (method B); Rₜ=0.77 min, MH⁺=267.

### Intermediate 39

### N-(5-Bromo-2-chloro-3-pyridinyl)benzenesulfonamide

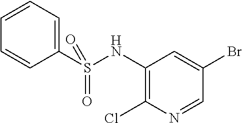

Benzenesulfonyl chloride (2.24 ml) was added dropwise over 5-10 min to a solution of 5-bromo-2-chloro-3-pyridinamine (2.5 g) and pyridine (1.5 ml) in DCM (25 ml). The reaction was stirred for 18 h at RT before evaporation to dryness in vacuo. The residue was split in two and purified by silica gel chromatography, eluting with 0-100% DCM in cyclohexane. Appropriate fractions were combined and evaporated to give the title compound, 1.81 g.

LCMS (Method B); Rₜ=1.09 min, MH⁻=347.

### Intermediate 40

### N-(5-Bromo-2-chloro-3-pyridinyl)methanesulfonamide

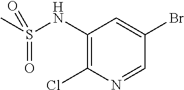

5-Bromo-2-chloro-3-pyridinamine (10 g) was dissolved in pyridine (75 ml) and methanesulfonyl chloride (7.5 ml) was added and stirred overnight. Further methanesulfonyl chloride (2.1 ml) was added and the reaction stirred at RT for 5 h. Further methanesulfonyl chloride (2.1 ml) was added and the mixture stired at RT overnight. The ph was adjusted to ˜6 by addition of 2M HCl (aq). The mixture was extracted with DCM (2×150 ml) and the combined organic layers were passed through a hydrophobic frit and the solvent removed in vacuo. The residue was suspended in methanol (200 ml) and 2M NaOH (50 ml) was added. The mixture was stirred for 1 h and then the solvent removed in vacuo. The residue was dissolved in water (250 ml) and washed with DCM (150 ml). The aqueous layer was acidified and the resulting precipitate collected by filtration. The solid was air dried overnight to give the title compound as an off white solid, 13.45 g.

LCMS (method B); Rₜ=0.81 min, MH⁻=285.

### Intermediate 41

### N-(5-Bromo-3-pyridinyl)methanesulfonamide

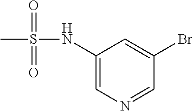

5-Bromo-3-pyridinamine (200 mg) was dissolved in pyridine (5 ml), cooled to 0° C. and stirred for 10 min. Methanesulfonyl chloride (0.05 ml) was added dropwise and the reaction continued stirring for 1 hr. 2M HCl (aq) (5 ml) was added and the reaction was extracted with DCM, passed through a hydrophobic frit and evaporated to dryness. The residue was dissolved in DCM and adsorbed onto fluorosil and purified by column chromatography on silica, eluting with 50% ether:cyclohexane then 0-10% MeOH:DCM to give the title compound, 96 mg.

LCMS (method B); Rₜ=0.68 min, MH⁺=251.

### Example 1

### 2-[(8aS)-Hexahydropyrrolo[1,2-a]pyrazin-2(1H)-ylmethyl]-N-[6-(1H-indol-4-yl)-1H-indazol-4-yl]-1,3-thiazole-4-carboxamide

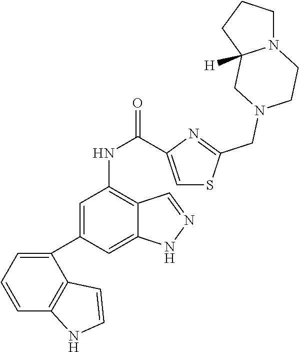

2-(Chloromethyl)-N-[6-(1H-indol-4-yl)-1-(phenylsulfonyl)-1H-indazol-4-yl]-1,3-thiazole-4-carboxamide (49 mg) and sodium iodide (13 mg) were dissolved in MeCN (1 ml) before (8aS)-octahydropyrrolo[1,2-a]pyrazine (23 mg) and DIPEA (0.031 ml) were added. The reaction mixture was heated at 70° C. for 1 h 30 min. The reaction mixture was cooled to RT then the solvent was removed under nitrogen. IPA (2 ml) and 2M NaOH (aq) (2 ml) were added and the reaction mixture was stirred for 30 h at RT. The reaction mixture was neutralised with 2M HCl (aq) until pH=7. The solvent was removed under nitrogen and the residue was dissolved in DMSO (2 ml), passed through a hydrophobic frit, then purified by MDAP (method A). The solvent was removed under nitrogen, and the residue was dissolved in water:dioxane (1:1) then freeze-dried to give the title compound, 7 mg.

LCMS (method B); Rₜ=0.58 min, MH⁺=498.

### Example 2

### 2-{[(3R,5S)-3,5-Dimethyl-4-(1-methylethyl)-1-piperazinyl]methyl}-N-[6-(1H-indol-4-yl)-1H-indazol-4-yl]-1,3-thiazole-4-carboxamide

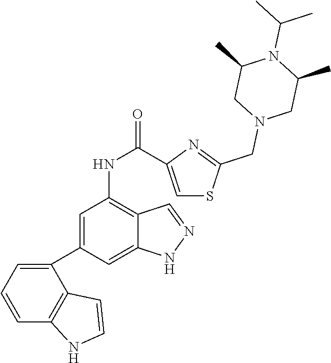

2-(Chloromethyl)-N-[6-(1H-indol-4-yl)-1-(phenylsulfonyl)-1H-indazol-4-yl]-1,3-thiazole-4-carboxamide (50 mg), (2R,6S)-2,6-dimethyl-1-(1-methylethyl)piperazine (which can be prepared as described in J. Med. Chem. 1999, 42, 1123-1144) (0.5 ml), potassium carbonate (38 mg) and MeCN (1 ml) were combined in a microwave vial then heated in the microwave for 15 min at 100° C. The solvent was removed under a flow of nitrogen. IPA (2 ml) and 2M NaOH (aq) (2 ml) were added and the reaction mixture was stirred for 72 h at RT before warming to 50° C. for 3 h 30 min. The reaction mixture was cooled then neutralised using 2M HCl (aq) until pH=7. The solvent was removed under a flow of nitrogen and the residue was dissolved in DMSO (2 ml), filtered and purified by MDAP (method A). The solvent was removed under a flow of nitrogen from the pure fraction to give the title compound as a white solid, 5 mg.

LCMS (method B); Rₜ=0.64 min, MH⁺=528.

### Example 3

### 2-{[(3R,5S)-3,5-Dimethyl-4-(1-methylethyl)-1-piperazinyl]methyl}-N-(6-{6-(methyloxy)-5-[(methylsulfonyl)amino]-3-pyridinyl}-1H-indazol-4-yl)-1,3-thiazole-4-carboxamide

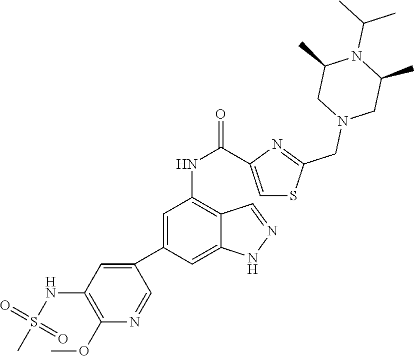

2-(Chloromethyl)-N-[6-{6-(methyloxy)-5-[(methylsulfonyl)amino]-3-pyridinyl}-1-(phenylsulfonyl)-1H-indazol-4-yl]-1,3-thiazole-4-carboxamide (40 mg), potassium carbonate (26 mg) and (2R,6S)-2,6-dimethyl-1-(1-methylethyl)piperazine (which can be prepared as described in J. Med. Chem. 1999, 42, 1123-1144) (10 mg) were added to MeCN (1 ml). The reaction mixture was heated in the microwave for 15 min at 110° C. The solvent was evaporated under a flow of nitrogen. IPA (2 ml) and 2M NaOH (aq) (2 ml) were then added and the reaction mixture was stirred for 30 h. The reaction was neutralised with 2M HCl (aq) to pH=7 and the solvent was evaporated under a flow of nitrogen. The residue was dissolved in DMSO (2 ml), filtered and purified by MDAP (method A). The pure fraction was evaporated under a flow of nitrogen, dissolved in dioxane:water (2 ml, 1:1, v/v) and freeze-dried to give the title compound as a white solid, 7 mg.

LCMS (method B); Rₜ=0.56 min, MH⁺=613.

### Example 4

### N-(6-{6-(Methyloxy)-5-[(methylsulfonyl)amino]-3-pyridinyl}-1H-indazol-4-yl)-1-(tetrahydro-2H-pyran-4-ylmethyl)-1H-pyrazole-3-carboxamide

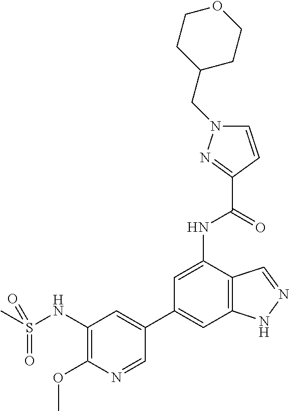

N-[2-(Methyloxy)-5-(4,4,5,5-tetramethyl-1,3,2-dioxaborolan-2-yl)-3-pyridinyl]methanesulfonamide (53 mg), sodium carbonate (46 mg) and Pd(dppf)Cl₂ (11 mg) were weighed to a microwave vial. N-[6-bromo-2-(tetrahydro-2H-pyran-2-yl)-2H-indazol-4-yl]-1-(tetrahydro-2H-pyran-4-ylmethyl)-1H-pyrazole-3-carboxamide (71 mg) was dissolved in 1,4-dioxane (1 ml) and added followed by water (1 ml). The reaction was heated in the microwave at 120° C. for 20 min. The reaction was passed through a 1 g silica cartridge, washing with methanol, then evaporated to dryness in the blow down. The residue was dissolved in DMSO:methanol (1 ml, 1:1. v/v) and purified by MDAP (method A). Pure fractions were combined, evaporated to dryness and the residue dissolved in dioxane:water (3 ml, 1:1, v/v) and freeze dried to give the title compound, 11 mg.

LCMS (method B); Rₜ=0.77 min, MH⁺=526.

### Example 5

### N-[6-(1H-Indol-4-yl)-1H-indazol-4-yl]-1-(tetrahydro-2H-pyran-4-ylmethyl)-1H-pyrazole-3-carboxamide

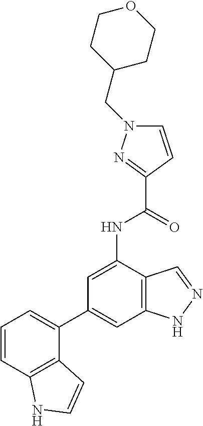

To a solution of 1-(tetrahydro-2H-pyran-4-ylmethyl)-1H-pyrazole-3-carboxylic acid (40 mg) in DMF (1 ml) was added HATU (69 mg) and the mixture allowed to stand for 10 min when 6-(1H-indol-4-yl)-2-(tetrahydro-2H-pyran-2-yl)-2H-indazol-4-amine (50 mg) and DIPEA (0.053 ml) were added and the mixture allowed to stand at RT for 18 h. The solvent was blown to dryness under a stream of nitrogen and purified by MDAP (method A). The crude product was dissolved in methanol (5 ml) and treated with 2 M HCl (aq) (1 ml) then blown to dryness under a stream of nitrogen to give the product as a pale brown solid that was purified by MDAP (method A) to give the title compound as a white solid, 16 mg.

LCMS (method A); Rₜ=2.36 min, MH⁺=441.

### Example 6

### N-[6-(1H-Indol-4-yl)-1H-indazol-4-yl]-1-(tetrahydro-2H-pyran-4-ylmethyl)-1H-pyrazole-5-carboxamide

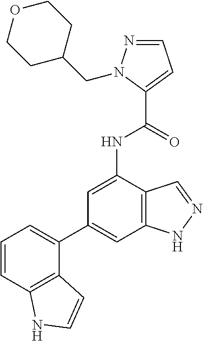

Prepared in a similar manner to Example 5, starting from 1-(tetrahydro-2H-pyran-4-ylmethyl)-1H-pyrazole-5-carboxylic acid, to give the title compound as a white solid, 16 mg.

LCMS (method A); Rₜ=2.3 min, MH⁺=441.

### Example 7

### 1-[(2-Chlorophenyl)methyl]-N-[6-(1H-indol-4-yl)-1H-indazol-4-yl]-1H-pyrazole-3-carboxamide

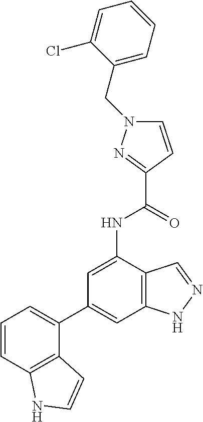

Prepared in a similar manner to Example 5, starting from 1-[(2-chlorophenyl)methyl]-1H-pyrazole-3-carboxylic acid to give the title compound as a white solid, 7 mg.

LCMS (method B); Rₜ=1.04 min, MH⁺=467.

### Example 8

### 1-[(4-Chlorophenyl)methyl]-N-[6-(1H-indol-4-yl)-1H-indazol-4-yl]-1H-pyrazole-3-carboxamide

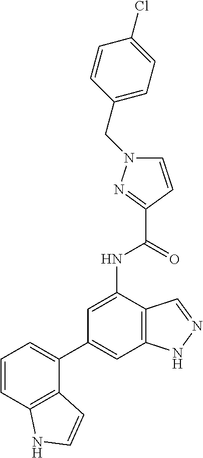

Prepared in a similar manner to Example 5, starting from 1-[(4-chlorophenyl)methyl]-1H-pyrazole-3-carboxylic acid to give the title compound as a white solid, 5 mg.

LCMS (method B); Rₜ=1.05 min, MH⁺=467.

### Example 9

### 6-[6-(1H-Indol-4-yl)-1H-indazol-4-yl]-6,7-dihydro-5H-pyrrolo[3,4-b]pyridin-5-one and

### Example 10

### 6-[6-(1H-Indol-4-yl)-1H-indazol-4-yl]-5,6-dihydro-7H-pyrrolo[3,4-b]pyridin-7-one

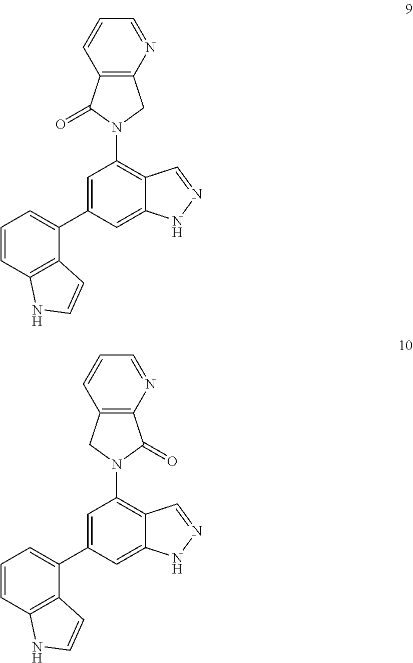

To a solution of dimethyl 2,3-pyridinedicarboxylate (1 g) in anhydrous toluene (25 ml), DIBAL (8.88 ml) was added slowly at −65° C. over 15-30 min. The reaction mixture was stirred at −65° C. for 30 min before quenching with 50% acetic acid (aq). The reaction mixture was extracted with toluene (2×50 ml) which was separated then evaporated to dryness. The residue was dissolved in ethyl acetate (10 ml), 6-(1H-indol-4-yl)-1H-indazol-4-amine (140 mg) and acetic acid (0.3 ml) were added and the reaction was stirred overnight at RT. Water (10 ml) was added then the reaction pH was adjusted to ca. 8-9 with triethylamine. The organic layer was separated and the aq. layer was extracted with of ethyl acetate (2×10 ml). The combined organic layer was evaporated and the residue was dissolved in methanol:DMSO (1:1, v/v, 0.5 ml) and purified by MDAP (method A). The residue was further purified by reverse-phase chromatography as follows;

Column: Supelco ABZ+ plus, 5 uM, 100×21.2 mm ID.

Mobile phase A: 0.1% formic acid (aq)

Mobile phase B: 0.1% formic acid:MeCN (5:95)

Flow rate: 20 ml/min

Gradient: 30-35% B over 25 mins.

Appropriate fractions were isolated and evaporated under a stream of nitrogen to give Example 9, 4 mg.

LCMS (method B); Rₜ=0.87 min, MH⁺=366.

Also isolated was Example 10, 3 mg.

LCMS (method B); Rₜ=0.85 min, MH⁺=366.

### Example 11

### N-[6-(1H-Indol-4-yl)-1H-indazol-4-yl]-2-(tetrahydro-2H-pyran-4-yl)acetamide

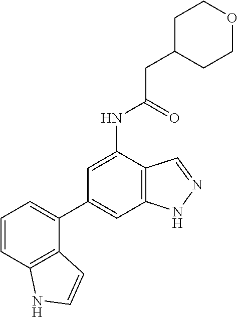

To a solution of tetrahydro-2H-pyran-4-ylacetic acid (35 mg) (available from Anichem) in DMF (1 ml) was added HATU (92 mg) and the mixture stirred at RT for 10 min when 6-(1H-indol-4-yl)-1H-indazol-4-amine (50 mg) and DIPEA (0.070 ml) were added and the mixture allowed to stand for 18 h. The mixture was blown to dryness under a stream of nitrogen and purified by MDAP (method A) to afford the title compound as a white solid, 16 mg.

LCMS (method A); Rₜ=2.1 min, MH⁺=375.

### Example 12

### N-[6-(1H-Indol-4-yl)-1H-indazol-4-yl]-2-(1-piperidinyl)acetamide

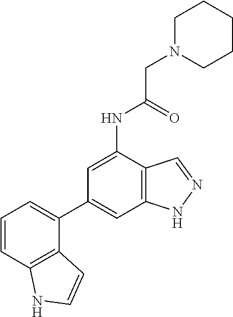

To a solution of 1-piperidinylacetic acid (35 mg) (available from Anichem) in DMF (1 ml) was added HATU (92 mg) and the mixture stirred at RT for 10 min when 6-(1H-indol-4-yl)-1H-indazol-4-amine (50 mg) and DIPEA (0.070 ml) were added and the mixture allowed to stand for 18 h. The mixture was blown to dryness under a stream of nitrogen and purified by MDAP (method A) to afford the title compound as a yellow solid, 15 mg. LCMS (method A); Rₜ=1.44 min, MH⁺=374.

### Example 13

### N-[6-(1H-Indol-4-yl)-1H-indazol-4-yl]-4-(4-morpholinyl)butanamide

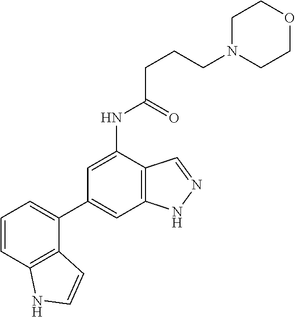

To a solution of 4-(4-morpholinyl)butanoic acid (42 mg) (available from ASDI) in DMF (1 ml) was added HATU (92 mg) and the mixture stirred for 10 min when 6-(1H-indol-4-yl)-1H-indazol-4-amine (50 mg) was added followed by DIPEA (0.07 ml) and the mixture stirred at RT for 18 h. The mixture was blown to dryness under a stream of nitrogen and purified by MDAP (method A) to afford the title compound as a white solid, 10 mg.

LCMS (method A); Rₜ=1.33 min, MH⁺=404.

### Example 14

### N-[6-(1H-Indol-4-yl)-1H-indazol-4-yl]-2-(4-morpholinyl)acetamide

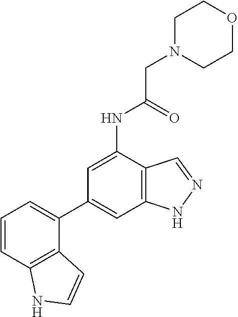

To a solution of 4-morpholinylacetic acid (48 mg) (available from ASDI) in DMF (1 ml) was added HATU (125 mg) and the mixture stirred for 10 min when 6-(1H-indol-4-yl)-1H-indazol-4-amine (68 mg) was added followed by DIPEA (0.096 ml) and the mixture allowed to stand for 18 h. The reaction was blown to dryness under a stream of nitrogen and purified by MDAP (method A) to afford the title compound as a white solid, 21 mg. LCMS (method A); Rₜ=1.32 min, MH⁺=376.

### Example 15

### 2-(4-Morpholinyl)-N-(6-{5-[(phenylsulfonyl)amino]-3-pyridinyl}-1H-indazol-4-yl)acetamide

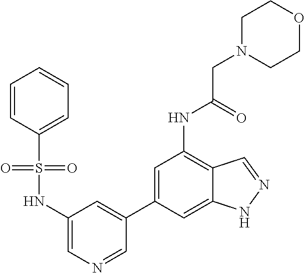

In an appropriate reaction vial was introduced N-{5-[4-amino-1-(phenylsulfonyl)-1H-indazol-6-yl]-3-pyridinyl}benzenesulfonamide (100 mg). To this was added a premixed solution of 4-morpholinylacetic acid (172 mg), DIPEA (0.207 ml and HATU (451 mg) in DMF (5 ml). After overnight stirring at RT, a further premixed solution of 4-morpholinylacetic acid (172 mg), DIPEA (0.207 ml) and HATU (451 mg) in DMF (2 ml) was added to the reaction. After stirring the reaction for 2 h at RT, a further premixed solution of 4-morpholinylacetic acid (172 mg), DIPEA (0.207 ml) and HATU (451 mg) in DMF (2 ml) was added and the reaction was left to stir overnight at RT. The reaction was purified using MDAP (method A) in 4 batches. The solid obtained from the first batch was dissolved in methanol (1 ml) and 2M NaOH (0.5 ml) was added. After 15 min at RT, 2M HCl (aq) was added until pH=7. The solution was dried down and the resulting solid was dissolved in methanol and passed through a preconditioned SCX cartridge (500 mg) with methanol. The cartridge was further eluted with 10% NH₃ in methanol keeping it separate from the methanol elute. The solutions were dried down to afford the title compound, 4 mg.

LCMS (method B); Rₜ=0.6 min, MH⁺=493.

The remaining 3 batches were also dried down, combined and treated in the same way as above to afford the title compound, 20 mg.

LCMS (Method B) Rₜ=0.6 min, MH⁺=493.

### Example 16

### N-(6-{6-Chloro-5-[(methylsulfonyl)amino]-3-pyridinyl}-1H-indazol-4-yl)-2-(4-morpholinyl)acetamide

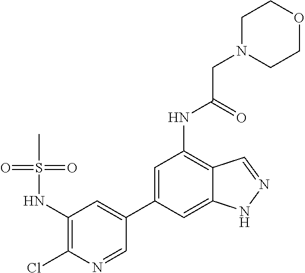

In an appropriate reaction vial was introduced N-{5-[4-amino-1-(phenylsulfonyl)-1H-indazol-6-yl]-2-chloro-3-pyridinyl}methanesulfonamide (50 mg). To this was added a premixed solution of 4-morpholinylacetic acid (30 mg), HATU (80 mg) and DIPEA (0.037 ml) in DMF (1 ml). After stirring at RT for 30 min, a further premixed solution of 4-morpholinylacetic acid (30 mg), DIPEA (0.037 ml) and HATU (80 mg) in DMF (0.5 ml) was added and the reaction mixture was left to stir at RT for 30 min. After this time the reaction was purified by MDAP (method A) and dried down to afford the protected product. The protected product was then dissolved in methanol (1 ml), treated with few drops of 2M sodium hydroxide (aq) and stirred for 10 min at RT. 2 M HCl (aq) was added until pH 7. The solution was dried down and the resulting solid was dissolved in methanol and washed through a preconditioned SCX cartridge (500 mg) with methanol. The product was eluted with 10% NH₃ in methanol and dried down to afford the title compound, 8 mg.

LCMS (method B); Rₜ=0.52 min, MH⁺=465.

### Example 17

### N-(6-{6-Methyl-5-[(methylsulfonyl)amino]-3-pyridinyl}-1H-indazol-4-yl)-2-(4-morpholinyl)acetamide

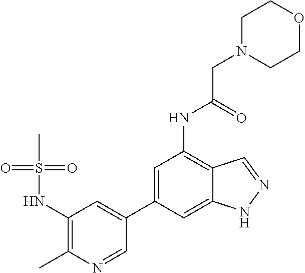

In an appropriate reaction vial was introduced N-{5-[4-amino-1-(phenylsulfonyl)-1H-indazol-6-yl]-2-methyl-3-pyridinyl}methanesulfonamide (50 mg). To this was added a premixed solution of 4-morpholinylacetic acid (30 mg), HATU (80 mg) and DIPEA (0.037 ml) in DMF (1 ml). After stirring at RT for 30 min, a further premixed solution of 4-morpholinylacetic acid (30 mg), DIPEA (0.037 ml) and HATU (80 mg) in DMF (0.5 ml) was added and the reaction was left to stir at RT for 30 min. After this time the reaction was purified by MDAP (method A) and dried down to afford the protected product. The protected product was then dissolved in methanol (1 ml), treated with few drops of 2M sodium hydroxide (aq) and stirred for 10 min at RT. 2M HCl (aq) was added until pH 7. The solution was dried down and the resulting solid was dissolved in methanol and washed through a preconditioned SCX cartridge (500 mg) with methanol. The product was eluted with 10% NH₃ in methanol and dried down to afford the title compound, 11 mg.

LCMS (method B); Rₜ=0.53 min, MH⁺=445.

### Example 18

### N-(6-{5-[(Methylsulfonyl)amino]-3-pyridinyl}-1H-indazol-4-yl)-2-(4-morpholinyl)acetamide

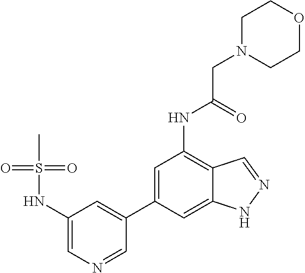

In an appropriate reaction vial was introduced N-{5-[4-amino-1-(phenylsulfonyl)-1H-indazol-6-yl]-3-pyridinyl}methanesulfonamide (50 mg). To this was added a premixed solution of 4-morpholinylacetic acid (30 mg), HATU (80 mg) and DIPEA (0.037 ml) in DMF (1 ml). After stirring at RT for 30 min, a further premixed solution of 4-morpholinylacetic acid (30 mg), DIPEA (0.037 ml) and HATU (80 mg) in DMF (0.5 ml) was added and the reaction was left to stir at RT for 30 min. After this time the reaction was purified by MDAP (method A) and dried down to afford the protected product. The protected product was then dissolved in methanol (1 ml), treated with few drops of 2M sodium hydroxide (aq) and stirred for 10 min at RT. 2M HCl (aq) was added until pH 7. The solution was dried down and the resulting solid was dissolved in methanol and washed through a preconditioned SCX cartridge (500 mg) with methanol. The product was eluted with 10% NH₃ in methanol and dried down to afford the title compound, 19 mg.

LCMS (Method B); Rₜ=0.43 min, MH⁺=431.

### Example 19

### N-(6-{6-Chloro-5-[(phenylsulfonyl)amino]-3-pyridinyl}-1H-indazol-4-yl)-2-(4-morpholinyl)acetamide

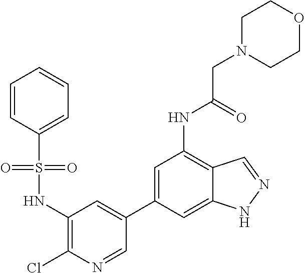

In an appropriate round bottom flask, N-{5-[4-amino-1-(phenylsulfonyl)-1H-indazol-6-yl]-2-chloro-3-pyridinyl}benzenesulfonamide (50 mg) was dissolved in DMF (1 ml). To this solution was added a premixed solution of 4-morpholinylacetic acid (27 mg), DIPEA (0.024 ml) and HATU (53 mg) in DMF (1 ml). After stirring the reaction for 30 min, a further premixed solution of 4-morpholinylacetic acid (27 mg), DIPEA (0.024 ml) and HATU (53 mg) was added in DMF (0.5 ml). After stirring the reaction at RT for 30 min, a further premixed solution of 4-morpholinylacetic acid (27 mg), DIPEA (0.024 ml) and HATU (53 mg) in DMF (0.5 ml) was added. After stirring the reaction for 30 min at RT, the reaction mixture was heated at 40° C. for 30 min. After this time, the reaction mixture was dried down, extracted between DCM and water and passed through a hydrophobic frit. The resulting organic layer was dried down, dissolved with a solution of DMSO:methanol (1:1, 1 ml) and purified by MDAP (method A) to afford the protected product. The protected product was then dissolved in methanol (2 ml), treated with a few drops of 2M sodium hydroxide (aq) and stirred for 5 min at RT. 2M HCl (aq) was added until pH 7. The solution was dried down and the resulting solid was dissolved in methanol and washed through a preconditioned SCX cartridge (500 mg) with methanol. The product was eluted with 10% NH₃ in methanol and dried down to afford the title compound, 4 mg.

LCMS (method B); Rₜ=0.69 min, MH⁺=527.

### Example 20

### N-[6-(6-Fluoro-1H-indol-4-yl)-1H-indazol-4-yl]-2-(4-morpholinyl)acetamide

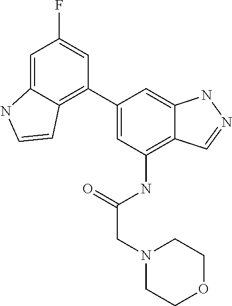

HATU (1.825 g) was dissolved in DMF (9.6 ml) and 1.6 ml of the resultant solution was dispensed to 4-morpholinylacetic acid (0.8 mmol) in DMF (1.6 ml). To this solution was added DIPEA (0.419 mL) and the mixture was left to stand for 5 minutes. 6-{6-Fluoro-1-[(4-nitrophenyl)sulfonyl]-1H-indol-4-yl}-1-(phenylsulfonyl)-1H-indazol-4-amine (0.6 mmol) was dissolved in DMF (1.2 ml) and 0.2 ml of the resultant solution was dispensed to an appropriate vial. To these vial was added the 4-morpholinylacetic acid:HATU solution, dispensed at 452 μl. The resulting solution was shaken for 5 min and left to stand at RT overnight. After this time, HATU (1.825 g) was dissolved in DMF (9.6 ml) and 1.6 ml of the resultant solution was dispensed to 4-morpholinylacetic acid (0.8 mmol) in DMF (1.6 ml). DIPEA (0.419 ml) was added to the 4-morpholinylacetic acid:HATU solution and the mixture was left to stand for 5 min. The 4-morpholinylacetic acid:HATU solution was added to the reaction mixture, dispensed at 452 μl. The solution was shaken for 5 min and placed in the oven at 40° C. for 1 h. DMF was removed in Genevac (not to dryness) and the compounds were dissolved in chloroform (300 μl). The solution was loaded onto an aminopropyl SPE cartridge (500 mg) that had been preconditioned with methanol followed by chloroform (2 ml each). The columns were eluted with 10% methanol in ethyl acetate (5 ml) and the fractions obtained were blown down under a stream of nitrogen. The samples were dissolved in DMSO (0.5 ml) and purified by MDAP (method B). The solvent was evaporated in vacuo using the Genevac to afford the required intermediate. This intermediate was dissolved in IPA (300 μl) and 2M sodium hydroxide (aq) (300 μl) was added. The solution was left for 32 h at RT. After this time the solution was neutralised with 2 M HCl (aq) and blown down under a stream of nitrogen. The sample was dissolved in DMSO (0.5 ml) and purified by MDAP (method C). The solvent was evaporated in vacuo using the Genevac. The residue was dissolved in 10% methanol in chloroform and added to the top of a 500 mg aminopropyl cartridge that had been pre-conditioned with methanol (1 column volume) followed by chloroform (1 column volume). The columns were eluted with 10% methanol in chloroform (1 column volume) and the fractions obtained were blown down under a stream of nitrogen to afford the title compound, 1.4 mg.

LCMS (method A) Rₜ=1.42 min, MH⁺=394.

### Example 21

### N-[3-Fluoro-6-(1H-pyrazolo[3,4-b]pyridin-5-yl)-1H-indazol-4-yl]-2-(4-morpholinyl)acetamide

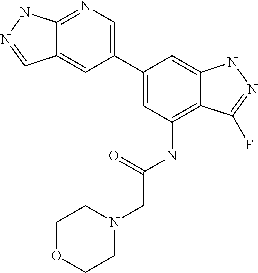

4-Morpholinylacetic acid was dissolved in THF (0.2 ml) and 1-chloro-N,N,2-trimethyl-1-propen-1-amine (15 μl) was added. The mixture was shaken and left to stand for 30 min. 3-Fluoro-6-{1-[(4-methylphenyl)sulfonyl]-1H-pyrazolo[3,4-b]pyridin-5-yl}-1-(phenylsulfonyl)-1H-indazol-4-amine (0.338 g) was suspended in THF (2.4 ml) and 0.4 ml of the resultant solution was added, followed by pyridine (16 μl). The reaction mixture was shaken and left to stand for 2 h. 4-Morpholinylacetic acid was dissolved in THF (0.2 ml) and 1-chloro-N,N,2-trimethyl-1-propen-1-amine (15 μl) was added. This mixture was shaken and left to stand for 30 min, then added to the reaction followed by pyridine (16 μl). The reaction was left to stand overnight. 4-Morpholinylacetic acid was dissolved in THF (0.2 ml) and 1-chloro-N,N,2-trimethyl-1-propen-1-amine (15 μl) was added. This mixture was shaken and left to stand for 30 min then added to the reaction, followed by pyridine (16 μl). The reaction was were stirred for more than 3 h. 4-Morpholinylacetic acid was dissolved in chloroform (0.2 ml) and 1-chloro-N,N,2-trimethyl-1-propen-1-amine (15 μl) was added. This mixture was shaken and left to stand for 30 min then added to the reaction, followed by pyridine (16 μl). The mixture was stirred for 30 min before blowing down under a stream of nitrogen. The sample was dissolved in DMSO (0.5 ml) and purified by MDAP (method C). The solvent was evaporated in vacuo using the Genevac to give the required intermediate. This was dissolved in IPA (300 μl) and 2M NaOH (aq) (300 μl) was added. The reaction was left over the weekend at RT. The solution was neutralised with 2M HCl (aq) and blown down under a stream of nitrogen. The sample was dissolved in DMSO (0.5 ml) and purified by MDAP (method C). The solvent was evaporated in vacuo using the Genevac to give the title compound, 4 mg.

LCMS (method A) Rₜ=1.29 min, MH⁺=396.

### Example 22

### N-[3-Fluoro-6-(1H-indol-4-yl)-1H-indazol-4-yl]-2-(4-morpholinyl)acetamide

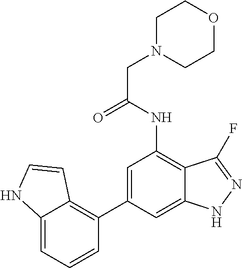

4-Morpholinylacetic acid HCl salt (0.043 g) in DMF (0.5 ml) was treated with HATU (0.089 g) and stirred for 20 min at RT. It was then treated with 3-fluoro-6-(1H-indol-4-yl)-1H-indazol-4-amine (0.052 g) in DMF (0.5 ml) followed by DIPEA (0.068 ml). The reaction mixture was stirred at RT for 66 h. The sample was purified directly by MDAP (method A). The major fraction was evaporated to dryness, azeotroped with methanol (1×5 ml) then left on the vacuum line for 2 h. The resulting gum was dissolved in methanol (1 ml) and loaded onto a 500 mg SCX cartridge which had been pre-rinsed with methanol. The column was washed with methanol (3 column volumes). Then 2M NH₃ in methanol (3 column volumes) was eluted and blown to dryness to give the title compound as a white solid, 25 mg.

LCMS (method A) Rₜ=1.55 min, MH⁺=394.

Biological Data

PI3K Alpha, Beta, Delta and Gamma Assays

Assay Principle

The assay readout exploits the specific and high affinity binding of PIP3 to an isolated pleckstrin homology (PH) domain in the generation of a signal. Briefly, the PIP3 product is detected by displacement of biotinylated PIP3 from an energy transfer complex consisting of Europium (Eu)-labelled anti-GST monoclonal antibody, a GST-tagged PH domain, biotin-PIP3 and Streptavidin-APC. Excitation of Eu leads to a transfer of energy to APC and a sensitized fluorescence emission at 665 nm. PIP3 formed by PI3kinase activity competes for the binding site on the PH domain, resulting in a loss of energy transfer and a decrease in signal.

Assay Protocol

Solid compounds are typically plated with 0.1 μl of 100% DMSO in all wells (except column 6 and 18) of a 384-well, v bottom, low volume Greiner plate. The compounds are serially diluted (4-fold in 100% DMSO) across the plate from column 1 to column 12 and column 13 to column 24 and leave column 6 and 18 containing only DMSO to yield 11 concentrations for each test compound.

The assays are run using specific PI3 kinase kits from Millipore (Cat #33-001)

The assay kit consist of the following:
4× PI3K reaction buffer (Contains 200 mM Hepes pH 7, 600 mM NaCl, 40 mM Mgcl₂, <1% Cholate (w/v), <1% Chaps (w/v), 0.05% Sodium Azide (w/v))
PIP2 (1 mM)
3× Biotin PIP3 (50 μM)
Detection Mix C (Contains 267 mM KF)
Detection Mix A (Contains 60 μg/ml streptavadin-APC)
Detection Mix B (Contains 36 μg/ml Europium-anti-GST (Anti-GST-K) and 90 μg/ml GST-GRP1-PH-Domain and 1 mM DTT)
Stop Solution (Contains 150 mM EDTA)

Manually add 3 μl of Reaction buffer (contains 1 mM DTT) to column 18 only for 100% inhibition control (no activity)

Manually add 3 μl of 2× Enzyme solution to all wells except column 18. Preincubate with compound for 15 minutes.

Manually add 3 μl of 2× Substrate solution to all wells.(column 6 represents 0% inhibition control)

Leave plate for 1 hr (cover from light) (In the case of Gamma only a 50 min incubation is required)

Manually add 3 μl Stop/Detection solution to all wells

Leave plate for 1 hour (cover from light)

The assay is read upon the BMG Rubystar and the ratio data is utilised to calculate 11 point curves.

NB The substrate solution (concentrations) differ with each isoform (see below)

Alpha

2× substrate solution containing 500 μM ATP, 16 μM PIP2 and 0.030 μM 3× biotin-PIP3.

Beta

2× substrate solution containing 800 μM ATP, 16 μM PIP2 and 0.030 μM 3× biotin-PIP3.

Delta

2× substrate solution containing 160 μM ATP, 10 μM PIP2 and 0.030 μM 3× biotin-PIP3.

Gamma

2× substrate solution containing 30 μM ATP, 16 μM PIP2 and 0.030 μM 3× biotin-PIP3.

Analysis Method

Data processed through the XC50 4-parameter logistic curve fit algorithm in Activity Base.

Normalise to % inhibition between the high and low controls (0% and 100% inhibition respectively)

Primary Module fit: Slope, Min and Max asymptotes varies

Secondary Module fits: (1) Fix Min asymptote, (2) Fix Max asymptote, (3) Fix Min and Max asymptotes

Curve Fit QC: pXC50 95% CL ratio>10

−20<Min asymptote<20

80<Max asymptote<120

The compounds of Examples 1 to 22 were tested in one or more of the PI3K Alpha, Beta, Delta and/or Gamma assays above or similar assays and were found to have a mean pIC₅₀ of 5 or greater.

What is claimed is:

1. A compound of formula (I) 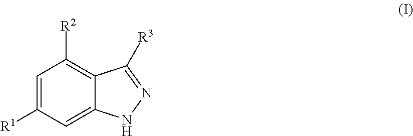
wherein
R¹ is 9- or 10-membered bicyclic heteroaryl wherein the 9- or 10-membered bicyclic heteroaryl contains from one to three heteroatoms independently selected from oxygen and nitrogen and is optionally substituted by C₁₋₆alkyl, C₃₋₆cycloalkyl, halo or —CN; or pyridinyl optionally substituted by one or two substituents independently selected from C₁₋₆alkyl, —OR⁴, halo, and —NHSO₂R⁵;
R² is —NHCOR⁶, 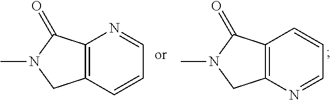
R³ is hydrogen or fluoro;
R⁴ and R⁷ are each independently hydrogen or C₁₋₆alkyl;
R⁵ is C₁₋₆alkyl or phenyl optionally substituted by one or two substituents independently selected from C₁₋₆alkyl, halo, —CF₃ and —OR⁷;
R⁶ is 5-membered heteroaryl wherein the 5-membered heteroaryl contains from one to three heteroatoms independently selected from oxygen, nitrogen and sulphur and is substituted by —CH₂NR⁸R⁹, —CH₂tetrahydropyran or —CH₂phenyl wherein the phenyl is substituted by halo; —(CH₂)ₙNR¹⁰R¹¹; or —CH₂tetrahydropyran;
R⁸ and R⁹, together with the nitrogen atom to which they are attached, are linked to form a 6-membered heterocyclyl or a 9-membered bicyclic heterocyclyl wherein the 6-membered heterocyclyl and the 9-membered bicyclic heterocyclyl each optionally containing a further nitrogen atom and the 6-membered heterocyclyl is substituted by three C₁₋₆alkyl substituents;
R¹⁰ and R¹¹, together with the nitrogen atom to which they are attached, are linked to form a 6-membered heterocyclyl optionally containing an oxygen atom; and
n is 1, 2 or 3;
or a salt thereof.

2. A compound according to claim 1 wherein R¹ is 9-membered bicyclic heteroaryl wherein the 9-membered bicyclic heteroaryl contains from one to three heteroatoms independently selected from oxygen and nitrogen and is optionally substituted by C₁₋₆alkyl, halo or —CN; or pyridinyl optionally substituted by one or two substituents independently selected from C₁₋₆alkyl, —OR⁴, halo, and —NHSO₂R⁵.

3. A compound according to claim 1 wherein R¹ is pyridinyl optionally substituted by one or two substituents independently selected from —OR⁴, halo, and —NHSO₂R⁵.

4. A compound according to claim 1 wherein R² is —NHCOR⁶.

5. A compound according to claim 1 wherein R³ is hydrogen.

6. A compound which is:
2-[(8aS)-hexahydropyrrolo[1,2-a]pyrazin-2(1H)-ylmethyl]-N-[6-(1H-indol-4-yl)-1H-indazol-4-yl]-1,3-thiazole-4-carboxamide;
2-{[(3R,5S)-3,5-dimethyl-4-(1-methylethyl)-1-piperazinyl]methyl}-N-[6-(1H-indol-4-yl)-1H-indazol-4-yl]-1,3-thiazole-4-carboxamide;
2-{[(3R,5S)-3,5-dimethyl-4-(1-methylethyl)-1-piperazinyl]methyl}-N-(6-{6-(methyloxy)-5-[(methylsulfonyl)amino]-3-pyridinyl}-1H-indazol-4-yl)-1,3-thiazole-4-carboxamide;
N-(6-{6-(methyloxy)-5-[(methylsulfonyl)amino]-3-pyridinyl}-1H-indazol-4-yl)-1-(tetrahydro-2H-pyran-4-ylmethyl)-1H-pyrazole-3-carboxamide;
N-[6-(1H-indol-4-yl)-1H-indazol-4-yl]-1-(tetrahydro-2H-pyran-4-ylmethyl)-1H-pyrazole-3-carboxamide;
N-[6-(1H-indol-4-yl)-1H-indazol-4-yl]-1-(tetrahydro-2H-pyran-4-ylmethyl)-1H-pyrazole-5-carboxamide;
1-[(2-chlorophenyl)methyl]-N-[6-(1H-indol-4-yl)-1H-indazol-4-yl]-1H-pyrazole-3-carboxamide;
1-[(4-chlorophenyl)methyl]-N-[6-(1H-indol-4-yl)-1H-indazol-4-yl]-1H-pyrazole-3-carboxamide;
6-[6-(1H-indol-4-yl)-1H-indazol-4-yl]-6,7-dihydro-5H-pyrrolo[3,4-b]pyridin-5-one;
6-[6-(1H-indol-4-yl)-1H-indazol-4-yl]-5,6-dihydro-7H-pyrrolo[3,4-b]pyridin-7-one;
N-[6-(1H-indol-4-yl)-1H-indazol-4-yl]-2-(tetrahydro-2H-pyran-4-yl)acetamide;
N-[6-(1H-indol-4-yl)-1H-indazol-4-yl]-2-(1-piperidinyl)acetamide;
N-[6-(1H-indol-4-yl)-1H-indazol-4-yl]-4-(4-morpholinyl)butanamide;
N-[6-(1H-indol-4-yl)-1H-indazol-4-yl]-2-(4-morpholinyl)acetamide;
2-(4-morpholinyl)-N-(6-{5-[(phenylsulfonyl)amino]-3-pyridinyl}-1H-indazol-4-yl)acetamide;
N-(6-{6-chloro-5-[(methylsulfonyl)amino]-3-pyridinyl}-1H-indazol-4-yl)-2-(4-morpholinyl)acetamide;
N-(6-{6-methyl-5-[(methylsulfonyl)amino]-3-pyridinyl}-1H-indazol-4-yl)-2-(4-morpholinyl)acetamide;
N-(6-{5-[(methylsulfonyl)amino]-3-pyridinyl}-1H-indazol-4-yl)-2-(4-morpholinyl)acetamide;
N-(6-{6-chloro-5-[(phenylsulfonyl)amino]-3-pyridinyl}-1H-indazol-4-yl)-2-(4-morpholinyl)acetamide;
N-[6-(6-fluoro-1H-indol-4-yl)-1H-indazol-4-yl]-2-(4-morpholinyl)acetamide;
N-[3-fluoro-6-(1H-pyrazolo[3,4-b]pyridin-5-yl)-1H-indazol-4-yl]-2-(4-morpholinyl)acetamide;
N-[3-fluoro-6-(1H-indol-4-yl)-1H-indazol-4-yl]-2-(4-morpholinyl)acetamide; or
a salt thereof.

7. A compound according to claim 1 the form of a pharmaceutically acceptable salt thereof.

8. A pharmaceutical composition comprising a compound as defined in claim 1, or a pharmaceutically acceptable salt thereof, and one or more pharmaceutically acceptable excipients.

9-11. (canceled)

12. A method of treating a disorder mediated by inappropriate PI3-kinase activity comprising administering a safe and effective amount of a compound as defined in claim 1, or a pharmaceutically acceptable salt thereof, to a patient in need thereof.

13. A method according to claim 12 wherein the disorder mediated by inappropriate PI3-kinase activity is a respiratory disease; an allergic disease; an autoimmune disease; an inflammatory disorder; a cardiovascular disease; a hematologic malignancy; cystic fibrosis; a neurodegenerative disease; pancreatitis; multiorgan failure; kidney disease; platelet aggregation; cancer; sperm motility; transplantation rejection; graft rejection; lung injury; or pain.

14. A method according to claim 12 wherein the disorder mediated by inappropriate PI3-kinase activity is asthma, chronic obstructive pulmonary disease (COPD), allergic rhinitis, atopic dermatitis, rheumatoid arthritis, multiple sclerosis, inflammatory bowel disease, thrombosis, atherosclerosis, hematologic malignancy, cystic fibrosis, neurodegenerative disease, pancreatitis, multiorgan failure, kidney disease, platelet aggregation, cancer, sperm motility, transplantation rejection, graft rejection, lung injury, pain associated with rheumatoid arthritis or osteoarthritis, back pain, general inflammatory pain, post hepatic neuralgia, diabetic neuropathy, inflammatory neuropathic pain (trama), trigeminal neuralgia or central pain.

15. A method according to claim 12 wherein the disorder mediated by inappropriate PI3-kinase activity is asthma.

16. A method according to claim 12 wherein the disorder mediated by inappropriate PI3-kinase activity is COPD.
